(19) United States
(12) Patent Application Publication
(10) Pub. No.: US 2010/0075308 A1
(43) Pub. Date: Mar. 25, 2010

(54) POLYMORPHISMS IN GENES AFFECTING CNS DISORDERS AND USES THEREOF

(75) Inventors: Wolfgang Sadee, Upper Arlington, OH (US); David Saffen, Columbus, OH (US); Julia Pinsonneault, Columbus, OH (US); Audrey Papp, Columbus, OH (US); Ying Zhang, Danbury, CT (US); Jeong-Eun Lim, Columbus, OH (US); Danxin Wang, Upper Arlington, OH (US)

Correspondence Address: CALFEE HALTER & GRISWOLD, LLP, 800 SUPERIOR AVENUE, SUITE 1400, CLEVELAND, OH, 44114, US

(73) Assignee: The Ohio State University Research Foundation, Columbus, OH (US)

(21) Appl. No.: 12/375,942

(22) PCT Filed: Aug. 1, 2007
(86) PCT No.: PCT/US07/75010

§ 371 Date: Nov. 13, 2009

Related U.S. Application Data
(60) Provisional application No. 60/821,077, filed on Aug. 1, 2006.
(60) Provisional application No. 60/926,932, filed on Apr. 30, 2007.

(51) Int. Cl. C12Q 1/68

(52) U.S. Cl. 435/6

(57) ABSTRACT

Diagnostic and prognostic methods, compositions, assays, and kits useful for predicting the phenotype of subjects who have, or are at risk of developing, a mental disorder. The methods also include predicting the prognostic outcome of a subject's mental disorder as well as the subject's responsiveness to drug treatments for the mental disorder. The methods and kits include determining the allelic status of polymorphisms in the MAOA, TPH2 and DRD2 genes.

### RELATED APPLICATIONS

This application claims priority to U.S. Provisional Application Ser. No. 60/926,932, filed Apr. 30, 2007, and U.S. Provisional Ser. No. 60/821,077, filed Aug. 1, 2006, the disclosures of both of which are incorporated herein by reference.

### GOVERNMENT SUPPORT

The invention was made with government support from the National Institutes of Health research grants DA022199 and DA021620; the National Institute of Drug Abuse grant R21 DA108744. The government may have certain rights in the invention.

### BACKGROUND

The neurotransmitters serotonin and dopamine play a critical role in numerous neuronal functions and their dysregulation is responsible for a large number of mental disorders.

Serotonin is mainly broken down by the enzyme monoamine oxidase A (MAOA). Because of the vital role that MAOA plays in serotonin inactivation, MAOA dysfunction (too much/too little MAOA activity) is thought to be responsible for a number of neurological disorders. For example, unusually high or low levels of MAOs in the body have been associated with depression, substance abuse, attention deficit disorder, and irregular sexual maturation, to name but a few. Monoamine oxidase inhibitors (MAOIs) are one of the major classes of drug prescribed for the treatment of depression.

Another enzyme that affects serotonin levels is Tryptophan hydroxylase (TPH). It catalyzes the rate-limiting step in the synthesis of Tryptophan hydroxylase 2 (TPH2), a recently discovered isoform of TPH that is specifically expressed in the brain, with particularly high expression in the serotonergic neurons of the raphe nuclei. The dorsal and media raphe nuclei are the major source of serotonin in the forebrain, including areas implicated in mood and anxiety disorders.

The dopaminergic system is involved in multiple neurophysiologic activities, such as cognition, learning and memory, and movement control; it is also the major substrate of reward and reinforcement of addictive drugs (e.g., alcohol, cocaine, and heroin). The dopamine D2 receptor (DRD2), distributed in both pre and post-synapses of neurons, is one of the primary targets for dopamine, and the dysfunction of DRD2 has been implicated in many neuropsychiatric disorders.

Disease susceptibility and therapeutic outcome of mental disorders vary with genetic variants in target genes (pharmacodynamics), such as those involved in neurotransmitter metabolism and signaling. Genetic variations in the genes that encode MAOA and TPH2 enzymes, as well as the gene encoding DRD2, have been studied intensely in an effort to uncover the genetic factors that predispose individuals to risk, determine prognosis, or affect response to therapy.

Yet it has been difficult to discover the genetic variants accounting for genetic risk, probably because multiple genes and gene combinations contribute. As a result, clinical linkage analysis and association studies have progressed rather slowly in discovering functional polymorphisms in candidate genes. While polymorphisms that change the amino acid sequence, such as non-synonymous SNPs, are readily studied in vitro for functional defects, recent surveys suggest that regulatory polymorphisms affecting gene transcription are more prevalent in the evolution of phenotypic traits. In addition, polymorphisms altering mRNA processing (maturation, splicing, etc) and turnover may be even more prevalent. These latter two types of polymorphisms are difficult to detect as they can appear anywhere in a given gene. Hence, there is a need for rapid and convenient methods of detecting functional polymorphisms that can act as biomarkers is assessing genetic risk for mental disorders.

In addition to the challenge of assessing an individual's risk of developing a mental disorder, another great clinical challenge is treating mental disorders and selecting the appropriate therapeutic agent. In usual clinical practice, this is largely trial and error, or drugs are chosen based on side effect profiles. There is also concern that many patients may never be tried on the agent that would best benefit them. There is therefore also a great need for a predictor that would aid clinicians in these difficult choices.

Functionally relevant polymorphisms in candidate genes have the potential of classifying patient populations (for example in depression) according to genetic factors, as a means for improving prediction of risk, prognosis, selection of drugs most likely to be active, and guiding drug development through preclinical and clinical trials (enhancing efficacy in a target population and reducing therapy failure or adverse effects).

### SUMMARY

The disclosure provides for a method for predicting a subject's risk for having or developing a mental disorder. The method includes detecting the allelic status of one or more polymorphisms in a nucleic acid sample of the subject, wherein the polymorphism is selected from the group: (i) monoamine oxidase A (MAOA)-associated SNPs rs6323, rs2205718, rs979606, rs979605, rs1801291, rs3027407 or combinations thereof; (ii) tryptophan hydroxylase 2 (TPH2)-associated SNP's rs2171363, rs4760815, rs7305115, rs6582078, rs9325202, or combinations thereof; (iii) DRD2-associated SNP's rs12364283; rs2283265; rs1076560 or combinations thereof; or (iv) a SNP in linkage disequilibrium with one or more SNPs listed in (i)-(iii). (see Table 1). In such a method, the allelic status of the polymorphism in the subject is predictive of the subject's risk for having or developing a mental disorder.

In one embodiment, the method further includes the step of correlating the allelic status of the polymorphism in the subject with the allelic status of the polymorphism in a reference population to predict the subject's risk for having or developing the mental disorder.

In another embodiment, the method further includes the step of correlating the allelic status of the polymorphism in the subject with the allelic status of the polymorphism in a reference population to predict whether the subject has a more or less severe phenotype of the mental disorder.

In another embodiment, the disclosure provides for a method of screening a subject for a prognostic biomarker of a mental disorder, comprising detecting the allelic status of one or more polymorphisms in a nucleic acid sample of the subject, wherein the polymorphism is selected from the group: (i) monoamine oxidase A (MAOA)-associated SNPs rs6323, rs2205718, rs979606, rs979605, rs1801291, rs3027407 or combinations thereof; (ii) tryptophan hydroxylase 2 (TPH2)-associated SNP's rs2171363, rs4760815, rs7305115, rs6582078, rs9325202, or combinations thereof; (iii) DRD2-associated SNP's rs12364283; rs2283265; rs1076560 or combinations thereof; or (iv) a SNP in linkage disequilibrium with one or more SNPs listed in (i)-(iii). In this method, the allelic status of the polymorphism in the subject is predictive of the prognostic outcome of the mental disorder.

In one embodiment, the method further includes the step of correlating the allelic status of the polymorphism in the subject with the allelic status of the polymorphism in a reference population to predict the prognostic outcome of the mental disorder in the subject.

In another embodiment, the method further includes the step of correlating the allelic status of the polymorphism in the subject with the allelic status of the polymorphism in a reference population to predict whether the subject has a more or less severe phenotype of the mental disorder.

In another embodiment, the method further includes the step of correlating the allelic status of the polymorphism in the subject with the allelic status of the polymorphism in a reference population to predict the subject's response to treatment.

In one embodiment, the mental disorder includes one or more of the following: substance abuse, attention deficit disorder (ADD), attention deficit hyperactivity disorder (ADHD), anxiety, depression, bipolar disorder, suicidal behavior, behavioral disorder, schizophrenia, Parkinson's disease or autism.

In one example, where the polymorphism is a TPH2-associated or MAOA-associated polymorphism, the mental disorder is one in which serotonin plays a role.

In another example, where the polymorphism is a DRD2-associated polymorphism, the mental disorder is one in which dopamine plays a role.

The SNPs identified herein can be used in combination with additional predictive tests including, but not limited to, additional SNPs, mutations, and clinical tests.

The disclosure also provides for a method for finding a functional polymorphism in a target gene implicated in a mental disorder, comprising: (i) providing a sample of a target tissue expressing the target gene; (ii) measuring the target gene's allelic mRNA expression imbalance (AEI) by quantitatively measuring the relative amounts of mRNA generated from each of two alleles in a transcribed region of the target gene and comparing the mRNA expression of one allele against the other allele to obtain an AEI ratio; and (iii) using the AEI ratio as a phenotype to scan the target gene for regions containing polymorphisms. Accordingly, a significant association between the AEI ratio and the polymorphism indicates that the polymorphism is a functional polymorphism that can serve as a biomarker for the mental disorder.

The present disclosure also relates to a kit comprising useful components for practicing the present method. A useful kit can contain oligonucleotide probes specific for MAOA, TPH2 or DRD2 alleles. The kit can also include instructions for correlating the assay results with the subject's risk for having or developing a mental disorder, the subject's prognostic outcome for the mental disorder, or the probability of success or failure of a particular drug treatment in the subject.

### BRIEF DESCRIPTION OF THE DRAWINGS

FIG. 1. Genomic structure of the MAOA gene, located on the X chromosome. The 15 exons are spread over 90.6 kilobases. Locations of genotyped SNPs are indicated by arrows. An asterisk (*) and a box marks the indicator SNPs in exon 8 and 14 used in allele-specific mRNA analysis.

FIG. 2. Pair-wise linkage disequilibrium (LD) data, allele frequency, chromosome position, location in introns and exons, and rs number for each genotyped SNP. Location of the polymorphisms in the MAOA locus is illustrated in FIG. 1. LD is expressed as D prime. Distances refer to base pairs (numbers in parentheses are in the 5′ direction). The SNP in exon 14 (rs1801291) was used as an indicator in allele-specific analysis. To accurately determine correct LD and allele frequencies, male genotype data were treated as homozygous (multiplied by two), while female genotype data were also doubled. D prime and allele frequencies were determined using HelixTree™ software (Lambert, C. (2004) HelixTree™ Genetics Analysis Software. 3.0.6 ed. Golden Helix, Inc., Bozeman, Mont.).

FIG. 3. MAOA Haplotypes with frequencies greater than 1%. The first column depicts haplotypes by genotype of each SNP. The second column depicts each haplotype by composition of major and minor alleles. Minor alleles are highlighted. “n” refers to the number of samples. There were 69 males and 36 females.

FIG. 4. Allele-specific measurements of mRNA, MAOA locus methylation ratios using Sma I, X inactivation and diplotypes for 17 female samples that are heterozygous for the marker SNP. A) Column I: Disease profile of individual sample. C=control, BP=bipolar, S=schizophrenia. Column II: Allelic DNA ratios, normalized to 1.0. Column III: Allele-specific mRNA ratios (mean=2.3, SD=1.0, n=3) of MAOA in 17 prefrontal cortex samples heterozygous for the marker SNP in exon 14, (except for Sample 451, which is heterozygous only for rs6323 in exon 8), normalized with DNA ratios. Average mRNA ratios and standard deviations are based on 3 independent measurements. Column IV: X-chromosome inactivation ratios measured as methylation at the androgen receptor gene locus using a polymorphic promoter repeat (ratio is high/low methylated allele. Phasing between the androgen receptor and MAOA on the X-chromosome is unknown. na: homozygous for the repeat polymorphism, nd: the reaction was not done or failed. Column V: Methylated MAOA DNA expressed as a ratio of 3-repeat over 4-repeat (results with Sma I). Column VI: Allelic C/T ratios adjusted for methylation by dividing C/T ratios by allele specific 3-repeat/4-repeat methylation ratios. Only samples that were heterozygous at both polymorphisms were adjusted. Columns VII and VIII: Haplotype assignments for the two alleles. Samples are heterozygous for the indicator SNP used here (in bold). “A” denotes the major allele and “B” the minor presented in order as shown in FIGS. 6 and 7 designate pVNTR repeats (no 5 repeats present). (*) homozygous for the pVNTR and heterozygous for marker SNP. B) Pair-wise Pearson correlations and p-values calculated for a three way comparison of MAOA expression ratios (A column III), X-inactivation ratios (A column IV) and methylated MAOA DNA ratios for the two alleles (3- and 4-repeat) (column V).

FIG. 5. MAOA allele expression imbalance of prefrontal cortex mRNA from 17 heterozygous female samples. These ratios are C allele/T allele (mean=2.0, SD=1.0, n=3). The plot is in log scale. A ratio of 1.0 represents equal amounts of each allele. These ratios were plotted on a log scale to equally represent the effect for ratios >1 and <1.

FIG. 6. Comparison of MAOA allelic expression imbalance obtained independently with two different marker SNPs (exon 8 and 14), in females heterozygous for both. Only one female (of 17) was heterozygous for the exon 8 SNP and homozygous for the exon 14 SNP, indicating a high degree of linkage.

FIG. 7. MAOA allelic expression imbalance ratios from 4 different brain regions in 4 individuals (ST255, ST380, ST381 and ST392). These ratios are C allele IT allele of the marker SNP in exon 14 (rs1801291) (n=3, except prefrontal cortex, with n=6).

FIG. 8. A) Allele-specific mRNA expression compared to X inactivation ratios. X-inactivation ratios were determined from the higher over the less highly methylated allele, since phasing between MAOA and the androgen receptor is unknown. B) Allele-specific mRNA expression compared to allele-specific methylation pattern. C/T ratios (exon 14 marker SNP) are correlated with the ratio of methylated 3-repeat/4-repeat allele. Pearson Correlation=0.83, Significance (2 tailed)=0.0008, R²=0.69. provided by Sma I locus. C). Allele-specific methylation ratios (Sma I locus) compared to X-inactivation ratios.

FIG. 9. Percent methylation of MAOA in females. A) The assay measured % methylation for each pVNTR allele specifically and overall. Methylation equivalents were determined by a standard curve with the equation % Meth=1.8088(Meth. peak/(Meth peak+Unmeth. peak))+0.0519. Mean and range/S.D. were determined for allele specific methylation from 2 or more methylation measurements. *: heterozygous for pVNTR and homozygous for marker SNP. B) Pairwise Pearson correlation and significance. 3-repeat/4-repeat ratios determined from these data were compared to C/T ratios in FIG. 4.

FIG. 10. SNP genotype linkage analysis to allelic expression of MAOA in females heterozygous for the marker SNP in exon 14. Each allele at each locus was tested whether it is associated with high or low expression. Allelic expression imbalance from sample ST451 was measured at the rs6323 locus.

FIG. 11. Box plot depicting overall expression of MAOA in male and female populations, sorted by pVNTR genotype in the top 2 panels and marker SNP genotype in the bottom 2 panels. The thick black line is the median, the top and bottom of boxes are the 3^{rd} and 1^{st} quartiles, respectively. Open circles (◯) signify outliers and asterisks (*) signify extreme outliers. Number of cases in each category for pVNTR: Females: 3/3 n=2; 3/4 n=23; 4/4 n=11. Males: 3 n=20; 4 n=46; 5 n=3. For marker SNP: Females: C/C n=16, C/T n=18, T/T n=2. Males: C n=52 T n=17.

FIG. 12. Transfected MAOA mRNA levels in CHO cells at various time points after transfection. RNA levels, expressed as arbitrary units, are derived from cycle threshold measurements after normalization to β-actin cycle thresholds.

FIG. 13. Primers for genotyping and SNaPshot™. All sequences are 5′-3′. rs979606 and rs979605 were genotyped using Applied Biosystems SNPlex™ genotyping system, according to manufacturers design. The pVNTR was genotyped with a fluorescent PCR primer, and the amplified product analyzed by capillary gel electrophoresis. All other SNPs were genotyped by allele-specific fluorescence PCR and melting curve analysis (Pinsonneault, J. and Sadee, W. (2003) AAPS PharmSci., 5, E29; incorporated herein by reference). Methylation assay is described in Methods section.

FIG. 14. mRNA sequence of the MAOA gene.

FIG. 15. Sequence of the MAOA 4-repeat pVNTR polymorphism. The 4 repeats which are highlighted in alternating yellow and blue.

FIG. 16—Haplotype structure of the human TPH2 gene and locations of key SNPs. The grey bar in the center of the figure represents the transcribed region of the TPH2 gene. Exons (1-11) are represented by vertical grey bars. The open bar below the transcribed region represents the segment of chromosome 12 (12q21) containing the TPH2 gene. The exact chromosomal location of this segment is indicated by the numbers at the beginning and end of the open bar. The vertical lines within the open bar denote the positions of the HapMap SNPs that were used for the determination of the haplotype structure of the TPH2 gene. The rs numbers for 11 HapMap SNPs examined in this study are listed below the open bar. The marker SNPs (rs7305115 and rs4290270) examined in this study are indicated in red type. The location of a rare missense mutation that reduces tryptophan hydroxylase activity (G1463A) is also indicated. The set of SNPs examined by Zill and coworkers in association studies of TPH2 and depression or suicide are annotated with the letters A though J. A SNP showing a statistically significant association with major depression (E: rs1386494) is marked with an asterisk (*). The triangular plot in the bottom half of the figure depicts estimated pairwise linkage disequilibrium (D′) values for HapMap SNPs. The plot was generated using the Haploview version 3.2 program with genotyping data from the CEU (Utah residents with ancestry from northern and western Europe) sample. Both the program and data set were downloaded from the International HapMap Project website. Red boxes indicate high estimated linkage disequilibrium (D′) between pairs of SNPs. Blue, pink and white boxes indicate lower estimated linkage disequilibrium (bright red: D′=1, LOD≧2; blue: D′=1, LOD<2; pink: D′<1, LOD≧2; white: D′<1, LOD<2).

FIG. 17. mRNA sequence of the TPH2 gene

FIG. 18. Comparison of genomic DNA and mRNA (cDNA) ratios assayed using the marker SNP rs7305115. Data are expressed as ratios of A:G alleles, corrected as described in Experimental Methods. The lightly shaded bars represent the average of three DNA ratio measurements using three independent preparations of pons genomic DNA. The darkly shaded bars represent the average of three mRNA ratio measurements using three independent cDNA preparations from a single preparation of pons total RNA. The error bars indicate (±) standard deviation (STDEV) for each set of measurements. Samples where the mRNA ratios are statistically different from 1.0 (P<0.001) using the GLM procedure in SAS are marked with an asterisk (*). Two genomic DNA samples (#1230 and #1609) that yielded AEI ratio significantly less than 1.0 are marked with arrowheads.

FIG. 19. Comparison of corrected genomic DNA and mRNA (cDNA) ratios assayed using the marker SNP rs4290270. Data are expressed as ratios of T:A alleles, as described in Experimental Methods. The lightly shaded bars represent the average of three DNA ratio measurements using three independent preparation of pons genomic DNA. The darkly shaded bars represent the average of three mRNA ratio measurements using three independent cDNA preparations from a single preparation of pons total RNA. The error bars indicate (±) standard deviation (STDEV) for each set of measurements Samples where the mRNA ratios are statistically different from 1.0 (P<0.001) using the GLM procedure in SAS are marked with an asterisk (*).

FIG. 20. Comparison of mRNA allelic expression ratios determined using the marker SNPs rs7305115 and rs4290270. The solid line represents the best fit for the data determined by linear regression, with the added requirement that the line pass through the origin, 0.0. (R=0.93; r²=0.86).

FIG. 21. A. D′ plot for the 22 SNPs listed in Table 3 (main text) based upon genotyping data from 36 Caucasian individuals in our collection. The plot was generated using Haploview (version 3.3; LD plot>Analysis>Solid Spine of LD, where the LD spine was extended if D′>0.7). Red boxes indicate high estimated linkage disequilibrium (D′) between pairs of SNPs. Blue, pink and white boxes indicate lower estimated linkage disequilibrium (bright red: D′=1, LOD≧2; blue: D′=1, LOD<2; pink: D′<1, LOD≧2; white: D′<1, LOD<2). Haplotype blocks demarcate segments of high linkage disequilibrium. Number within each square=D′×100. B. Estimated haplotypes and population frequencies for each haplotype block. Multiblock haplotypes are indicated by the lines between the blocks, with frequencies corresponding to the thickness of the lines. Observed frequencies of haplotypes within each block are listed in grey type. The numbers in black type are Hendrick multiallelic D′s, which estimate linkage disequilibrium between blocks by treating each block as an individual “allele.”

FIG. 22. Predicted diplotypes for individuals in samples. Diplotypes were predicted from genotyping data for the 48 individuals in our sample for the 22 SNPs listed in FIG. 23 using HelixTree™. Only one predicted diplotype is shown for cases where the estimation-maximum probability (EM-p) was 0.98 or greater. Accurate predictions could not be made for three SNPs (#19, 20, 21) in sample 1486: X=C/T; Y=A/T; Z=C/T. Alleles of SNPs for which heterozygosity is highly correlated with TPH2 AEI (Kappa coefficient>0.66) are listed in bold type.

FIG. 23. Information on the 22 SNP's used in the study. κ=2(ad−bc)/(p₁q₂+p₂q₁); where a=proportion of samples heterozygous & AEI(+); b=proportion of samples heterozygous & AEI (−); c=proportion of samples homozygous & AEI(+); d=proportion of samples homozygous & AEI(−); p₁=proportion of samples that are heterozygous for a give SNP (see FIG. 24); q₁=proportion of samples that are homozygous for a given SNP (see FIG. 24); p₂=proportion of samples that are AEI(+)=0.667 (18/27); q₂=proportion of samples that are AEI(−)=0.333 (9/27). Sample size=number of samples where AEI measurements were possible=number of samples heterozygous for marker SNP rs7305115 or rs4290270=27. κ>0.75: excellent agreement; 0.4 to 0.75=fair to good agreement; <0.4=poor agreement.

FIG. 24. Correlations between heterozygosity of individual TPH2 SNPs and allelic expression imbalance of TPH2 mRNA. Y-axis: Kappa-coefficients were calculated from data using SPSS. The values of Kappa-coefficients range from 1.0 for perfect correlation between heterozygosity and AEI (i.e., all samples heterozygous for the SNP show AEI and all homozygous samples show no AEI) and −1.0 for perfect anti-correlation (i.e., no samples heterozygous for the SNP show AEI and all homozygous samples show AEI). A SNP showing random correlations with AEI (i.e, 50% of heterozygous and homozygous samples show AEI) would have a Kappa value of 0.0. [(**): p<0.001; (*): p=0.003].

FIG. 25. TPH2 mRNA levels in pons measured using real-time PCR. The Y-axis plots the difference between cycle thresholds (C_{T}) determined for glyceraldehydes 3-phosphate dehydrogenase (GAPDH) and TPH2 mRNAs. Individuals where grouped according to their genotype for the marker SNP rs7305115: [G/G or G/A] (left) or [A/A] (right). Statistical significance was evaluated by the two-tailed t-test (p=0.0075).

FIG. 26. Comparison of TPH2 mRNA expression levels in different tissues. The Y-axis plots the difference between cycle thresholds (C_{T}) for GAPDH and TPH2 mRNAs. Results obtained from 27 pons samples, 5 non-pons brain regions (cerebellum and occipital, frontal, parietal and temporal cortexes) and 8 lymphoblast cell lines are shown. The pons sample set comprised individuals homozygous (A/A or G/G) for rs7305115 alleles. (One-way ANOVA; p<0.0001).

FIG. 27. Comparison of TPH2 mRNA stability for rs7305115 A- and G-alleles. A. Levels of TPH2 mRNA were quantified by real-time PCR at the indicated times (h) following transfection of CHO cells with an expression vector encoding human TPH2 (rs7305115 A-allele) at t=0. As indicated, highest levels of TPH2 A-allele mRNA were detected 24 h after transfection. Similar results were obtained following transfection of CHO cells with an expression vector encoding the TPH2 G-allele (data not shown). B. Allelic expression imbalance (AEI) assays for TPH2 A- and G-alleles were carried out using RNA isolated from CHO cells transfected with equal-molar amounts of expression vector encoding the TPH2 A- and TPH2 G-alleles. RNA was isolated at the indicated times following addition of 10 μg/ml actinomycin D (added 24 h after transfection). As indicted, AEI ratios did not change with time in either cells treated with actinomycin D (black bars) or not treated with actinomycin (grey bars). These data indicate that the rate of mRNA decay is the same for the TPH2 A- and G-alleles, both in the presence or absence of actinomycin D.

FIG. 28. Gene maps of DRD2, representing long and short splice variants. The locations of the 23 SNPs genotyped in this study are indicated by arrows.

FIG. 29. Comparison of allele specific expression of DRD2 mRNA using two indicator SNPs. Panel A. SNP21 versus SNP22 (Pearson r=0.9626, p<0.01); panel B: SNP20 versus SNP22 (Pearson r=0.931, p<0.0001).

FIG. 30. Allele-specific expression of DRD2 and SNP scanning Panel A. Allele-specific expression ratios in prefrontal cortex (54) and striatum (14). Samples are heterozygous for at least one of the marker SNPs, SNP20 (T/C; 47 subjects), SNP21 (C/T; 54) and SNP22 (C/G; 49). Data were normalized to DNA and are mean±SD (2 cDNA syntheses and 6 PCR reactions per sample). For subjects heterozygous for more than one marker SNP, ratios obtained with SNP21 were used, while SNP20 was used as marker in samples homozygous for SNP21. The bracket shows samples with significant AEI. Panel B. Association between SNPs and allelic expression imbalance (AEI) using HelixTree™ software. Adjusted P values were used to correct for multiple test effects.

FIG. 31. Genotyped SNPs of DRD2. Allele frequencies were calculated from the 105 samples of the Stanley Foundation (prefrontal cortex). Allele frequencies in the cohort of 100 subjects from the University of Bari are also provided where available.

FIG. 32. Panel A. Association between SNPs and allelic expression imbalance (AEI) using HelixTree™ software. Allelic ratios for 68 subjects are shown in FIG. 30, while the 23 SNPs are detailed in FIG. 28 and Table 7. Adjusted P values were used to correct for multiple test effects. Panel B. Association analysis between single SNPs and allelic expression imbalance (AEI) using all autopsied subjects (prefrontal cortex) but excluding individuals heterozygous for SNP2. The analysis was identical to that in FIG. 30B. No significant associations were observed.

FIG. 33. A. Predicted haplotype frequencies using 23 SNPs of DRD2 in Stanley samples (prefrontal cortex) (n=105). The SNPs are ordered from 1-23 as listed in Table 7 of the main text. The table includes only haplotypes with frequency more than 2%. B. Linkage disequilibrium analysis of 23 SNPs in DRD2 using HelixTree™ (n=105). The SNPs are ordered from 1-23 as listed in Table 7 of the main text. All SNPs were in Hardy-Weinberg equilibrium in the Stanley cohort, except for SNP23 (rs1800497) (p=0.03).

FIG. 34. Reporter gene assay testing SNP2. Panel A. Gene map showing amplified DRD2 promoter regions. Pro_LC/T1 and Pro_LC/T2 have 8 and 4 nucleotide deletion, respectively in the repeat region compared to the reference sequence (368 nts). Panel B. Luciferase activity of DRD2 promoters in HEK-293 and SH-SY5Y cells. Pro_L displayed greater promoter activity than Pro_S. The minor C allele of SNP2 conferred higher promoter activity than the T allele in both cell lines, regardless of repeat copy number (*p<0.05 and ***p<0.0001, one-way ANOVA, Bonferroni's multiple comparison test).

FIG. 35. Panel A. Allelic DRD2 expression ratios for total DRD2 mRNA, and for each splice variant (DRD2L and DRD2S) using marker SNP21 and SNP20. All 37 RNA samples were from the prefrontal cortex, including 30 heterozygous samples for SNP21 and 7 heterozygous for SNP20. Arrows indicate samples with significant differences of allelic ratios between DRD2L and S. Overall allelic ratios (T) including both splice variants are the same as shown in FIG. 30A. Panel B. SNP scanning of DRD2 using discrepant allelic mRNA expression between DRD2S and L. Allelic ratios were considered distinct between L and S if they differed by more than a factor of 1.25 (see Experimental for statistical analysis).

FIG. 36. Genotype effect on alternative DRD2 splicing. Panel A. SNP scanning of DRD2 using discrepant allelic mRNA expression between DRD2S and L. DRD2S and L allelic expression ratios in prefrontal cortex tissues are shown in FIG. 35. Panel B. Comparison of allelic mRNA ratios between DRD2L and DRD2S grouped by SNP19 genotype (SNP17 yields the same result). Data representing allelic ratios for DRD2L and DRD2S from the same subject are connected by solid lines. Panel C. Expression of DRD2S mRNA grouped by SNP19 genotypes (GG vs. GT+TT) in prefrontal cortex and striatum. Data are mean±SD, n=3, p<0.001 (prefrontal cortex, F=18.70, p<0.0001, n=40; in striatum, F=10.92, p=0.003, n=25 (one-way ANOVA)).

FIG. 37. Total mRNA expressions of DRD2 in two brain regions measured with RT-PCR. For each sample, the cycle thresholds (CT) for DRD2 and beta-actin were compared, and the differences in CT values for DRD2 and beta-actin were calculated. Lower cycle threshold numbers correspond to higher DRD2 mRNA expression. The expression of DRD2 mRNA is nearly two log orders higher in striatum compared to prefrontal cortex.

FIG. 38. Alternative splicing from DRD2 minigenes in HEK-293 cells. Minigene carrying 4 haplotypes of SNP17 (alleles: G (i5)/T (i5)) and SNP19 (G (i6)/T (i6)) were transfected into HEK cells. Data are mean±SD. The four haplotypes also carry two additional SNPs (shown in the minigene schematics) not associated with alternative splicing (FIG. 36A). *P<0.05, **P<0.01, ANOVA with Dunnett post test, compared to the main haplotype G (i5)/G (i6).

FIG. 39. GAA/GAAA repeat variants in the 5′ promoter region of DRD2. Primers were designed to amplify a DRD2 5′-upstream region including this repeat variant (−806˜−629 upstream of the transcription start site). 10 repeat variants of different lengths were detected in 105 prefrontal cortex DNA samples. Values are based on the migration distances during capillary electrophoresis on an ABI3730 (analyzed by GeneMapper software, Applied Biosystems Inc., CA). No significant association of these variants with AEI was discovered (adjusted p=0.31, HelixTree™).

FIG. 40. SNP19 genotype analysis of fMRI response during working memory task. Panel A: Results of ANOVA in SPM2 overlaid onto an average axial MRI at the level of the head of the caudate. The color bar indicates Z values of the difference in BOLD signal between the groups separated by GG and GT genotype. During the working memory task subjects with GT genotype had greater BOLD activity in bilateral head of the caudate compared with subjects with GG genotype. Panel B: Mean±0.95 standard error plots reflecting percent signal change from the cluster in left caudate head. Subjects with GT genotype had greater engagement of caudate head, compared with the homozygous GG subjects (one way ANOVA: F (1, 42)=18.950, p=0.00008).

FIG. 41. Demographics and working memory performance of subjects included in the fMRI analyses.

FIG. 42. Local maxima of brain regions crossing the statistical threshold in the SPM2 ANOVA comparing the effect of genotype at the two intronic SNPs (SNP17 and SNP19) on the fMRI data during working memory. Heterozygote subjects (GT) had greater activity than GG subjects. The Z value specifies the significance of observed differences; k=number of voxels within the cluster; x,y,z, Talairach coordinates of the center of mass.

FIG. 43. A. Oligonucleotides (primers) for genotyping using GC_Clamp PCR and SNaPshot assays. B. PCR conditions and oligonucleotide sequences for splice variant amplification and testing, promoter region constructs, repeat region detection, and minigenes.

FIG. 44. Standard curves for calculating relative mRNA expression of DRD2S and DRD2L using a fluorescently labeled forward primer. The assay conditions and primers are shown in FIG. 42. A series of mixtures of two cDNA plasmids (DRD2S and DRD2L) with varying ratios were used to obtain the standard curve, with a linear regression line showing a correlation coefficient of 0.998, p<0.0001.

FIG. 45. Alignment of DNA sequences flanking SNP2 from several species. The flanking sequence of SNP2 (rs12364283) (˜40 bp) is highly conserved between different species. In comparison with other species, the dominant allele ‘A’ of SNP2 is unique to humans. The flanking sequences contain putative sites for transcription factors, such as E47 (AT (C) CTGGC), ANF (GAATCTGGCAAA), NF-X3 (AGAATCTG), and HSF1 (long & short) (CACAGAAT) (TRANSFAC, version 8.3). Also, the minor C allele of SNP2 lacks binding sites for ANF and HSF1 but generates a new putative site for AREB6 (AGAACCTG, dissimilarity, 7.59%).

FIG. 46. mRNA sequence for the two variants of the DRD2 gene.

### DETAILED DESCRIPTION

The disclosure provides diagnostic and prognostic methods, compositions, assays, and kits useful for predicting the phenotype of subjects who have, or are at risk of developing, a mental disorder. The methods also include predicting the prognostic outcome of a subject's mental disorder as well as the subject's responsiveness to drug treatments for the mental disorder. The methods and kits include determining the allelic status of polymorphisms in the MAOA, TPH2 and DRD2 genes.

The disclosure also provides methods for identifying functional polymorphisms associated with one or more mental disorders using an allele-specific mRNA expression imbalance (AEI) assay combined with SNP scanning of a target gene locus with allelic mRNA ratios as a quantitative phenotype, together with in vitro molecular genetic analysis to identify the functional polymorphisms. Also provided are a number of functional single nucleotide polymorphisms (SNPs) in the MAOA, TPH2 and DRD2 genes.

AEI Assay

The question of how genetic processes interact to regulate gene expression can be addressed by measuring allelic expression imbalance (AEI). Measuring allelic mRNA expression compares one allele against the other in a relevant target tissue of the same individual. The assay quantitatively measures the relative amounts of mRNA generated from each of two alleles in physiologically relevant target tissues (e.g., specific brisan regions) from subjects heterozygous for a marker SNP in the transcribed region of the gene in question. AEI indicates the presence of cis-acting factors in gene regulation and/or mRNA processing. AEI results provide a quantitative measure of the allelic differences in each individual, one allele serving as the control for the other, while canceling out any trans-acting factors. The allelic expression ratios are then used as the phenotype to scan a gene locus for regions containing functional polymorphisms. If cis-acting polymorphisms contribute to the measured AEI ratios significant correlations should be detectable. For this analysis it is helpful to know the phasing of each SNP with the marker SNPs. As disclosed in the Examples, we conduct a single locus association test between SNP genotype and allelic expression phenotype. The AEI phenotype is represented either as present/absent; or absent/present low/present high, or as a continuous quantitative trait. Significant associations indicate that a SNP, or one closely linked, contributes to AEI, by affecting mRNA expression levels. These candidate polymorphisms, or haplotypes, are then cloned into expression vectors to determine the molecular mechanisms underlying the genetic changes where this is possible. A goal of this assay is to identify the polymorphisms that most closely account for any genetically based phenotypic differences between individuals.

Polymorphisms Linked to Function (AEI)

Using the above method, we were able to designate specific polymorphisms as biological biomarkers, used either alone of in combination with each other or with already established biomarkers. For each polymorphism in the candidate genes, we have established a link with allelic expression in human biopsy brain tissues as the phenotype. Obtained by scanning the entire gene in a number of individuals for polymorphisms that correlate with AEI, these polymorphisms are either directly responsible for altering mRNA expression, or they are in linkage disequilibrium or strong linkage disequilibrium with a functional SNP or SNPs. The listed polymorphisms are frequent (>5%), and have already shown statistically significant associations with clinical phenotypes. These polymorphisms therefore represent biallelic biomarkers associated with functional variants of key genes conveying susceptibility to CNS disorders and treatment outcome.

We disclose the use of AEI analysis to screen genes related to CNS disorders for functional polymorphisms. For three key candidate genes we have discovered frequent and substantial AEI across a number of individuals, indicating the presence of previously unknown and yet frequent functional polymorphisms. These genes are: MAOA, TPH2, and DRD2, encoding monoamine oxidase A, tryptophan hydroxylase 2, and dopamine receptor D2. These proteins are involved in biogenic amine metabolism and neuronal signaling, and appear to play a role in a spectrum of mental disorders. By scanning each gene in a number of individuals we have identified polymorphisms (SNPs) most closely related to the functional variation. Because these SNPs are linked to functional defects, and occur frequently in key candidate genes implicated in CNS disorders, they represent strong biomarkers for predicting individual risk and response to therapy. Because their functional significance is established, one can also analyze combinations of gene variants as risk factors, without greatly increasing the required statistical stringency for multiple comparisons.

In Example 1, we disclose the discovery of various polymorphisms strongly associated with AEI in the MAOA gene. Monoamine oxidase A (MAOA) is a candidate gene implicated in multiple CNS disorders, including, but not limited to, drug abuse, aggression, antisocial behavior, anxiety, attention deficit hyperactivity disorder, anorexia nervosa, bipolar disorder, and Alzheimer's disease. Monoamine oxidases catalyze the oxidation of biogenic amines and are the target of a class of antidepressant drugs. A repeat polymorphism in the promoter region of MAOA (pVNTR) has been extensively studied in vitro and in clinical association studies.

We show that the pVNTR and most other high frequency SNPs are linked with AEI. A block of 6 SNPs, including three intronic SNPs (rs2205718, rs979606 and rs979605), the marker SNP (rs1801291) and two other transcribed SNPs (rs6323 and rs3027407) are highly linked with each other and associated with AEI. On the other hand, the pVNTR is also in linkage disequilibrium with this 6-SNP haplotype block, but the linkage is less strong, and accordingly, the pVNTR is less tightly associated with the AEI ratios. This result indicates that the 6-SNP haplotype block located 3′ in the gene locus accounts for most of the observed AEI, and therefore, is a preferred choice as a biomarker.

Example 2 discloses the discovery of functional polymorphisms in the TPH2 gene. We have found five closely linked SNPs, rs2171363, rs4760815, rs7305115, rs6582078, and rs9325202, which show statistically significant correlations with TPH2 mRNA AEI, the minor allele being linked to enhanced TPH2 expression. rs7305115 appears to be functional, probably by enhancing correct splicing of the mRNA.

Example 3 discloses the discovery of functional polymorphisms in the DRD2 gene and their effect on dopaminergic neurotransmission. Several SNPs in DRD2 have shown to be associated with mental disorders such as schizophrenia and substance abuses, such as alcoholism, heroin abuse, and cigarette craving, but none of the previously identified SNPs are proven to be functional in vivo. We have discovered three functional polymorphisms rs12364283, rs2283265, and rs1076560, to be associated with AEI.

Example 4 discloses the role of the various MAOA, TPH2 and DRD2 functional polymorphisms in different mental disorders.

### DEFINITIONS

Unless defined otherwise, all technical and scientific terms used herein have the same meaning as commonly understood to one of ordinary skill in the art to which this disclosure belongs.

As used herein and in the appended claims, the singular forms “a,” “and,” and “the” include plural referents unless the context clearly dictates otherwise. Thus, for example, reference to “a polynucleotide” includes a plurality of such polynucleotides and reference to “the SNP” includes reference to one or more SNPs known to those skilled in the art, and so forth.

The term “allele” is used herein to refer to variants of a nucleotide sequence. Alleles are identified with respect to one or more polymorphic positions, with the rest of the gene sequence unspecified. For example, an allele may be defined by the nucleotide present at a single SNP; or by the nucleotides present at a plurality of SNPs, also termed haplotypes. A biallelic polymorphism has two forms. Diploid organisms may be homozygous or heterozygous for an allelic form.

For convenience, the allele present at the higher or highest frequency in the population will be referred to as the “main” or “wild-type” allele; less frequent allele(s) will be referred to as “minor” or “variant” allele(s).

Assessing the “allelic status” of a polymorphism refers to determining whether a subject is heterozygous (has one minor allele and one main allele), homozygous for the minor allele or homozygous for the main allele.

A “gene” refers to a segment of genomic DNA that contains the coding sequence for a protein, wherein the segment may include promoters, exons, introns, and other untranslated regions that control expression.

A “genotype” is an unphased 5′ to 3′ sequence of nucleotide pair(s) found at a set of one or more polymorphic sites in a locus on a pair of homologous chromosomes in a subject.

The term “genotyping” a sample or a subject for a polymorphism involves determining the specific allele or the specific nucleotide(s) carried by an individual at a biallelic marker.

The term “haplotype” refers to a combination of alleles present in an individual or a sample on a single chromosome. In the context of the present disclosure a haplotype refers to a combination of biallelic marker alleles found in a given individual and which may be associated with a phenotype.

“Haplotyping” is the process for determining one or more haplotypes in a subject and includes use of family pedigrees, molecular techniques and/or statistical inference.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A polymorphism may comprise a substitution, deletion or insertion of one or more nucleotides. A single nucleotide polymorphism (SNP) is a single base pair change. Typically a single nucleotide polymorphism is the replacement of one nucleotide by another nucleotide at the polymorphic site. Deletion of a single nucleotide or insertion of a single nucleotide, also give rise to single nucleotide polymorphisms. In the context of the present disclosure “single nucleotide polymorphism” refers to a single nucleotide substitution. Typically, between different genomes or between different individuals, the polymorphic site may be occupied by two different nucleotides.

The term “biallelic polymorphism,” “bialleleic marker,” or “biomarker” are used interchangeably and refer to a polymorphism having two alleles at a fairly high frequency in the population, sometimes a single nucleotide polymorphism. Typically the frequency of the less common allele of the biallelic polymorphism of the present disclosure has been validated to be greater than 1%, sometimes the frequency is greater than 10%, 20% (i.e. heterozygosity rate of at least 0.32), or 30% (i.e. heterozygosity rate of at least 0.42).

The term “mutation” refers to a difference in DNA sequence between or among different genomes or individuals that causes a functional change and which can have a frequency below 1%. Sequence variants describe any alteration in DNA sequence regardless of function or frequency.

“Linkage Disequilibrium” (“LD”) refers to alleles at different loci that are not associated at random, i.e., not associated in proportion to their frequencies. If the alleles are in positive linkage disequilibrium, then the alleles occur together more often than expected assuming statistical independence. Conversely, if the alleles are in negative linkage disequilibrium, then the alleles occur together less often than expected assuming statistical independence. As used herein, “strong linkage disequilibrium” is defined by D′ of >0.8.

As used interchangeably herein, the term “oligonucleotides”, and “polynucleotides” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide.

The term “purified” is used herein to describe a polynucleotide or polynucleotide vector of the disclosure which has been separated from other compounds including, but not limited to other nucleic acids, carbohydrates, lipids and proteins (such as the enzymes used in the synthesis of the polynucleotide), or the separation of covalently closed polynucleotides from linear polynucleotides.

The term “isolated” requires that the material be removed from its original environment (e.g., the natural environment if it is naturally occurring). For example, a naturally-occurring polynucleotide or polypeptide present in a living animal is not isolated, but the same polynucleotide or DNA or polypeptide, separated from some or all of the coexisting materials in the natural system, is isolated. Such polynucleotide could be part of a vector and/or such polynucleotide or polypeptide could be part of a composition, and still be isolated in that the vector or composition is not part of its natural environment.

The term “heterozygosity rate” is used herein to refer to the incidence of individuals in a population, which are heterozygous at a particular allele. In a biallelic system the heterozygosity rate is on average equal to 2 Pa(1-Pa), where Pa is the frequency of the least common allele. In order to be useful in genetic studies a genetic biomarker should have an adequate level of heterozygosity to allow a reasonable probability that a randomly selected person will be heterozygous.

The term “upstream” refers to a location which, is toward the 5′ end of the polynucleotide from a specific reference point. The term “downstream” refers to a location which is toward the 3′ end of the polynucleotide from a specific reference point.

The terms “base paired” and “Watson & Crick base paired” are used interchangeably herein to refer to nucleotides which can be hydrogen bonded to one another be virtue of their sequence identities in a manner like that found in double-helical DNA with thymine or uracil residues linked to adenine residues by two hydrogen bonds and cytosine and guanine residues linked by three hydrogen bonds (See Stryer, L., Biochemistry, 4th edition, 1995; incorporated herein by reference).

The terms “complementary” or “complement thereof” are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

The term “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase, or in a single nucleotide extension reaction for the measurement of AEI.

The term “probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified.

The primers and probes can be prepared by any suitable method, including, for example, cloning and restriction of appropriate sequences and direct chemical synthesis. The probes and primers can comprise nucleic acid analogs such as, for example peptide nucleic acids, locked nucleic acid (LNA) analogs, and morpholino analogs. The 3′ end of the probe can be functionalized with a capture or detectable label to assist in detection of a polymorphism.

Any of the oligonucleotides or nucleic acid of the disclosure can be labeled by incorporating a detectable label measurable by spectroscopic, photochemical, biochemical, immunochemical, or chemical means. For example, such labels can comprise radioactive substances (³²P, ³⁵S, ³H, ¹²⁵I)fluorescent dyes (5-bromodesoxyuridin, fluorescein, acetylaminofluorene, digoxigenin), biotin, nanoparticles, and the like. Such oligonucleotides are typically labeled at their 3′ and 5′ ends.

Probes can be used to detectably distinguish between target molecules differing in structure. Detection can be accomplished in a variety of different ways depending on the type of probe used and the type of target molecule. Thus, for example, detection may be based on discrimination of activity levels of the target molecule, but typically is based on detection of specific binding. Examples of such specific binding include antibody binding and nucleic acid probe hybridization. Thus, for example, probes can include enzyme substrates, antibodies and antibody fragments, and nucleic acid hybridization probes. Thus, in one embodiments, the detection of the presence or absence of the at least one variance involves contacting a target polymorphic site with a probe, typically an oligonucleotide probe, where the probe hybridizes with a form of the target nucleic acid containing a complementary base at the variance site as compared to hybridization to a form of the target nucleic acid having a non-complementary base at the variance site, where the hybridization is carried out under selective hybridization conditions. Such an oligonucleotide probe may span two or more variance sites. Unless otherwise specified, an oligonucleotide probe can include one or more nucleic acid analogs, labels or other substituents or moieties so long as the base-pairing function is retained.

A control population refers to a group of subjects or individuals who are predicted to be representative of the genetic variation found in the general population.

A “subject” comprises an individual (e.g., a mammalian subject or human) whose genotypes or haplotypes or response to treatment or disease state are to be determined.

A “nucleic acid sample” includes blood, serum, plasma, cerebrospinal fluid, urine, saliva, and tissue samples.

The term “phenotype” refers to any biochemically, anatomically, and clinically distinguishable, detectable or otherwise measurable property of an organism such as symptoms of, or susceptibility to a disease for example. Typically the term “phenotype” is used herein to refer to symptoms of, or susceptibility to a mental disorder; or to refer to an individual's response to an agent acting on a mental disorder; or to refer to symptoms of, or susceptibility to side effects to an agent acting on a mental disorder. A “less severe phenotype” is defined as a less severe form of a mental disorder, or a form of the mental disorder that is more responsive to treatment, displays less side effects with treatment, has better prognosis, is not recurrent, or has a combination of these characteristics. A “more severe phenotype” is defined as more severe form of a mental disorder, or a form of the mental disorder that is less responsive to treatment, displays more side effects with treatment, has worse prognosis, is recurrent, or has a combination of these characteristics. In general, the more severe phenotype is a disease state with profound consequences to the patient's life quality and requires more aggressive therapy.

A subject who is at risk for “having or developing a mental disorder” includes a subject with no clinical signs or symptoms of a mental disorder but with a strong family history of mental disorders, a subject who exhibits clinical signs or symptoms associated with a mental disorder, or a subject who has been clinically diagnosed as having a mental disorder.

The term “prognosis” as used herein refers to predicting the course or outcome of a condition in a subject. This does not refer to the ability to predict the course or outcome of a condition with 100% accuracy, or even that a given course or outcome is predictably more or less likely to occur based on the pattern of biomarkers. Instead, the skilled artisan will understand that the term “prognosis” refers to an increased probability that a certain course or outcome will occur.

A “diagnostic” biomarker is a biallelic polymorphism, the allelic status of which is indicative of whether or not a subject has, or is at risk for developing, a mental disorder.

A “prognostic” biomarker is a biallelic polymorphism, the allelic status of which is predictive of the severity or prognosis of a mental disorder.

When one or more prognostic biomarkers exhibit a certain pattern in samples obtained from a subject, the pattern may signal that the subject is at an increased probability for experiencing a future event in comparison to a similar subject exhibiting a different pattern. For example, a certain pattern of prognostic biomarkers can predict an increased predisposition to an adverse outcome, or the chance of a person responding or not responding to a certain drug.

In some embodiments, a “prognostic biomarker” can predict the presence of a “prognostic indicator.” For example, the presence of a minor allele of a SNP (prognostic biomarker) is indicative of a lower mRNA expression (prognostic indicator) in a target tissue.

The term “mental disorder” as used herein refers to any disorder in which an increase or decrease in available serotonin or dopamine contributes, at least in part, to a disease, disorder, or condition. Examples of such disorders include, but are not limited to: depression, anxiety, bipolar disorder, suicidal behavior, schizophrenia, autism, substance abuse (including alcoholism, tobacco abuse, symptoms caused by withdrawal or partial withdrawal from the use of tobacco or nicotine and drug addiction including cocaine abuse), attention-deficit disorder (ADD), attention-deficit hyperactivity disorder (ADHD), behavioral disorder, social phobia, disruptive behavior disorders, aggression, antisocial behavior, impulsive control disorders, borderline personality disorder, obsessive compulsive disorder, pathological gambling, novelty seeking, antisocial personality disorder, cognitive disorders, psychotic disorders, epilepsy, Tourette syndrome, mood disorders, panic disorder, eating disorders (including bulimia and anorexia nervosa), sleep disorders, migraine, obesity, premenstrual syndrome, menopause, fibromyalgia, neurodegenerative disorders (including Parkinsonism, dementia, dementia of ageing, senile dementia, prefrontal lobe dementia, Alzheimer's, and memory loss), and post-traumatic stress disorder (PTSD). All the above disorders have their usual meaning in the art, or are defined according to “The Merck Manual of Diagnosis and Therapy” Seventeenth Edition, 1999, Ed. Keryn A. G. Lane, pp. 1503-1598, incorporated herein by reference.

“Treatment” as used herein means the medical management of a subject, e.g., a human patient, with the intent to cure, ameliorate, stabilize, or prevent a disease, pathological condition, or disorder. This term includes active treatment, that is, treatment directed specifically toward the improvement or associated with the cure of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. “Treatment” also includes symptomatic treatment, that is, treatment directed toward constitutional symptoms of the associated disease, pathological condition, or disorder. “Treatment” also includes the act of not giving a subject a contra-indicated therapeutic.

A “serotonin enhancing drug” refers to therapeutic agents that increase the level of serotonin and can include, but is not limited to, a selective serotonin reuptake inhibitor (SSRI), a serotonin-norepinephrine reuptake inhibitor (SNRI), monoamine oxidase inhibitor (MOAI), a tricyclic antidepressant (TCA), an anxiolytic, a precursor or prodrug of serotonin, or an intermediate in serotonin biosynthesis.

The terms “correlating” as used herein refers to comparing the allelic status of a polymorphism in a subject to the allelic status of the polymorphism in a reference population. The reference population may be persons known to be free of a given condition, i.e. “normal individuals,” or may be persons known to suffer from, or to be at risk of developing, a given mental disorder, persons known to have a form of the mental disorder with better or worse outcome, or persons known to respond to or be resistant to a certain treatment. For example, a SNP pattern in a patient sample can be compared to a SNP pattern known to be associated with response to a certain depression medication. By correlating the sample's biomarker pattern with the reference pattern, the skilled artisan can predict whether the patient will respond to a certain medication, and prescribe accordingly.

Accordingly, the disclosure provides for a method for predicting a subject's risk for having or developing a mental disorder. The method includes detecting the allelic status of one or more polymorphisms in a nucleic acid sample of the subject, wherein the polymorphism is selected from the group: monoamine oxidase A (MAOA)-associated SNPs rs6323, rs2205718, rs979606, rs979605, rs1801291, rs3027407 or combinations thereof; tryptophan hydroxylase 2 (TPH2)-associated SNP's rs2171363, rs4760815, rs7305115, rs6582078, rs9325202, or combinations thereof; DRD2-associated SNP's rs12364283; rs2283265; rs1076560 or combinations thereof; or a SNP in linkage disequilibrium with one or more SNPs listed above. (see Table 1). In such a method, the allelic status of the polymorphism in the subject is predictive of the subject's risk for having or developing a mental disorder.

In one embodiment, the method further includes the step of correlating the allelic status of the polymorphism in the subject with the allelic status of the polymorphism in a reference population to predict the subject's risk for having or developing the mental disorder.

In another embodiment, the method further includes the step of correlating the allelic status of the polymorphism in the subject with the allelic status of the polymorphism in a reference population to predict whether the subject has a more or less severe phenotype of the mental disorder.

In another aspect, the disclosure provides for a method of screening a subject for a prognostic biomarker of a mental disorder, comprising detecting the allelic status of one or more polymorphisms in a nucleic acid sample of the subject, wherein the polymorphism is selected from the group: monoamine oxidase A (MAOA)-associated SNPs rs6323, rs2205718, rs979606, rs979605, rs1801291, rs3027407 or combinations thereof; tryptophan hydroxylase 2 (TPH2)-associated SNP's rs2171363, rs4760815, rs7305115, rs6582078, rs9325202, or combinations thereof; DRD2-associated SNP's rs12364283; rs2283265; rs1076560 or combinations thereof; or a SNP in linkage disequilibrium with one or more SNPs listed above. In this method, the allelic status of the polymorphism in the subject is predictive of the prognostic outcome of the mental disorder.

In one embodiment, the method further includes the step of correlating the allelic status of the polymorphism in the subject with the allelic status of the polymorphism in a reference population to predict the prognostic outcome of the mental disorder in the subject.

In another embodiment, the method further includes the step of correlating the allelic status of the polymorphism in the subject with the allelic status of the polymorphism in a reference population to predict whether the subject has a more or less severe phenotype of the mental disorder.

In another embodiment, the method further includes the step of correlating the allelic status of the polymorphism in the subject with the allelic status of the polymorphism in a reference population to predict the subject's response to treatment.

In one embodiment, the mental disorder includes one or more of the following: substance abuse, attention deficit disorder (ADD), attention deficit hyperactivity disorder (ADHD), anxiety, depression, bipolar disorder, suicidal behavior, behavioral disorder, schizophrenia, Parkinson's disease or autism.

For example, where the polymorphism is a TPH2-associated or MAOA-associated polymorphism, the mental disorder is one in which serotonin plays a role.

Similarly, where the polymorphism is a DRD2-associated polymorphism, the mental disorder is one in which dopamine plays a role.

The SNPs identified herein can be used in combination with additional predictive tests including, but not limited to, additional SNPs, mutations, and clinical tests.

The SNPs can be those provided in Table 1, below, and discussed in detail in the Examples. The SNPs can also be SNPs in positive linkage disequilibrium with any of the SNPs provided in Table 1.

TABLE 1
MAOA, TPH2 and DRD2 functional SNPs and their sequences

| | | | SNP | |
|---|---|---|---|---|
| Gene | rs Number | Sequence 5′ | main/minor | Sequence 3′ |
| MAOA | rs6323 | tccagcagagagaaaccagttaattcagcg | T/G | cttccaatgggagctgtcattaagtgcatg |
| | rs2205718 | gctctaaaaataacagaacctaagtgatga | C/A | taaaagaggccactgtgcaaatcatctcgt |
| | rs979606 | acaaaagagaaaacaaagctgaaatgctgc | A/G | agtcaataatatcgttgctttaacaaaaag |
| | rs979605 | ggattttgacaactatttctagaatttgca | C/T | tgaactctgcttttccttttaaatttggca |
| | rs1801291** | aaatggtctcgggaaggtgaccgagaaaga | C/T | atctgggtacaagaacctgaatcaaaggac |
| | rs3027407 | aaatttgactgttatttgttgagactatca | A/G | acagaaaagaaattagggctctaatttcct |
| TPH2 | rs2171363 | ttctttccttccccacctttggtgtttctg | C/T | cttgattgacatttctacctggcggttgga |
| | rs4760815 | cctcttcaaatcatgataatttatataaca | T/A | cacagaaaacaatatagagtgaaggagtta |
| | rs7305115 | ccggcatggctcagatcccctctacacccc | G/A | gaaccgtgagtacctacattaaagcccagg |
| | rs6582078 | gaacgtataacctgtgtgggagtctggaag | T/G | atgagcagaaatttcatttttgtacaaggc |
| | rs9325202 | tattatcattagtctctctgtatccctatc | G/A | tgcctttcttgagcagagagaccatctctt |
| DRD2 | rs12364283 | attagttaccaactgtcctcagtttgccag | A/G | ttctgtgtcagattcagaagtcacacacag |
| | rs1125394 | tacctggaagtcatgtgctttgtatgaaac | A/G | ccttggaatgctgataagtttaattctatt |
| | rs2075654 | tctttcttctagcacagtaattggcaataa | G/A | tggtcttatgtatctgggagaagataagcg |
| | rs2283265 | ctttttttgctgagtgaccttaggcaagtt | G/T | cttaccttctatgagcctgtttcctcatct |
| | rs1076560 | agccacccatctcactggcccctccctttc | C/A | ccctctgaagactcctgcaaacaccacagg |

**New SNP ID: rs1137070

The disclosure provides for various associations between disclosed SNPs and risk for a mental disorder, as follows:

MAOA SNPs

In one example, the MAOA polymorphism includes a 4 SNP haplotype comprising rs6323, rs2205718, rs979606, and rs979605.

In another example, MAOA polymorphism includes an MAOA-associated 6 SNP haplotype comprising rs6323, rs2205718, rs979606, rs979605, rs1801291, and rs3027407.

Accordingly, in one example, the presence of a minor allele of the 4 SNP or 6 SNP polymorphism mentioned above is predictive of lower levels of monoamine oxidase A in a target tissue (area of brain) associated with a mental disorder.

In another example, the presence of a minor allele of the 4 SNP or 6 SNP polymorphism mentioned above is predictive of an increased risk for aggression, substance abuse or antisocial behavior.

In another example, the polymorphism includes rs1801291 or a SNP in linkage disequilibrium with rs1801291. The method further includes detecting an MAOA-associated three-repeat or four-repeat pVNTR.

Accordingly, the presence of a minor allele of the rs1801291 polymorphism and the three-repeat or four-repeat pVNTR in a female subject is predictive of an increased risk for bipolar disorder.

In yet another example, the presence of a minor allele of the rs1801291 polymorphism and the three-repeat pVNTR in a female subject is predictive of an increased risk for suicidal behavior.

In another example, the presence of a minor allele of the rs1801291 polymorphism and the three-repeat or four-repeat pVNTR in a female subject is predictive of an increased resistance to serotonin enhancing drug therapy. In one aspect, the serotonin enhancing drug is a selective serotonin reuptake inhibitor (SSRI).

TPH2 SNPs

In one example, the TPH2 polymorphism includes a rs7305115 or a SNP in linkage disequilibrium with rs7305115. In one aspect, the presence of a minor allele of the polymorphism is predictive of higher levels of serotonin in a target tissue (area of brain) associated with a mental disorder. In another aspect, the presence of a minor allele of the polymorphism is predictive of a decreased risk for depression or suicidal behavior or both. In yet another aspect, the presence of a minor allele of the polymorphism is predictive of an increased resistance to serotonin enhancing drug therapy.

In another example, the TPH2 polymorphism includes a haplotype that includes rs7305115 in combination with one or more SNP's rs2171363, rs4760815, rs6582078, rs9325202. In one aspect, the presence of a minor allele of the haplotype is predictive of a reduced risk for depression or suicidal behavior or both.

In another example, the TPH2 polymorphism includes a 5 SNP haplotype TAAGA that includes minor alleles of rs2171363, rs4760815, rs7305115, rs6582078, and rs9325202. In one aspect, the presence of the 5 SNP haplotype TAAGA is predictive of high levels of TPH2 mRNA expression in the brain. In another aspect, the presence of the 5 SNP haplotype TAAGA is predictive of a reduced risk for depression or suicidal behavior or both. In yet another aspect, the presence of the 5 SNP haplotype TAAGA is predictive of resistance to serotonin enhancing drug therapy.

Combined MAOA and TPH2 SNPs

In one example, the biomarker includes at least one MAOA-associated polymorphism and one TPH2-associated polymorphism, selected from the following: (a) MAOA-associated SNPs rs6323, rs2205718, rs979606, rs979605, rs1801291, rs3027407 or a SNP in linkage disequilibrium with same; (b) TPH2-associated SNP's rs2171363, rs4760815, rs7305115, rs6582078, rs9325202; or a SNP in linkage disequilibrium with same.

In one aspect, the presence of a minor allele of such a combined polymorphism is predictive of a higher level of serotonin in a target tissue associated with the mental disorder.

In another aspect, the presence of a minor allele of the polymorphism is predictive of a decreased risk for depression or suicidal behavior or both.

DRD2 SNPs

In one example, the DRD2 polymorphism includes rs12364283 or a SNP in linkage disequilibrium with rs12364283. In one aspect, the presence of a minor allele of the polymorphism is predictive of a higher level of DRD2 mRNA expression in a target tissue associated a the mental disorder. In another aspect, the presence of a minor allele of the polymorphism is predictive of an increased risk for schizophrenia.

In another example, the DRD2 polymorphism includes rs2283265 or rs1076560, or a SNP in linkage disequilibrium with rs2283265 or rs1076560. In one aspect, the presence of a minor allele of the polymorphism is predictive of a higher level of DRD2L in a target tissue associated with a mental disorder. In another aspect, the presence of a minor allele of the polymorphism is predictive of enhanced dopaminergic neurotransmission in the subject. In yet another aspect, the presence of a minor allele of the polymorphism is predictive of an increased or decreased risk for a mental disorder involving memory loss.

In another example, the DRD2 polymorphism includes one or more minor alleles rs12364283, rs2283265 or rs1076560 or all three, and the presence of the polymorphism is predictive of an increased risk for schizophrenia.

In another example, the DRD2 polymorphism includes one or more of rs2283265, rs1076560 or both or a SNP in linkage disequilibrium with rs2283265, rs1076560 or both, and the presence of a minor allele of the polymorphism is predictive of an increased risk for depression. In one aspect, the SNP in linkage disequilibrium with rs2283265 is rs1125394, and the SNP in linkage disequilibrium with rs1076560 is rs2075654. In another aspect, the presence of a minor allele of the polymorphism is predictive of an increased or decreased responsiveness to psychotropic drug therapy

Genotyping Methods

In the methods of the present disclosure, the alleles present in a sample are identified by identifying the nucleotide present at one or more of the polymorphic sites. A number of methods are known in the art for identifying the nucleotide present at polymorphic sites. The particular method used to identify the genotype is not a critical aspect of the disclosure. Although considerations of performance, cost, and convenience will make particular methods more desirable than others, it will be clear that any method that can reliably identify the nucleotide present will provide the information needed to identify the genotype. Preferred genotyping methods involve DNA sequencing, allele-specific amplification, or probe-based detection of amplified nucleic acid.

MAOA, TPH2 or DRD2 alleles can be identified by DNA sequencing methods, such as the chain termination method (Sanger et al., 1977, Proc. Natl. Acad. Sci, 74:5463 5467, incorporated herein by reference), which are well known in the art. In one embodiment, a subsequence of the gene encompassing the polymorphic site is amplified and either cloned into a suitable plasmid and then sequenced, or sequenced directly. PCR-based sequencing is described in U.S. Pat. No. 5,075,216; Brow, in PCR Protocols, 1990, (Innis et al., eds., Academic Press, San Diego), chapter 24; and Gyllensten, in PCR Technology, 1989 (Erlich, ed., Stockton Press, New York), chapter 5; each incorporated herein by reference. Typically, sequencing is carried out using one of the automated DNA sequencers which are commercially available from, for example, PE Biosystems (Foster City, Calif.), Pharmacia (Piscataway, N.J.), Genomyx Corp. (Foster City, Calif.), LI-COR Biotech (Lincoln, Nebr.), GeneSys technologies (Sauk City, Wis.), and Visible Genetics, Inc. (Toronto, Canada).

The MAOA, TPH2 or DRD2 alleles can also be identified using amplification-based genotyping methods. Various nucleic acid amplification methods known in the art can be used in to detect nucleotide changes in a target nucleic acid. A preferred method is the polymerase chain reaction (PCR), which is now well known in the art, and described in U.S. Pat. Nos. 4,683,195; 4,683,202; and 4,965,188; each incorporated herein by reference. Examples of the numerous articles published describing methods and applications of PCR are found in PCR Applications, 1999, (Innis et al., eds., Academic Press, San Diego), PCR Strategies, 1995, (Innis et al., eds., Academic Press, San Diego); PCR Protocols, 1990, (Innis et al., eds., Academic Press, San Diego); and PCR Technology, 1989, (Erlich, ed., Stockton Press, New York); each incorporated herein by reference. Commercial vendors, such as PE Biosystems (Foster City, Calif.) market PCR reagents and publish PCR protocols.

Other suitable amplification methods include the ligase chain reaction (Wu and Wallace, 1988, Genomics 4:560 569); the strand displacement assay (Walker et al., 1992, Proc. Natl. Acad. Sci. USA 89:392 396, Walker et al. 1992, Nucleic Acids Res. 20:1691 1696, and U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al., 1989, Proc. Natl. Acad. Sci. USA, 86:1173 1177); and self-sustained sequence replication (3SR) (Guatelli et al., 1990, Proc. Natl. Acad. Sci. USA, 87:1874 1878 and WO 92/08800); each incorporated herein by reference. Alternatively, methods that amplify the probe to detectable levels can be used, such as Q.beta.-replicase amplification (Kramer et al., 1989, Nature, 339:401 402, and Lomeli et al., 1989, Clin. Chem., 35:1826 1831, both of which are incorporated herein by reference). A review of known amplification methods is provided in Abramson et al., 1993, Current Opinion in Biotechnology, 4:41 47, incorporated herein by reference.

MAOA, TPH2 or DRD2 alleles can also be identified using allele-specific amplification or primer extension methods, which are based on the inhibitory effect of a terminal primer mismatch on the ability of a DNA polymerase to extend the primer. To detect an allele sequence using an allele-specific amplification or extension-based method, a primer complementary to the MAOA, TPH2 or DRD2 genes is chosen such that the 3′ terminal nucleotide hybridizes at the polymorphic position. In the presence of the allele to be identified, the primer matches the target sequence at the 3′ terminus and primer is extended. In the presence of only the other allele, the primer has a 3′ mismatch relative to the target sequence and primer extension is either eliminated or significantly reduced. Allele-specific amplification- or extension-based methods are described in, for example, U.S. Pat. Nos. 5,137,806; 5,595,890; 5,639,611; and U.S. Pat. No. 4,851,331, each incorporated herein by reference.

Using allele-specific amplification-based genotyping, identification of the alleles requires only detection of the presence or absence of amplified target sequences. Methods for the detection of amplified target sequences are well known in the art. For example, gel electrophoresis (see Sambrook et al., 1989, supra.) and the probe hybridization assays described above have been used widely to detect the presence of nucleic acids.

Allele-specific amplification-based methods of genotyping can facilitate the identification of haplotypes, as described in the examples. Essentially, the allele-specific amplification is used to amplify a region encompassing multiple polymorphic sites from only one of the two alleles in a heterozygous sample. The SNP variants present within the amplified sequence are then identified, such as by probe hybridization or sequencing.

An alternative probe-less method, referred to herein as a kinetic-PCR method, in which the generation of amplified nucleic acid is detected by monitoring the increase in the total amount of double-stranded DNA in the reaction mixture, is described in Higuchi et al., 1992, Bio/Technology, 10:413 417; Higuchi et al., 1993, Bio/Technology, 11:1026 1030; Higuchi and Watson, in PCR Applications, supra, Chapter 16; U.S. Pat. Nos. 5,994,056 and 6,171,785; and European Patent Publication Nos. 487,218 and 512,334, each incorporated herein by reference. The detection of double-stranded target DNA relies on the increased fluorescence that DNA-binding dyes, such as ethidium bromide, exhibit when bound to double-stranded DNA. The increase of double-stranded DNA resulting from the synthesis of target sequences results in an increase in the amount of dye bound to double-stranded DNA and a concomitant detectable increase in fluorescence. For genotyping using the kinetic-PCR methods, amplification reactions are carried out using a pair of primers specific for one of the alleles, such that each amplification can indicate the presence of a particular allele. By carrying out two amplifications, one using primers specific for the wild-type allele and one using primers specific for the mutant allele, the genotype of the sample with respect to that SNP can be determined. Similarly, by carrying out four amplifications, each with one of the possible pairs possible using allele specific primers for both the upstream and downstream primers, the genotype of the sample with respect to two SNPs can be determined. This gives haplotype information for a pair of SNPs.

Alleles can be also identified using probe-based methods, which rely on the difference in stability of hybridization duplexes formed between a probe and its corresponding target sequence comprising an allele of interest. Under sufficiently stringent hybridization conditions, stable duplexes are formed only between a probe and its target allele sequence and not other allele sequences. The presence of stable hybridization duplexes can be detected by any of a number of well known methods. In general, amplify a nucleic acid encompassing a polymorphic site of interest prior to hybridization in order to facilitate detection. However, this is not necessary if sufficient nucleic acid can be obtained without amplification.

A probe suitable for use in the probe-based methods of the present disclosure, which contains a hybridizing region either substantially complementary or exactly complementary to a target region that encompasses the polymorphic site, and exactly complementary to one of the two allele sequences at the polymorphic site, can be selected using the guidance provided herein and well known in the art. Similarly, suitable hybridization conditions, which depend on the exact size and sequence of the probe, can be selected empirically using the guidance provided herein and well known in the art. The use of oligonucleotide probes to detect nucleotide variations including single base pair differences in sequence is described in, for example, Conner et al., 1983, Proc. Natl. Acad. Sci. USA, 80:278 282, and U.S. Pat. Nos. 5,468,613 and 5,604,099, each incorporated herein by reference.

In preferred embodiments of the probe-based methods for determining the MAOA, TPH2 or DRD2 genotypes, multiple nucleic acid sequences from the MAOA, TPH2 or DRD2 genes which encompass the polymorphic sites are amplified and hybridized to a set of probes under sufficiently stringent hybridization conditions. The alleles present are inferred from the pattern of binding of the probes to the amplified target sequences. In this embodiment, amplification is carried out in order to provide sufficient nucleic acid for analysis by probe hybridization. Thus, primers are designed such that regions of the MAOA, TPH2 or DRD2 genes encompassing the polymorphic sites are amplified regardless of the allele present in the sample. Allele-independent amplification is achieved using primers which hybridize to conserved regions of the genes. The genes contain many invariant or monomorphic regions and suitable allele-independent primers can be selected routinely. One of skill will recognize that, typically, experimental optimization of an amplification system is helpful.

Suitable assay formats for detecting hybrids formed between probes and target nucleic acid sequences in a sample are known in the art and include the immobilized target (dot-blot) format and immobilized probe (reverse dot-blot or line-blot) assay formats. Dot blot and reverse dot blot assay formats are described in U.S. Pat. Nos. 5,310,893; 5,451,512; 5,468,613; and 5,604,099; each incorporated herein by reference.

In a dot-blot format, amplified target DNA is immobilized on a solid support, such as a nylon membrane. The membrane-target complex is incubated with labeled probe under suitable hybridization conditions, unhybridized probe is removed by washing under suitably stringent conditions, and the membrane is monitored for the presence of bound probe. A preferred dot-blot detection assay is described in the examples.

In the reverse dot-blot (or line-blot) format, the probes are immobilized on a solid support, such as a nylon membrane or a microtiter plate. The target DNA is labeled, typically during amplification by the incorporation of labeled primers. One or both of the primers can be labeled. The membrane-probe complex is incubated with the labeled amplified target DNA under suitable hybridization conditions, unhybridized target DNA is removed by washing under suitably stringent conditions, and the membrane is monitored for the presence of bound target DNA. A preferred reverse line-blot detection assay is described in the examples.

Probe-based genotyping can be carried out using a “TaqMan” or “5′-nuclease assay,” as described in U.S. Pat. Nos. 5,210,015; 5,487,972; and 5,804,375; and Holland et al., 1988, Proc. Natl. Acad. Sci. USA, 88:7276 7280, each incorporated herein by reference. In the TaqMan assay, labeled detection probes that hybridize within the amplified region are added during the amplification reaction mixture. The probes are modified so as to prevent the probes from acting as primers for DNA synthesis. The amplification is carried out using a DNA polymerase that possesses 5′ to 3′ exonuclease activity, e.g., Tth DNA polymerase. During each synthesis step of the amplification, any probe which hybridizes to the target nucleic acid downstream from the primer being extended is degraded by the 5′ to 3′ exonuclease activity of the DNA polymerase. Thus, the synthesis of a new target strand also results in the degradation of a probe, and the accumulation of degradation product provides a measure of the synthesis of target sequences.

Any method suitable for detecting degradation product can be used in the TaqMan assay. In a preferred method, the detection probes are labeled with two fluorescent dyes, one of which is capable of quenching the fluorescence of the other dye. The dyes are attached to the probe, sometimes one attached to the 5′ terminus and the other is attached to an internal site, such that quenching occurs when the probe is in an unhybridized state and such that cleavage of the probe by the 5′ to 3′ exonuclease activity of the DNA polymerase occurs in between the two dyes. Amplification results in cleavage of the probe between the dyes with a concomitant elimination of quenching and an increase in the fluorescence observable from the initially quenched dye. The accumulation of degradation product is monitored by measuring the increase in reaction fluorescence. U.S. Pat. Nos. 5,491,063 and 5,571,673, both incorporated herein by reference, describe alternative methods for detecting the degradation of probe which occurs concomitant with amplification.

The TaqMan assay can be used with allele-specific amplification primers such that the probe is used only to detect the presence of amplified product. Such an assay is carried out as described for the kinetic-PCR-based methods described above. Alternatively, the TaqMan assay can be used with a target-specific probe.

Examples of other techniques that can be used for probe-based genotyping include, but are not limited to, AMPLIFLUOR™ nucleic acid probe technology, Dye Binding-Intercalation, Fluorescence Resonance Energy Transfer (FRET), Hybridization Signal Amplification Method (HSAM), HYBPROBE™ nucleic acid probe technology, Invader/Cleavase Technology (Invader/CFLP™ nucleic acid probe technology), MOLECULAR BEACONS™ nucleic acid probe technology, ORIGEN™ nucleic acid probe technology, DNA-Based Ramification Amplification technology, Rolling circle amplification technology (RCAT™ nucleic acid detection system), SCORPIONS™ nucleic acid probe technology, and Strand displacement amplification (SDA).

The assay formats described above typically utilize labeled oligonucleotides to facilitate detection of the hybrid duplexes. Oligonucleotides can be labeled by incorporating a label detectable by spectroscopic, photochemical, biochemical, immunochemical, radiological, radiochemical or chemical means. Useful labels include ³²P, fluorescent dyes, electron-dense reagents, enzymes (as commonly used in ELISAs), biotin, or haptens and proteins for which antisera or monoclonal antibodies are available. Labeled oligonucleotides of the disclosure can be synthesized and labeled using the techniques described above for synthesizing oligonucleotides. For example, a dot-blot assay can be carried out using probes labeled with biotin, as described in Levenson et al., 1989, in PCR Protocols: A Guide to Methods and Applications (Innis et al., eds., Academic Press. San Diego), pages 99 112, incorporated herein by reference. Following hybridization of the immobilized target DNA with the biotinylated probes under sequence-specific conditions, probes which remain bound are detected by first binding the biotin to avidin-horseradish peroxidase (A-HRP) or streptavidin-horseradish peroxidase (SA-HRP), which is then detected by carrying out a reaction in which the HRP catalyzes a color change of a chromogen.

Whatever the method for determining which oligonucleotides of the disclosure selectively hybridize to MAOA, TPH2 or DRD2 allelic sequences in a sample, the central feature of the typing method involves the identification of the MAOA, TPH2 or DRD2 alleles present in the sample by detecting the variant sequences present.

Linkage disequilibrium is the non-random association of alleles at two or more loci and represents a powerful tool for mapping genes involved in disease traits. Biallelic markers, because they are densely spaced in the human genome and can be genotyped in more numerous numbers than other types of genetic markers, are particularly useful in genetic analysis based on linkage disequilibrium.

A number of methods can be used to calculate linkage disequilibrium between any two genetic positions, in practice linkage disequilibrium is measured by applying a statistical association test to haplotype data taken from a population.

While direct haplotyping of both copies of the gene can be performed with each copy of the gene analyzed independently, it is also envisioned that direct haplotyping could be performed simultaneously if the two copies are labeled with different tags, or are otherwise separately distinguishable or identifiable. For example, if first and second copies of the gene are labeled with different first and second fluorescent dyes, respectively, and an allele-specific oligonucleotide labeled with yet a third different fluorescent dye is used to assay the polymorphism(s), then detecting a combination of the first and third dyes would identify the polymorphism in the first gene copy while detecting a combination of the second and third dyes would identify the polymorphism in the second gene copy.

In both the direct and indirect haplotyping methods, the identity of a nucleotide (or nucleotide pair) at a polymorphic site (PS) in the amplified target region may be determined by sequencing the amplified region(s) using conventional methods. If both copies of the gene are represented in the amplified target, it will be readily appreciated by the skilled artisan that only one nucleotide will be detected at a PS in individuals who are homozygous at that site, while two different nucleotides will be detected if the individual is heterozygous for that site. The polymorphism may be identified directly, known as positive-type identification, or by inference, referred to as negative-type identification. For example, where a polymorphism is known to be guanine and cytosine in a reference population, a site may be positively determined to be either guanine or cytosine for an individual homozygous at that site, or both guanine and cytosine, if the individual is heterozygous at that site. Alternatively, the site may be negatively determined to be not guanine (and thus cytosine/cytosine) or not cytosine (and thus guanine/guanine).

Once a first biallelic marker has been identified in a genomic region of interest, the practitioner of ordinary skill in the art, using the teachings of the disclosure, can easily identify additional biallelic markers in linkage disequilibrium with this first marker. As mentioned before, any marker in linkage disequilibrium with a first marker associated with a trait will be associated with the trait. Therefore, once an association has been demonstrated between a given biallelic marker and a trait, the discovery of additional biallelic markers associated with this trait is of interest in order to increase the density of biallelic markers in this particular region. The causal gene or mutation will be found in the vicinity of the marker or set of markers showing the highest correlation with the trait.

Identification of additional markers in linkage disequilibrium with a given marker involves: (a) amplifying a genomic fragment comprising a first biallelic marker from a plurality of individuals; (b) identifying of second biallelic markers in the genomic region harboring said first biallelic marker; (c) conducting a linkage disequilibrium analysis between said first biallelic marker and second biallelic markers; and (d) selecting said second biallelic markers as being in linkage disequilibrium with said first marker. Subcombinations comprising steps (b) and (c) are also contemplated.

Kits

The present disclosure also relates to a kit, a container unit comprising useful components for practicing the present method. A useful kit can contain oligonucleotide probes specific for MAOA, TPH2 or DRD2 alleles. The kit can also include instructions for correlating the assay results with the subject's risk for having or developing a mental disorder, the subject's prognostic outcome for the mental disorder, or the probability of success or failure of a particular drug treatment in the subject.

In some cases, detection probes may be fixed to an appropriate support membrane. The kit can also contain amplification primers for amplifying regions of the MAOA, TPH2 or DRD2 loci encompassing the polymorphic sites, as such primers are useful in embodiments of the disclosure. Alternatively, useful kits can contain a set of primers comprising an allele-specific primer for the specific amplification of MAOA, TPH2 or DRD2 alleles. Other optional components of the kits include additional reagents used in the genotyping methods as described herein. For example, a kit additionally can contain an agent to catalyze the synthesis of primer extension products, substrate nucleoside triphosphates, reagents for labeling and/or detecting nucleic acid (for example, an avidin-enzyme conjugate and enzyme substrate and chromogen if the label is biotin) and appropriate buffers for amplification or hybridization reactions.

The present disclosure also relates to an array, a support with immobilized oligonucleotides useful for practicing the present method. A useful array can contain oligonucleotide probes specific for MAOA, TPH2 or DRD2 alleles or certain combinations of MAOA, TPH2 or DRD2 alleles. The oligonucleotides can be immobilized on a substrate, e.g., a membrane or glass. The oligonucleotides can, but need not, be labeled. In some embodiments, the array can be a micro-array. In some embodiments, the array can comprise one or more oligonucleotides used to detect the presence of two or more MAOA, TPH2 or DRD2 alleles or certain combinations of MAOA, TPH2 and/or DRD2 alleles.

The disclosure also features diagnostics and prognostics that include identifying the allelic status of one or more SNPs (or biomarkers) which is associated with the risk for development, diagnosis, treatment, prognosis, or differentiation of a mental disorder. Once such SNP(s) are identified, the allelic pattern of such SNPs in a patient sample can be measured. These biomarkers can then be compared to a reference pattern determined by an algorithm that is associated with the risk for development, diagnosis, treatment, prognosis, or differentiation of a mental disorder. By correlating the patient pattern to the reference pattern, the presence or absence of a risk for developing a mental disorder, the presence of a mental disorder, the prognostic outcome of the mental disorder, and the probability of treatment outcomes in a patient may be determined.

In certain embodiments, a polymorphism is correlated to a condition or disease by merely its presence or absence. In other embodiments, an algorithm is needed to relate the pattern of biomarkers to a desired prediction outcome in the subject. Algorithmic techniques for relating biomarkers of the present disclosure include a linear regression technique, a nonlinear regression technique, an ANOVA technique, a neural network technique, a genetic algorithm technique, a support vector machine technique, a tree learning technique, a nonparametric statistical technique, a forward, backward, and/or forward-backward technique, and a Bayesian technique. The skilled artisan will recognize the word “technique” refers to a process in which a predictor is built by using patient exemplar pairs of biomarkers and phenotypes, and then refining such predictor algorithm in an iterative process by testing a version of the algorithm on unseen data and making changes to mathematical coefficients of such algorithm in such a way to increase the accuracy and specificity of the predictor algorithm.

In other embodiments, the disclosure relates to methods for determining a treatment regimen for use in a subject diagnosed with a mental disorder. The methods may comprise determining the presence of one or more biomarkers as described herein, and using the biomarkers to refine a diagnosis for a subject. One or more treatment regimens that improve the subject's prognosis by reducing the increased disposition for an adverse outcome associated with the diagnosis can then be used to treat the subject. Such methods may also be used to screen pharmacological compounds for agents capable of improving the subject's prognosis as above.

Use of Functional Polymorphisms to Screen for Drugs

The disclosure also relates to screening methods using animal models of drug responsiveness to identify the effect of the biomarkers on the animal's response to drug therapy.

For example, in one aspect, experimental animals can be genetically engineered to carry one or more functional SNPs or haplotypes, or SNP's in linkage disequilibrium with the functional SNP's or haplotypes (knock-in technology). Then, the knock-in animal's response to drug therapy can be compared to control animals to determine changes in drug response. The alteration of the animal's drug response as a result of the presence of the functional polymorphism can then be used to construct a reference pattern of biomarkers associated with drug response.

In one aspect, the animal is a non-human primate, a mammal, or a mouse.

Any suitable test compound may be used with the screening methods of the disclosure. Examples of compounds that may be screened by the methods of the disclosure include small organic or inorganic molecules, nucleic acids (e.g., ribozymes, antisense molecules), including polynucleotides from random and directed polynucleotide libraries, peptides, including peptides derived from random and directed peptide libraries, soluble peptides, fusion peptides, and phosphopeptides, antibodies including polyclonal, monoclonal, chimeric, humanized, and anti-idiotypic antibodies, and single chain antibodies, FAb, F(ab′)₂ and FAb expression library fragments, and epitope-binding fragments thereof. In certain aspects, a test compound for treating a mental disorder may include, by way of example, antipsychotic drugs in general, neuroleptics, atypical neuroleptics, antidepressants, anti-anxiety drugs, noradrenergic agonists and antagonists, dopaminergic agonists and antagonists, serotonin reuptake inhibitors, benzodiazepines.

The examples of the present disclosure presented below are provided only for illustrative purposes and not to limit the scope of the disclosure. Numerous embodiments of the disclosure within the scope of the claims that follow the examples will be apparent to those of ordinary skill in the art from reading the foregoing text and following examples.

### Example 1

### Allelic mRNA Expression of X-Linked MAOA in Human Brain

To explore the effect of polymorphisms and epigenetic factors on mRNA expression, we have measured allelic expression imbalance (AEI) in female human brain tissue, employing two frequent marker SNPs in exon 8 (T890G) and exon 14 (C1409T) of MAOA. This approach compares one allele against the other in the same subject. AEI ratios ranged from 0.3 to 4 in prefrontal cortex, demonstrating the presence of strong cis-acting factors in mRNA expression. Analysis of CpG methylation in the MAOA promoter region revealed substantial methylation in females but not males. MAOA methylation ratios for the 3- and 4-repeat pVNTR alleles of MAOA did not correlate with X chromosome inactivation ratios, determined at the X-linked androgen receptor locus, suggesting an alternative process of dosage compensation in females. The extent of allelic MAOA methylation was highly variable and correlated with AEI (R²=0.5 and 0.7 at two CpG loci), indicating that CpG methylation regulates gene expression. Genetic factors appeared also to contribute to the AEI ratios. Genotyping of 13 MAOA polymorphisms in female subjects showed strong association with a haplotype block spanning from the pVNTR to the marker SNP. Therefore, allelic mRNA expression is affected by genetic and epigenetic events, both with the potential to modulate biogenic amine tone in the CNS.

Introduction: Genetic predisposition to mental disorders appears to involve multiple genes, but a causative relationship has been difficult to establish. Extensive studies on suspected disease susceptibility genes have focused on functional polymorphisms that change the encoded amino acid sequence. However, polymorphisms in regulatory regions, or those affecting mRNA processing, also affect clinical phenotypes. Recent genome-wide surveys suggest that these cis-acting polymorphisms might account for much of human phenotypic diversity. Yet, a systematic analysis of the prevalence and impact of cis-acting regulatory polymorphisms has yet to be performed for most susceptibility genes implicated in mental disorders.

This study focuses on monoamine oxidase A (MAOA), a candidate gene implicated in multiple CNS disorders, such drug abuse, aggression, antisocial behavior, anxiety, attention deficit hyperactivity disorder, anorexia nervosa, bipolar disorder and Alzheimer's disease. Monoamine oxidases catalyze the oxidation of biogenic amines and are the target of a class of antidepressant drugs. A repeat polymorphism in the promoter region of MAOA (pVNTR) has been extensively studied in vitro and in clinical association studies. The 4 repeat pVNTR yielded higher expression levels of a reporter gene than the 3 repeat, in a heterologous in vitro system. On the basis of this result, the pVNTR of MAOA has been a marker for numerous association studies, suggesting a link to increased susceptibility to impulsivity and early abuse experiences in males, while other studies have failed to demonstrate significant associations with various disorders. The 3-repeat pVNTR variant also influences aggressive behavior in Rhesus monkeys. Moreover, MAOA knockout mice display offensive aggressive behavior in males. Yet, the functional relevance of the pVNTR and its contribution to overall genetic diversity of MAOA in the CNS has yet to be demonstrated.

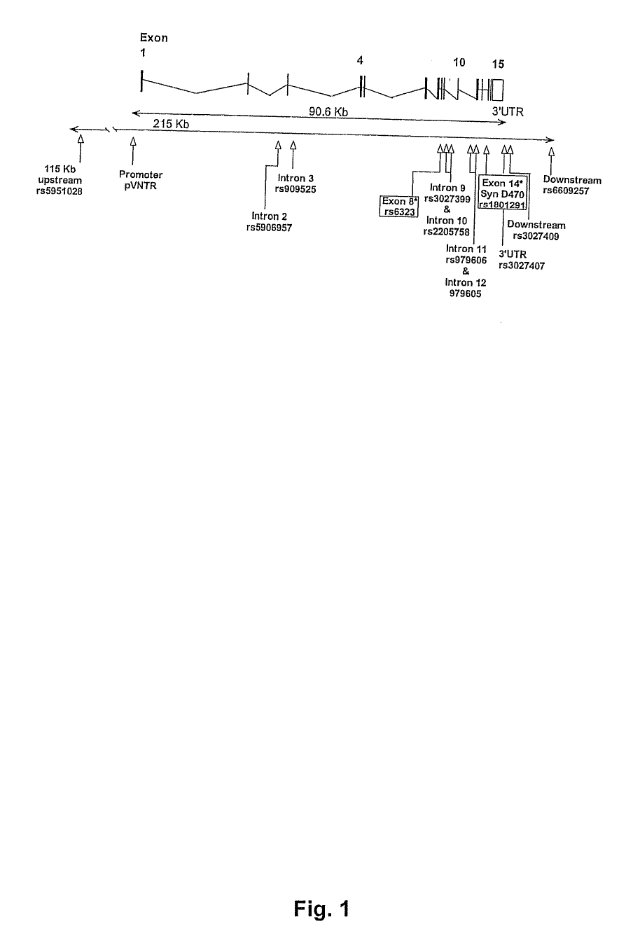

Epigenetic factors provide an alternative mode of gene regulation. MAOA is located on the X chromosome at Xp11.3, adjacent to the MAOB gene. The MAOA gene spans at least 90.6 kilobases (Kb) from the promoter to the 3′ untranslated region (FIG. 1). While one commonly assumes that in each cell one X-chromosome is randomly inactivated in females, unequal X inactivation or selection of one active X-chromosome over the other in somatic cells has been observed. Earlier reports had suggested that MAOA is subject to X chromosome inactivation in humans. However, a recent survey of X inactivation found that MAOA ranks among the 15% of X-linked genes that escape inactivation. Therefore, the contribution of X-inactivation or other epigenetic factors to regulation of MAOA remain unexplored.

This study addresses the question how genetic and epigenetic processes interact to regulate MAOA gene expression using human autopsies from brain tissues. We address this question by quantitatively measuring the relative amounts of mRNA generated from each of the two alleles in female subjects, using two marker SNPs in the transcribed region. An allelic expression imbalance (AEI) indicates the presence of cis-acting factors in gene regulation and/or mRNA processing. Analysis of AEI has been successfully applied in several recent studies, including those involving brain tissues (Bray, N. J., et al. (2003) Hum. Genet., 113, 149-153, 4, 38-40; Johnson, A., et al. (2005) Pharmacol. Ther., 106, 19-38; Pastinen, T. and Hudson, T. (2004) Science, 306, 647-650; Zhang, Y., et al. (2005) J. Biol. Chem., 280, 32618-32624; Lim, J E, et al. (2006) Mol Psychiatry, July; 11 (7):649-62. Epub 2006 Jan. 24) each incorporated herein by reference. AEI results provide a quantitative measure of the allelic differences in each individual, one allele serving as the control for the other, while canceling out any trans-acting factors. It also enables scanning a gene for functional polymorphisms, using AEI as a phenotype as previously demonstrated for MDR1 (Zhang, Y., et al. (2005) J. Biol. Chem., 280, 32618-32624) and OPRM1 (Wang, D., et al. (2005) Pharmacogenet. Genomics, 15, 693-704) each incorporated herein by reference. This is the first study to exploit AEI as a quantitative phenotype for dissecting the contribution of genetic and epigenetic factors to interindividual variability.

Measuring allelic mRNA expression compares one allele against the other in a relevant autopsy target tissue of the same individual—females in the case of X-linked genes. Allelic expression ratios appear to represent a more robust phenotypic marker than absolute mRNA levels, which can fluctuate strongly because of trans-acting factors and post-mortem decay. To survey diverse MAOA alleles that may be enriched in disease, we have included control subjects, and those previously diagnosed with schizophrenia and bipolar disorder. We have analyzed autopsy brain samples from 105 individuals (36 females and 69 males) previously diagnosed with bipolar disorder (35) or depression (35), and 35 controls, obtained from the Stanley Foundation. While the number of female subjects in this study was sufficient for detection and evaluation of cis-acting factors by pairwise allele comparisons, the size of the cohorts was not designed to permit a robust association analysis in a case-control study design. Brain tissues were taken from prefrontal cortex, and in 4 cases from three other brain regions as well. All samples were genotyped for 13 common polymorphisms, two of which served as marker SNPs in the transcribed region for analyzing AEI. In addition, we measured total mRNA levels in all samples. To account for epigenetic effects, we determined CpG island methylation in the MAOA promoter region in two loci, in comparison to X inactivation measured at the X-linked androgen receptor locus. In the present study, the male samples served in assigning unambiguous haplotypes, and to compare CpG methylation between males and females. The results reveal epigenetic gene regulation by CpG methylation in the MAOA promoter region in females (but not males) representing a possible dosage compensation mechanism that does not correlate with X inactivation. After accounting for epigenetic factors, one or more cis-acting polymorphisms also affect allelic mRNA levels. The functional variant locates to an MAOA haplotype region spanning from the pVNTR in the promoter to the 3′ end of MAOA.

Results

Genotype and Haplotype Analysis of MAOA

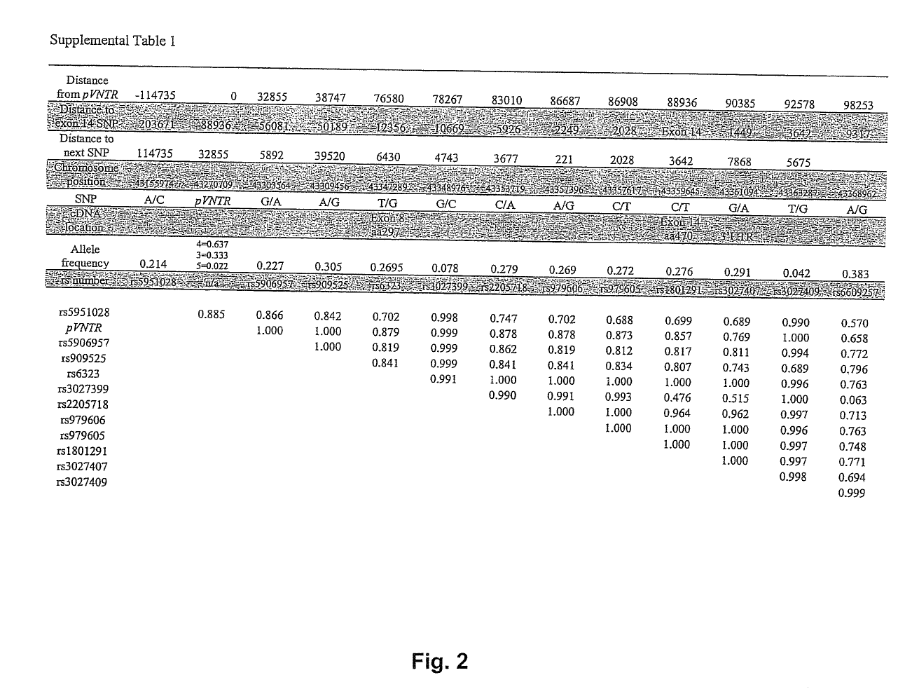

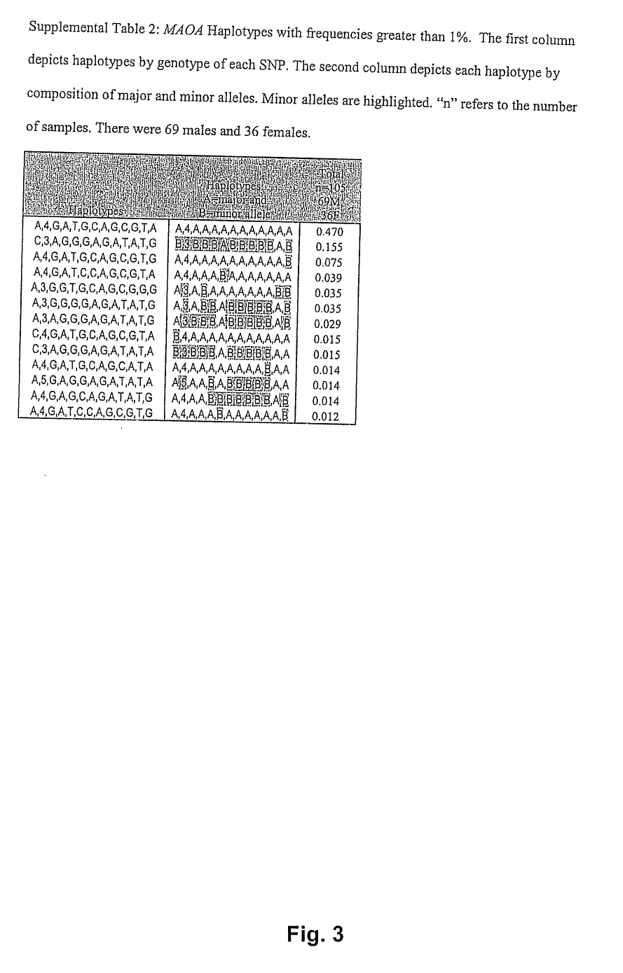

We genotyped 13 polymorphisms, spanning the MAOA gene (FIG. 1) in 105 samples (69 male, 36 female) from the Stanley foundation brain collection, including the promoter variable nucleotide tandem repeat (pVNTR). Allele frequencies, linkage, and other information about each polymorphism (shown in FIG. 2), are consistent with previous results (The International HapMap Consortium (2003) The International HapMap Project. Nature, 426, 789-796). We identified 14 unambiguous haplotypes in the males, carrying only a single X chromosome. Through the use of an estimation maximization algorithm to assess haplotypes and their frequencies including males and females, we identified 10 additional haplotypes for a total of 23. Haplotype information is depicted in FIG. 3. The haplotype block extends at least 115 Kb upstream from the MAOA locus (The International HapMap Consortium (2003) The International HapMap Project. Nature, 426, 789-796) incorporated herein by reference. Downstream of MAOA, the haplotype block ends approximately 10 Kb from the 3′ end. Pair-wise linkage disequilibrium results (FIG. 2) are consistent with these data. A haplotype block of 6 abundant (>30% allele frequency) SNPs in very high linkage disequilibrium spreads over the 3′ portion of MAOA. This includes 3 high frequency SNPs in transcribed regions (exon 8 and 14 and 3′UTR), from which we have selected the exon 8 and 14 SNPs as markers for AEI assays. These two marker SNPs are linked to each other in all but one individual. In the majority of samples with a 4-repeat pVNTR, the 4-repeat is linked to the major alleles, and the 3-repeat to the minor ones, of the two indicator SNPs in exon 8 and 14, with 4 notable exceptions. The latter are important for assessing those gene regions that might contribute to allelic expression imbalance.

Allele-Specific mRNA Analysis

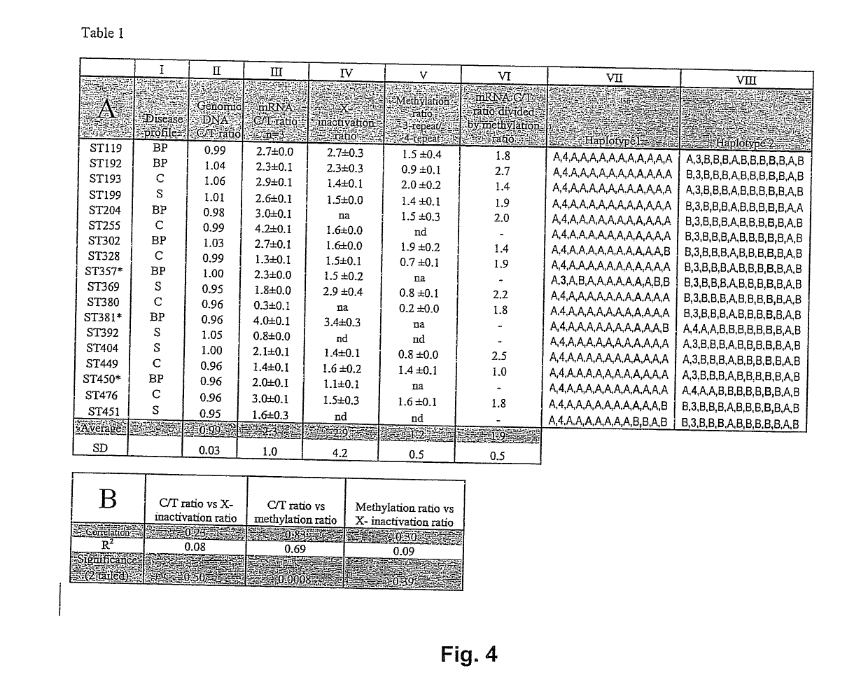

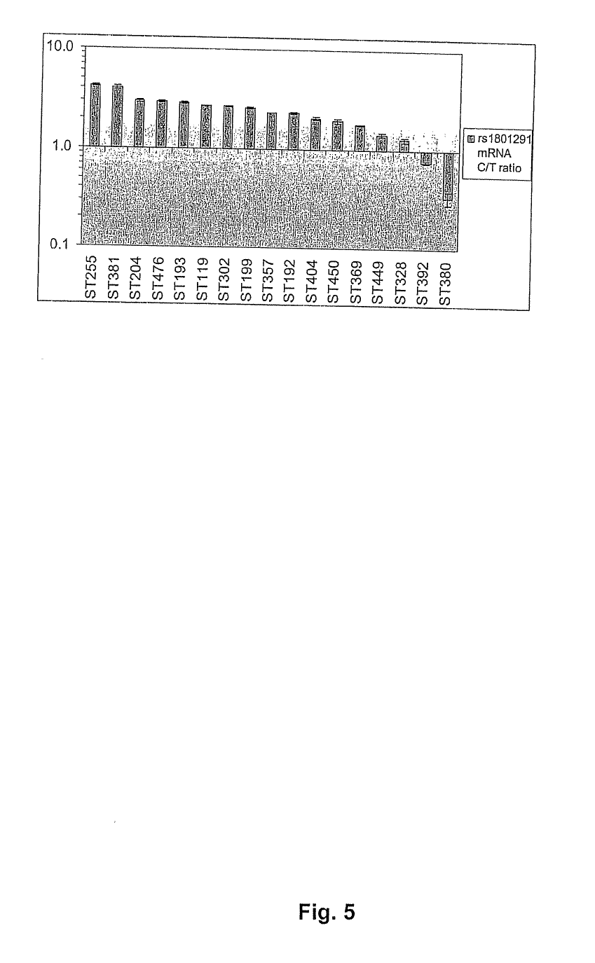

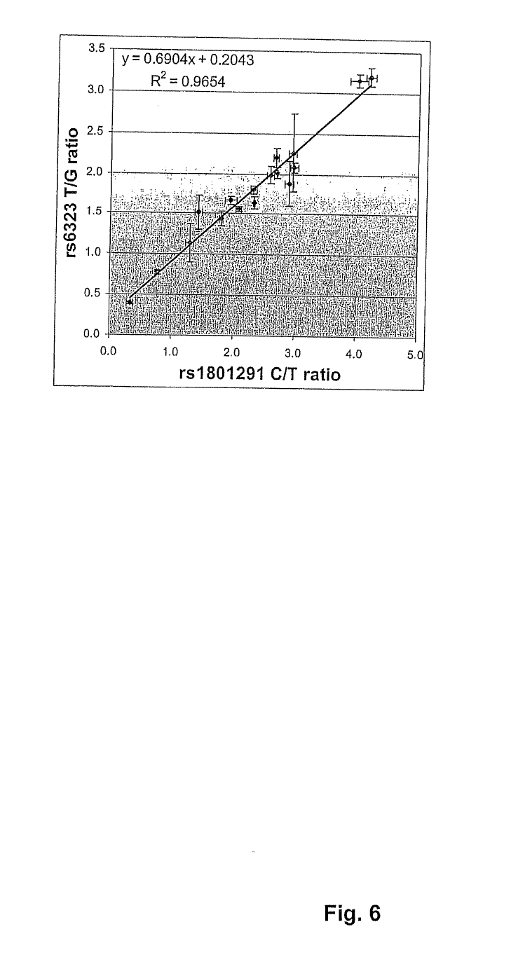

We next measured the ratios of MAOA genomic DNA alleles in comparison to the corresponding allelic mRNA ratios, in prefrontal cortex samples. Any significant difference in these ratios documents the presence of AEI, and hence cis-acting factors determining mRNA levels. From the available genotype data, we have first selected the synonymous C/T SNP (C1409T) in exon 14 (rs1801291) as a marker for the AEI analysis. Among 36 female DNA samples from the Stanley Foundation brain collection, for which mRNA from the prefrontal cortex was available, 17 samples were heterozygous for the marker SNP and therefore suitable for AEI analysis. This enables evaluation of functional differences for 34 chromosomes. These included 6 controls, 7 bipolar patients and 5 schizophrenic patients. The genomic DNA ratios varied within a narrow range and were normalized to 1.0 (S.D.=0.03) (FIG. 4, column II), showing the excellent reproducibility of the DNA ratio analysis, even in extracted brain autopsy samples. FIG. 5 shows a plot of the mRNA C/T ratios derived from measurements of each allele and normalized to a genomic ratio of 1. The intra-sample error of repeat analysis was higher for mRNA ratios than for DNA ratios, owing to RNA degradation. Nevertheless, AEI ratios deviating from unity by >25% are detectable. In all but 2 samples, the C allele (major allele) was expressed at a higher level than the T allele (minor allele). In the other two samples, the expression pattern was reversed, with the T allele expressed at a higher level. The C/T ratios varied from 0.33±0.05 to 4.2±0.1, revealing a substantial expression imbalance of an order likely to have physiological relevance. Three female subjects were heterozygous for the marker SNP but homozygous for the pVNTR (FIG. 4, columns VII and VIII). Nevertheless, these samples displayed significant AEI values, indicating that the pVNTR is not affecting these ratios but other or additional factors are present. These data were validated by repeating the AEI analysis with a second synonymous SNP in exon 8 (rs6323) yielding similar results as shown in FIG. 6 (Pearson correlation=0.98). The high correlation between the two independent AEI assays validates the allelic ratio analysis. Furthermore, we obtained AEI data for an additional sample heterozygous for rs6323 (ST451) which is homozygous for rs1801291 but, yet displays significant AEI (AEI ratio=1.6±0.3). In this case, rs1801291 cannot have been the cause of the AEI ratio.

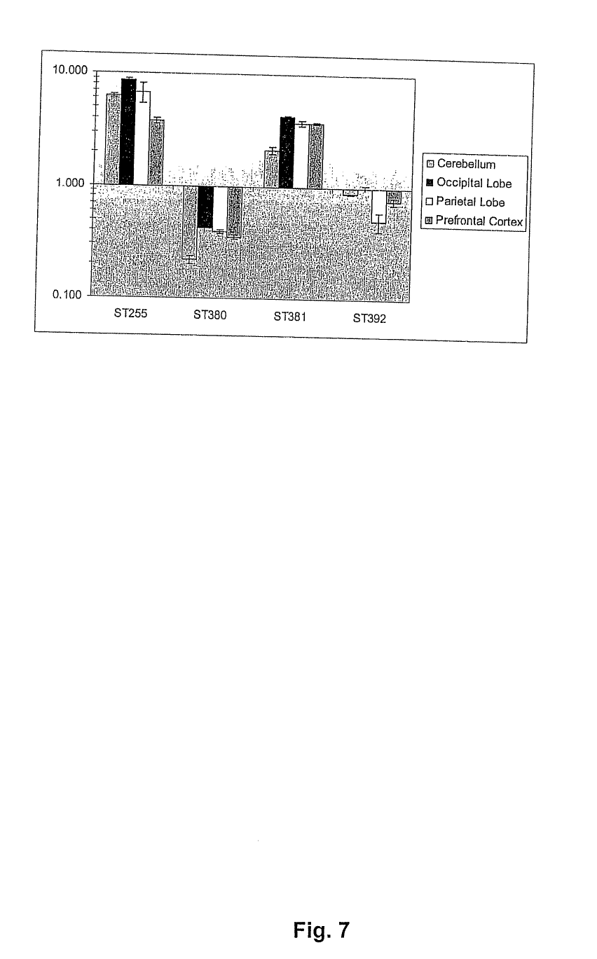

To explore the possibility of tissue-specific differences in allelic expression, we analyzed allele-specific mRNA ratios from different brain regions: cerebellum, occipital lobe, and parietal lobe, from 4 individuals from the Stanley Foundation collection. Samples ST255 and ST381 had the highest C/T ratios (4.2±0.1 and 4.0±0.1, respectively), while samples ST380 and ST392 had the lowest ratios (0.33±0.05 and 0.77±0.02, respectively). Shown in FIG. 7, there is some variability in the allelic mRNA expression ratios from tissue to tissue in the same individual that could be due to tissue-specific factors or sample quality. However, the overall trend in different tissues across individuals remains the same. The two individuals with high C/T ratios in prefrontal cortex maintained consistently high C/T ratios in the other brain regions, and the two individuals with low C/T ratios in prefrontal cortex also had lower C/T ratios in other brain regions.

MAOA Methylation of a Promoter CpG Island in Comparison to X Chromosome Inactivation Measured at the Androgen Receptor Locus

We next performed a set of experiments to assess methylation in the CpG island located within the promoter region of MAOA, in comparison to X-inactivation. We determined allelic methylation ratios of the 3- and 4-repeat alleles of MAOA in females, using the methylation-sensitive restriction enzymes HhaI and SmaI. A first assay relied on simultaneous amplification of 3- and 4-repeat alleles of the pVNTR, after digestion with Sma I, yielding a set of allelic methylation ratios for 15 females for each the 3- and 4-repeat alleles. CpG methylation prevents digestion, revealing undigested 3 and 4 repeat amplicons. Ratios of 3-repeat over 4-repeat methylation, listed in Table 1, column V, varied over a tenfold range. X-inactivation ratios were obtained by measuring CpG island methylation in the polyallelic promoter region of the androgen receptor (Sandovici, I., et al. (2004) Hum. Genet., 115, 387-392) incorporated herein by reference. Methylation at the androgen receptor locus has been shown to correlate with inactivation of the X chromosome (Allen, R., et al. (1992) Am. J. Hum. Genet., 51, 1229-1239) incorporated herein by reference.

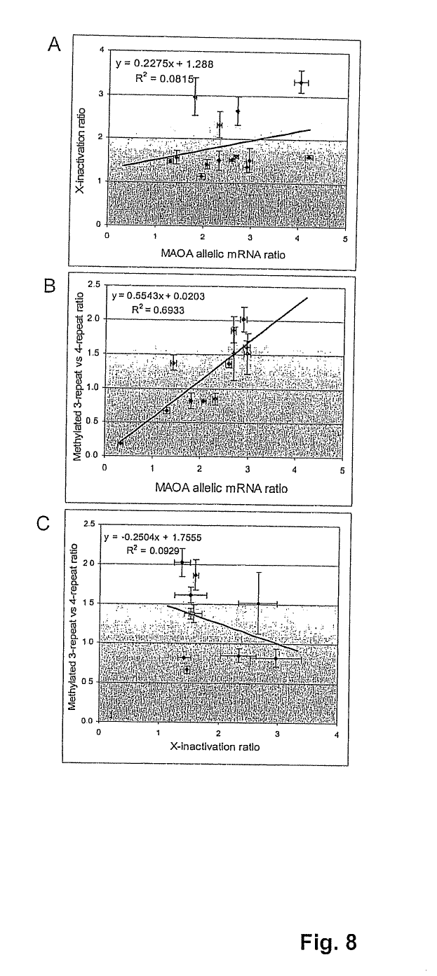

FIG. 8A depicts a comparison between the allelic methylation ratios of the androgen receptor gene—a measure of unequal X inactivation—against the MAOA allelic mRNA expression ratios, determined with HhaI. The low concordance (Pearson correlation r=0.29, p=0.50), combined with previous findings that MAOA escapes X inactivation (Carrel, L. and Willard, H. (2005) Nature, 434, 400-404) indicates that the observed AEI of MAOA is independent of unequal X-inactivation. Allelic methylation ratios obtained with SmaI also correlated poorly with X-inactivation (r=0.05).

MAOA CpG Methylation in Comparison to AEI

We next compared allele-specific CpG methylation with AEI ratios in samples heterozygous for both the marker SNPs and the pVNTR. Heterozygosity in the latter was needed because the methylation assay exploited the 3- and 4-repeats to distinguish between alleles. If CpG methylation of the MAOA promoter affects transcription, differences in methylation between alleles should result in AEI ratios distinct from unity. Indeed, when the MAOA 3-/4-repeat methylation ratios are plotted against the AEI ratios (FIG. 8B), a robust correlation is revealed (Pearson correlation r=0.83, p=0.0008 for Sma I; Pearson correlation r=0.73, p=0.004 for Hha I). This result indicates that methylation affects transcription and could account for 50-70% of the observed AEI ratios; however, other factors appear also to play a role and likely involve genetic polymorphisms.

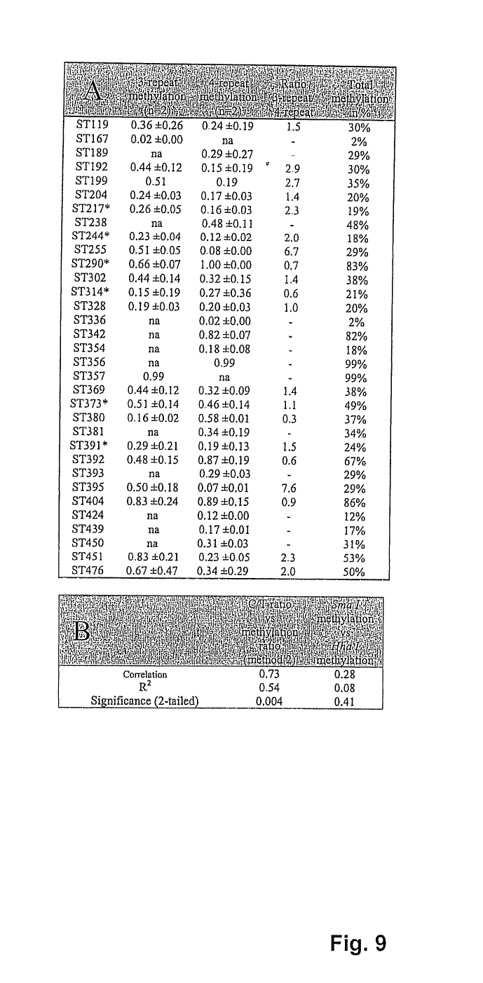

The pVNTR itself contains CpG islands and is contiguous with the main CpG island of the MAOA promoter. Therefore, relative allelic methylation could have varied with the number of repeats in the pVNTR. Allele-specific methylation ratios in 6 females heterozygous for the pVNTR and homozygous for the marker SNP were determined from percent methylation of each allele (indicated by * in FIG. 9A). These ratios were distributed randomly and failed to correlate with the pVNTR genotype (data not shown). Therefore, CpG methylation appears to be independent of pVNTR genotype.

Measuring the ratio of methylated alleles does not provide information on the overall extent of methylation at the MAOA locus. Therefore, we analyzed the fraction of CpG methylation for each MAOA allele separately, using a newly developed method with Hha I (FIG. 9A). Of 12 male tissues analyzed, no significant methylation was detectable at the MAOA locus (data not shown). In contrast, the 35 female samples analyzed displayed variable levels of total methylation of both alleles, ranging from 2% to near complete methylation (mean of the two alleles). This is incompatible with a mechanism of X inactivation, where one would expect a mean of 50% methylation between the two alleles in female tissues. Analysis of females heterozygous for the 3- and 4-repeat pVNTR again revealed variable extent of methylation for each of the two alleles.

Association of MAOA Genotypes with Allelic Expression Imbalance

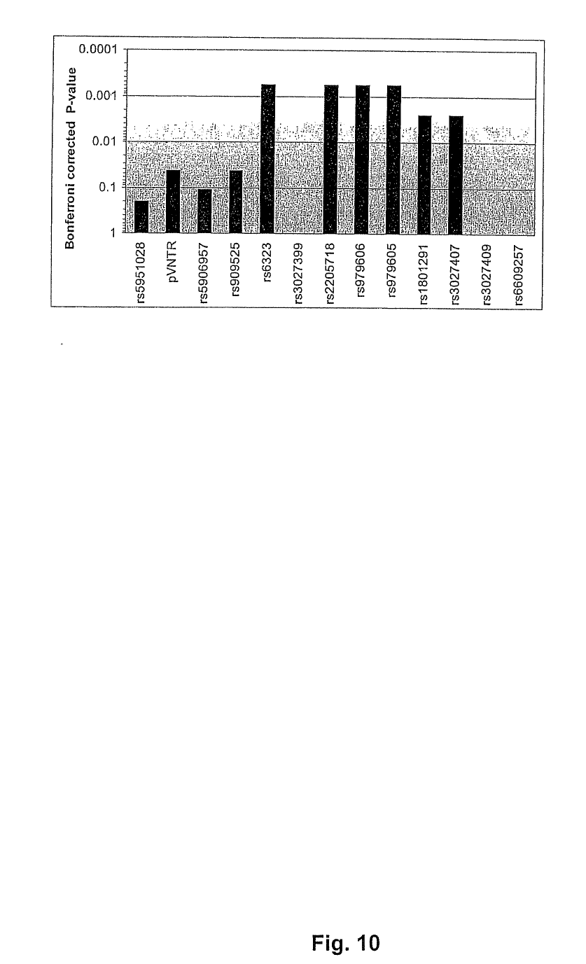

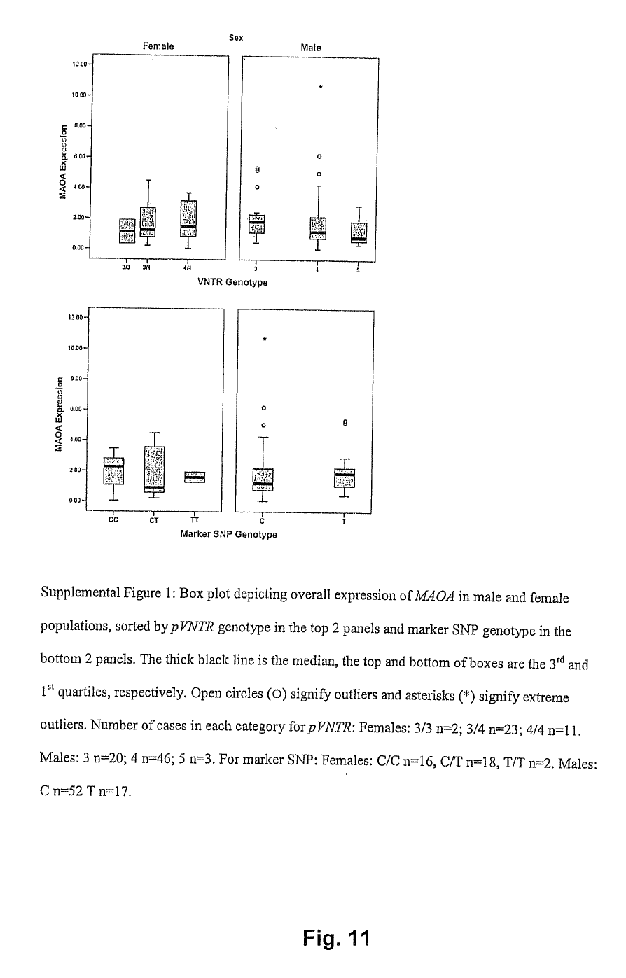

Since promoter methylation cannot fully account for the observed AEI ratios, we used the allelic expression ratios shown in FIG. 4 as the phenotype to scan the MAOA gene locus for regions containing associated polymorphisms. If cis-acting polymorphisms contribute to the measured AEI ratios—in addition to epigenetic factors—significant correlations should be detectable. For this analysis it is helpful to know the phasing of each SNP and the pVNTR with the marker SNPs. Phasing between two polymorphisms can be ambiguous. For MAOA however, accurate assignment of the haplotypes (inferred from all male and females samples) enabled us to relate allelic expression ratios (at the marker SNP) directly to the corresponding haplotypes for each of the female samples assayed for AEI (FIG. 4, columns VII and VIII). On the basis of these results, we conducted a single locus association test between SNP genotype and allelic expression in the female samples (FIG. 10). Alleles of each SNP were sorted according to whether they were found on the high or low expressing allele in the allele-specific mRNA analysis. The significance of the contribution of each SNP towards the high or low phenotype was determined. Four SNPs: rs6323 (exon 8), rs2205718, rs979606 and rs979605 were significantly associated with expression level, with Bonferroni corrected p-values less than 0.001. rs1801291 (the marker in exon 14) and rs3027407 (3′UTR) had p-values<0.01, while the pVNTR and rs909525 were less strongly but still significantly associated with AEI (Bonferroni corrected p-value<0.05). These significant associations indicate that genetic factors also contribute to the observed AEI, by affecting mRNA expression levels.

The highly significant association of the block of 4 SNPs between the exon 14 marker SNP and pVNTR strongly suggests that this region harbors a genetic variant contributing to AEI; however, unequal allelic methylation could confound this interpretation. The 3 samples homozygous for the pVNTR showing significant AEI (see FIG. 4) support the notion that the functional SNP is placed elsewhere. However, these 3 samples did show some degree of overall methylation (see FIG. 9); therefore, we cannot entirely exclude the possibility that these 3 AEI ratios were generated by unequal CpG methylation between the two alleles, in each case favoring expression form the major allele. We were unable to measure this because the allele-specific methylation assay depends on heterozygosity in the pVNTR.

The quantitative nature of AEI ratios as an immediate phenotype enables an estimate of the relative contributions of a genetic polymorphism to the observed AEI, compared to epigenetic factors in females. Assuming that MAOA methylation inhibits or interferes with transcription, we can account for the contribution of allelic methylation before linking the AEI to any underlying polymorphisms. FIG. 4, column VI shows the allelic mRNA ratios adjusted for methylation. Adjusted values were derived by dividing the mRNA C/T ratios by the methylation 3-repeat/4-repeat ratios, approximating the contribution of a causative polymorphism toward AEI. The adjusted ratios indicate that the C allele is expressed ˜1.9 fold higher than the T allele. This result strongly indicates that both epigenetic and genetic cis-acting factors are operative. Remarkably, methylation appears to account for at least one of the two samples with low C/T AEI ratios (FIG. 4).

Relationship of Overall mRNA Levels to CpG Methylation and Genotype

We measured total MAOA mRNA levels (relative to β-actin mRNA as the control) in all 105 samples, male and female. Overall expression of MAOA mRNA was high, but fluctuated over a broad range. For males, the range in arbitrary units was: 0.06 to 10.8; for females: 0.06 to 4.5) while cycle thresholds for β-actin mRNA varied much less (˜3 cycles or 8 fold). However, no association was detectable between MAOA mRNA levels and genotype and/or CpG methylation status (FIG. 11). Possibly, any genetic influence (estimated from the AEI data to be ˜1.9-fold) on overall mRNA levels was too small relative to the large mRNA variability to yield significant associations. On the other hand, some samples were nearly fully methylated in the promoter region. If CpG methylation would have completely suppressed transcription, we would expect a robust correlation between methylation and mRNA levels. Since this was not the case (R²=0.06), we propose that CpG methylation of the MAOA promoter does not abolish but modulates transcription, detectable only with the more sensitive AEI ratio measurements. Therefore, the large variation of mRNA levels was caused by trans-acting factors, or post mortem degradation, or both.

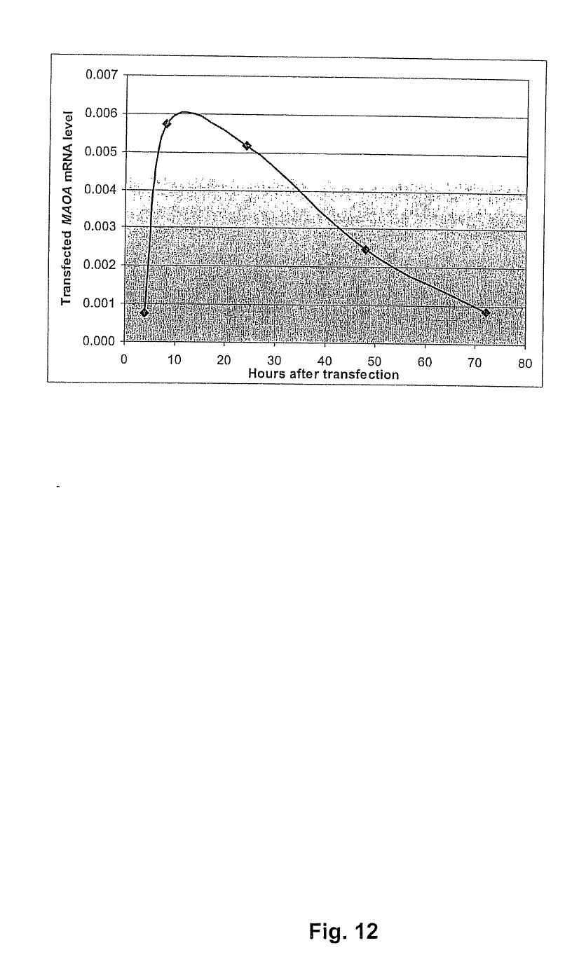

In Vitro Analysis of Transfected MAOA cDNA Variants

Whereas in vitro reporter gene assays suggest that the pVNTR affects MAOA expression (Sabol, S., et al. (1998) Hum. Genet., 103, 273-279) incorporated herein by reference, our genotype-AEI association analysis favored a region in the 3′ portion of MAOA. Since the marker SNP itself (rs1801291) and two other SNPs (rs6323 and rs3027407) located in the cDNA are highly linked to AEI, those loci were promising candidates. We prepared human MAOA cDNA constructs containing the 3 SNPs together, as well as each SNP individually, and transfected each one along with the wild-type allele construct in cultured Chinese hamster ovary cells (hamster MAOA DNA and mRNA did not interfere with our assays). mRNA expression peaked at approximately 10 hours after transfection and then declined over the next 2-3 days (FIG. 12). Mutant cDNA constructs were cotransfected with wild type MAOA under the promoter of the vector, and plasmid DNA ratios and mRNA (after conversion to cDNA) ratios were determined at various time points (8, 24 and 48 hours). DNA ratios remained constant, and allelic mRNA ratios similarly did not deviate from DNA ratios at all time points measured (AEI: 8 hours 1.0±0.0, 24 hours 1.0±0.0, 48 hours 1.1±0.1). Thus, in this assay, AEI analysis failed to detect a difference in expression between any of the constructs and wild type, suggesting that none of the 3 SNPs has an effect on MAOA expression from cDNA plasmids in a heterologous cell culture system. However, this assay only looks at one aspect of expression, not at transcription or mRNA processing.

Discussion

We have dissected the genetic and epigenetic mechanisms involved in the regulation of allelic expression of MAOA, an X-linked gene implicated in multiple CNS disorders. This was performed on autopsy samples from prefrontal cortex, a brain region implicated in schizophrenia. The location of MAOA on the X chromosome simplifies the haplotype analysis, since males have only a single X chromosome. The haplotypes identified in this sample cohort are consistent with recently published or publicly available haplotype results (The International HapMap Consortium (2003) The International HapMap Project. Nature, 426, 789-796; Jansson, M., et al. (2005) MAOA haplotypes associated with thrombocyte-MAO activity. BMC Genet., 6, 46-55) each incorporated herein by reference. To identify cis-acting factors modulating gene expression and mRNA processing, we have measured allelic expression of MAOA mRNA in human brain tissues. While the analysis of mRNA levels in autopsy tissues, in particular brain, has been problematic, measuring allelic mRNA ratios appears to be more robust, under the assumption that each allele degrade at the same rate post-mortem.

The assay procedure was optimized and validated to yield precise and accurate results, building on experience with previous studies of AEI ratios in several genes expressed in brain tissues, such as hOPRM1 (see Zhang, Y., et al. (2005) Allelic expression imbalance of human mu opioid receptor (OPRM1) caused by variant A118G. J. Biol. Chem., 280, 32618-32624) and hPEPT2 (see Pinsonneault, J., et al. (2004) Genetic variants of the human H+/dipeptide transporter PEPT2: analysis of haplotype functions. J. Pharmacol. Exp. Ther., 311, 1088-1096) each incorporated herein by reference. Use of two marker SNPs provided an independent estimate of allelic mRNA ratios. The excellent agreement between the AEI ratios measured by the two assays supports the accuracy of the results. The precision with which AEI ratios can be measured enabled us in the present study to dissect epigenetic and genetic factors in mRNA expression, and provide estimates of their relative contributions.

The results demonstrate the presence of significant allele-specific differences (up to 4-fold) in mRNA expression of MAOA in females heterozygous for a marker SNP, with ratios ranging from 0.3 to 4.2. This range of AEI ratios suggested the likely presence of more than one cis-acting factor. Since a majority of ratios were >1 (15/17), indicating a greater expression from the main wild-type variant, at least one factor has to be preferentially associated with one allele over the other. With methylation apparently occurring at random between the two pVNTR alleles in females (determined in females homozygous for the marker SNPs), we surmise that a polymorphism (in strong linkage disequilibrium with the marker SNPs) accounts for the bias of AEI ratios>1.

Allelic mRNA expression can be affected by differences in regulatory factors or mRNA processing, and epigenetic events between different tissues. We acknowledge that the DNA and RNA extracts obtained from defined brain regions contain many types of neurons and glia, so that the measured AEI ratios represent only an average for the region. Similar allelic expression ratios in various brain regions from the same individual indicated that variation between brain regions are small compared to inter-individual differences. A fourfold difference in gene expression between alleles likely has physiological relevance. However, measured overall mRNA levels were too variable to permit linkage studies, while allelic expression ratios are robust because one allele serves as the control for the other in a target tissue. Therefore, the present study focuses on the mechanisms underlying differential expression from the two X chromosomes in females.

CpG Island Methylation of MAOA and Relationship to X-Inactivation and AEI

We considered the possibility that promoter methylation could have contributed to the AEI observed for MAOA. In females, regulation of >80% of genes on the X chromosome is commonly dominated by X inactivation. Unequal X inactivation could potentially cause allelic expression imbalance, which can remain constant between various tissues in the same individual. Unequal X inactivation can occur by numerous mechanisms and is a common phenomenon; if present, a majority of X-linked genes would show allelic expression imbalance. However, recent studies indicate that both MAOA and MAOB, positioned adjacent to MAOA, escape X inactivation. To test this further, we measured X inactivation ratios of 15 female samples using the androgen receptor locus. The androgen receptor X-inactivation ratios varied considerably between samples, as expected from previous results (Sharp, A., et al. (2000) Hum. Genet., 107, 343-349) incorporated herein by reference. If MAOA were to undergo methylation as part of X-inactivation, and CpG methylation would interfere with MAOA expression, we would expect the androgen receptor X-inactivation ratios to correlate with the AEI ratios of MAOA. However, these two events were not significantly correlated with each other. These results argue against skewed X-inactivation as a contributor to AEI of MAOA.

We next explored the possibility of methylation at the MAOA locus independent of, or not directly related to, X-inactivation. Gene silencing and imprinting by CpG island methylation play a general role in regulating gene expression, and moreover, can result in allelic differences in transcription. We have measured allele-specific methylation, using methylation-sensitive restriction enzymes at two sites (Sma I and Hha I), determining either allelic methylation ratios only, or additionally the overall extent of methylation for both alleles. CpG methylation of MAOA occurs exclusively in females, where it ranged from 2% to near complete methylation. Lack of correlation between the degree of MAOA methylation and overall mRNA expression in our RNA samples appears to be a result of a rather large variability in mRNA levels. Since MAOA mRNA levels are robustly expressed, even in samples with high methylation, any effect of CpG island methylation can only be partial at the most.

We also found that promoter methylation varied greatly between the 3- and 4-repeat alleles in the same individual. Since allele-specific methylation is significantly correlated with allele-specific mRNA expression (correlation with AEI ratios R=0.7 to 0.8), these findings support the hypothesis that MAOA promoter methylation modulates transcription. MAOA promoter methylation could therefore represent a mechanism of partial dosage compensation independent of X-chromosome inactivation, which however is highly variable among individuals.

Dissection of Epigenetic and Genetic Factors

We next considered whether the proposed AEI effect of a cis-acting polymorphism can be distinguished from epigenetic effects. Assuming that methylation reduces transcription, dividing the measured AEI ratios with the methylation ratios should yield a rough estimate of the effect exerted only by cis-acing polymorphism(s). The mean of the adjusted AEI ratios did not differ substantially from the mean of the measured AEI ratios—consistent with the notion that methylation is random between alleles. More importantly, the adjusted AEI ratios fall in a more narrow range (2.3±1.0 before and 1.9±0.5 after the adjustment) (FIG. 4), suggesting that this ratio reflects a potential cis-acting polymorphism more accurately. Remarkably, allelic methylation differences appeared to account for the sample with the lowest AEI ratios (0.33), owing to high methylation of the main wild-type allele. Because ratios are not linearly related to expression activity and moreover methylation appears to modulate rather than abolish transcription, these estimates are only approximations. Nevertheless, these results taken together support a contribution from genetic factors of twofold in regulating MAOA expression, which is superimposed on variable changes afforded by CpG methylation. We conclude that both genetic and epigenetic factors contribute to nearly similar extents to variable mRNA expression in females. However, in males methylation was not observed and only genetic factors could play a role. Genetic association studies need to reflect these relationships, with clear differences in gene regulation between males and females. This is consistent with large sex differences in susceptibility and presentation of mental disorders (Pinsonneault, J. and Sadee, W. (2003) AAPS PharmSci., 5, E29) incorporated herein by reference. The impact of these regulatory events on overall MAOA protein function and clinical relevance is described below.

Search for the Functional Polymorphism(s)

Previous in vitro studies have associated the 4-repeat pVNTR with higher levels of transcription than the 3-repeat (Sabol, S., et al. (1998) Hum. Genet., 103, 273-279) incorporated herein by reference. Accurate inference of the haplotypes enabled us to relate AEI ratios directly to specific alleles of the pVNTR and all SNPs in female samples heterozygous for the marker SNPs. In most samples, the main wild-type marker allele was linked to the 4-repeat, which was associated with higher mRNA expression than the 3-repeat in a majority of samples. This result is consistent with previously in vitro data that the 4-repeat pVNTR causes higher expression than the 3-repeat. However, these in vitro results do not assure that the pVNTR has the same influence in human brain tissues. Moreover, the pVNTR is in strong linkage disequilibrium over a large region of the MAOA gene locus, raising the question which domain contains the functional polymorphism. The pVNTR cannot account for all of our observed allelic expression ratios, because the presence of marked AEI in 3 tissues homozygous for the pVNTR resulted in considerably stronger association of the AEI phenotype to the 3′ region containing both marker SNPs, including several completely linked SNPs in the same haplotype block (FIG. 10). However, this analysis is confounded by allelic methylation differences. Because we were unable thus far to measure allele-specific CpG methylation in samples homozygous for the pVNTR, the contribution of methylation in these samples could not be evaluated in this study. Nevertheless, the results favor the presence of a functional polymorphism in the marker SNP region. On the other hand, the data are also consistent with the presence of more than one functional polymorphism, including the pVNTR.

Because of the strong association of AEI with the haplotype block containing the marker SNPs, we tested the two marker SNPs and one SNP in the 3′-UTR (rs1801291, rs6323, and rs3027407), all located in the cDNA, in cell culture. In this approach, one co-transfects equal amounts of wild-type and variant cDNA in an expression vector, followed by AEI analysis of the plasmid DNA and respective mRNA at different time points. The transfection conditions effectively remove epigenetic factors from playing a role. Lack of any detectable AEI for the 3 SNPs, either tested alone or linked together in the same vector, demonstrated that that none of the tested SNPs were functional when analyzed in the context of intronless cDNA constructs. While this excludes a mechanism involving mature mRNA processing and turnover, as we have observed for OPRM1 and MDR1, we cannot exclude possible effects occurring at the level premature hnRNA (maturation and splicing). Moreover, there are other highly linked intronic SNPs in the haplotype block that could be contributing to AEI but cannot be studied with this cDNA approach.

The results of this study have implications for future clinical genetic association studies, providing evidence for the presence of a genetic factors affecting mRNA expression in human brain tissues, which is further modulated by CpG island methylation in female subjects. The use of the pVNTR alone in clinical studies may not accurately represent the true genetic variability in a subject cohort. The finding that promoter methylation affects allelic MAOA transcription and varies considerably between females indicates that epigenetic factors also play a significant role in modulating biogenic amine tone in the CNS of female subjects, and hence mental activity and disorders. However, we have detected no methylation in male brain tissues. This study is the first to use AEI analysis for dissecting genetic and epigenetic factors in human brain tissue. Example 4 will address the biological and clinical significance of these findings.

Materials and Methods

Description of the DNA, and mRNA, and Tissue Samples

Postmortem brain tissue, mRNA and DNA was donated by The Stanley Medical Research Institute's brain collection. We obtained genomic DNA and total mRNA extracted from the prefrontal cortex of 105 individuals previously diagnosed with bipolar disorder (35) or depression (35), and 35 controls. Extracted RNA is from Brodmann's area 46 (dorsolateral prefrontal cortex). Additional brain tissue from 4 of the individuals analyzed above was obtained from the following regions: cerebellum, parietal lobe and occipital lobe. Average post-mortem interval for these samples was 32.9±16.0 hours. Additional demographic data available for these samples included age, sex, cause of death and history of smoking, alcohol use and lifetime use of antipsychotic medication.

DNA Genotyping Using GC Clamp and Differential Melting Curve Analysis

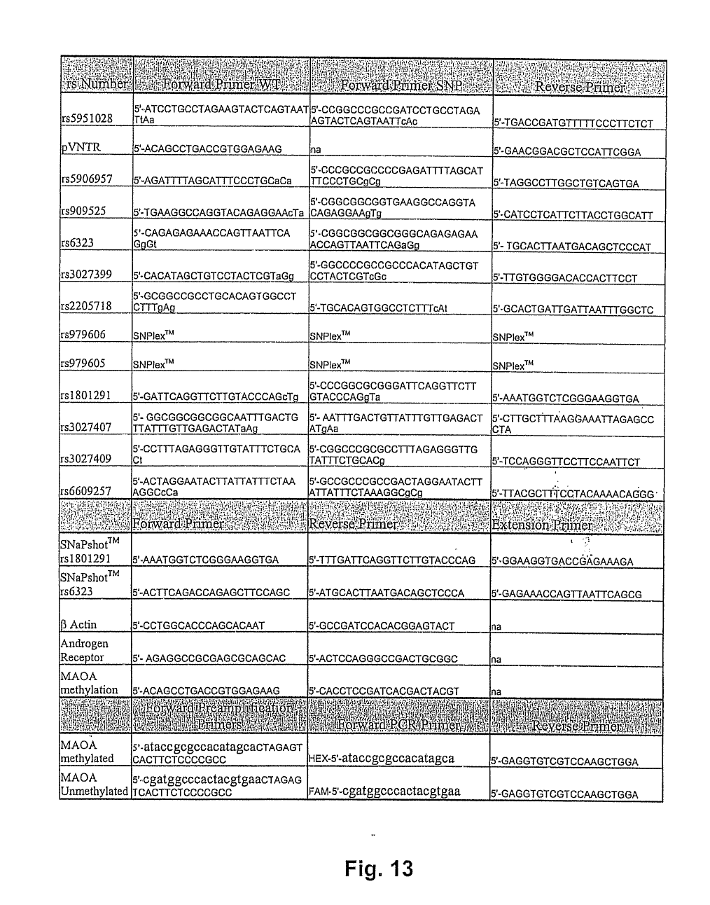

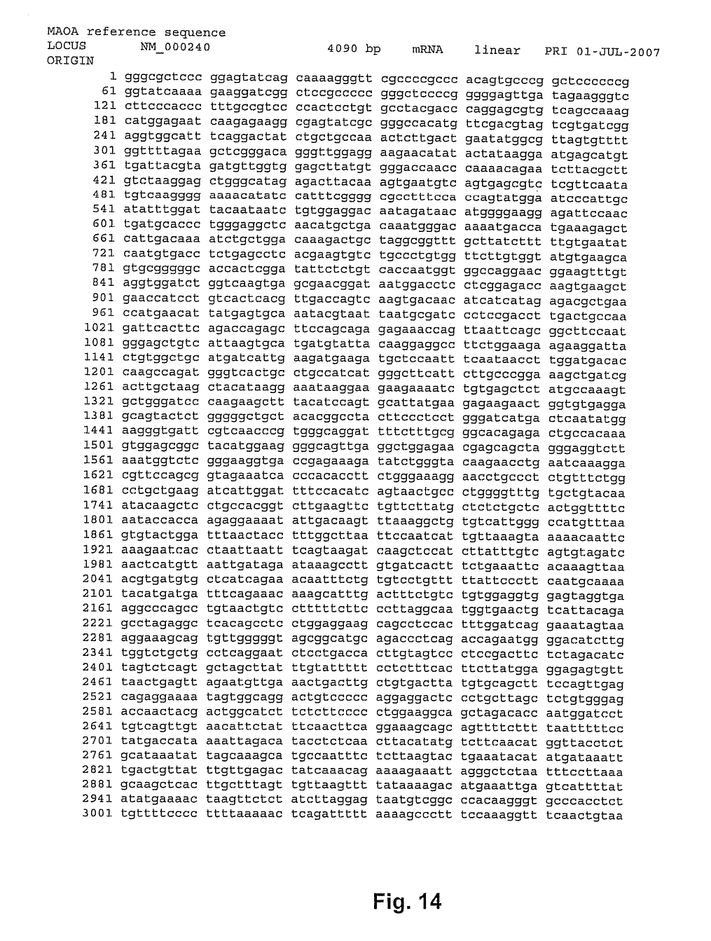

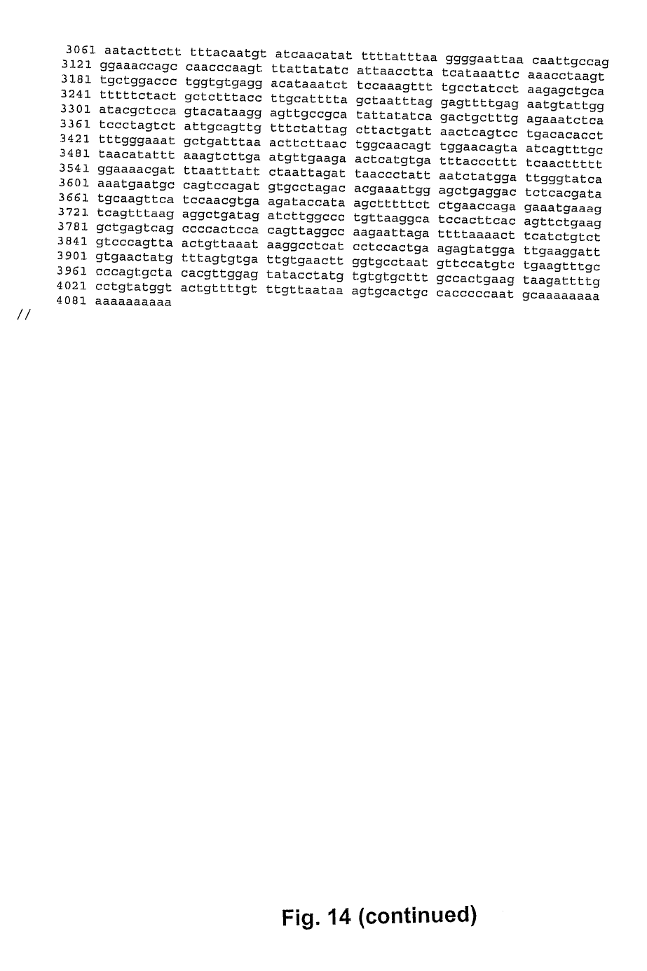

A total of 13 polymorphisms were genotyped spanning the MAOA gene (FIGS. 1 and 13). FIG. 14 contains the mRNA sequence for the MAOA gene. Ten SNPs were genotyped by allele-specific PCR, with primer for one allele containing a GC-rich sequence at the 5′ end. Allele discrimination was achieved with melting curve analysis. Primers for each SNP are listed in FIG. 13.

DNA Genotyping of the Variable Nucleotide Tandem Repeat (pVNTR)

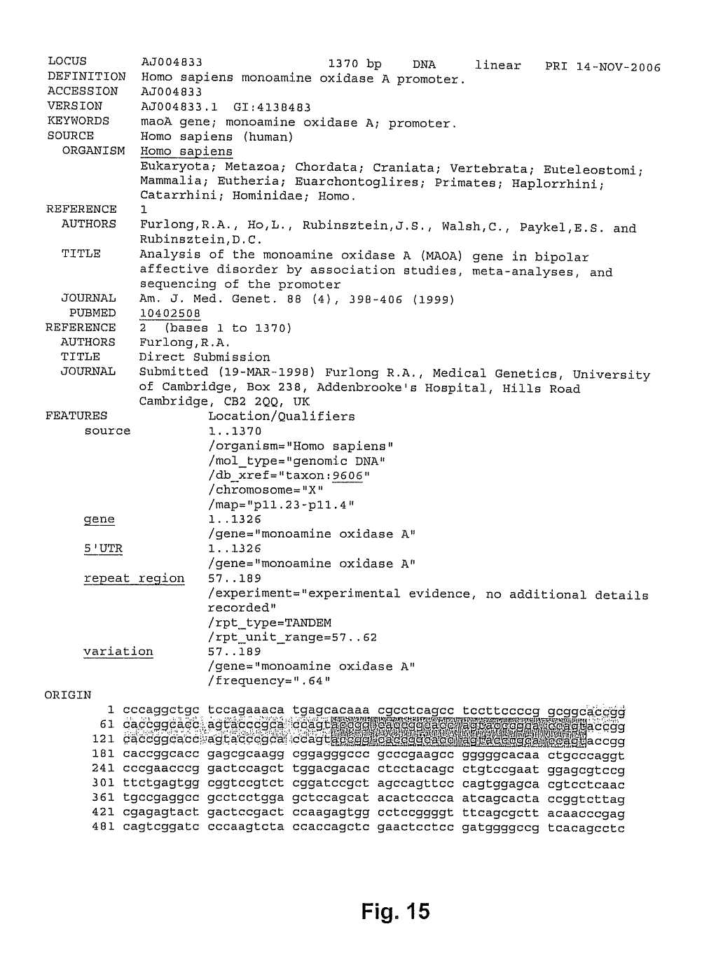

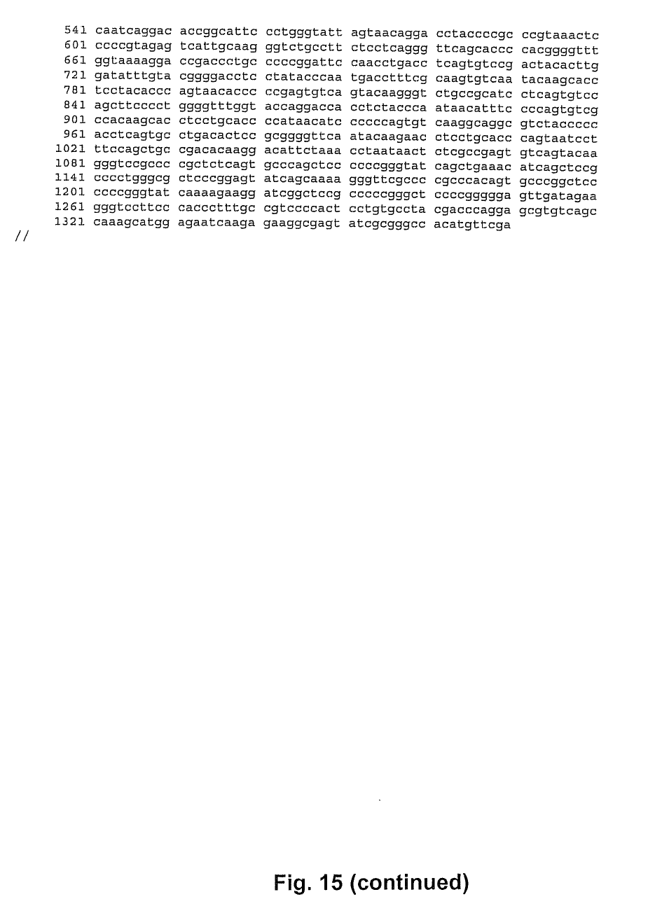

PCR amplification of the promoter VNTR followed a protocol from Sabol et al., with modifications. FIG. 15 contains the sequence for the 4-repeat pVNTR. Primers are listed in FIG. 13. The forward primer was labeled with a fluorescent dye for analysis of the PCR product on an Applied Biosystems 3730 sequence sequencer, separating 3-, 4-, and 5-repeats (3.5-repeats were not encountered and the 5-repeat allele was found in 2 male subjects and thus not analyzed for AEI). PCR cycling conditions were as follows: 1 minute at 95° C., 1 minute at 62° C., 1 minute at 72° C. for 35 cycles.

Haplotype Analysis

105 samples were genotyped. 69 male samples (containing only one MAOA allele) allowed unambiguous assignments of haplotypes. For the female subjects, haplotypes and their frequencies were assigned by an estimation maximization algorithm (HelixTree™ Golden Helix software package. Together with haplotype information from males, this provided unambiguous assignments in essentially all female cases. Importantly, haplotype assignments in female enabled the linkage of the pVNTR (3-repeat and 4-repeat) with the two alleles of the marker SNPs.

cDNA Synthesis

cDNA was synthesized from 105 prefrontal cortex mRNA samples. Approximately 1 μg total RNA was digested with 2 units of DNase 1 in appropriate buffer for 20 minutes at 37° C. Enzyme was inactivated with DNA Free slurry (Ambion). RNA was transferred to new tubes containing 1 μl 10 mM dNTP, 1 μl 0.5 mg/ml Oligo dT and 0.5 μl 2 μM gene specific primers (MAOA SNaPshot and β actin (FIG. 13)). In addition to oligo dT, gene-specific primers for cDNA synthesis targeting a region just downstream of the marker SNP; this was found to enhance significantly the cDNA yield for the region of interest as fragmentation of mRNA renders oligo dT priming less effective. Incubation was at 65° C. for 5 minutes. 4 μl 5× first strand synthesis buffer (Invitrogen), 4 μl RNase-free water and 1 μl RNase inhibitor were added to each reaction and incubated at 42° C. for 2 minutes. 1 ml SuperScript™ (Invitrogen) was added to reaction and incubated at 42° C. for 50 minutes.

Determination of Allelic Ratios of DNA and mRNA Using a Marker SNP

Allele-specific mRNA analysis was performed after PCR amplification of DNA and cDNA, using a primer extension assay based on SNaPshot™ (Applied Biosystems), as described in Pinsonneault, J. et al. (2004) Genetic variants of the human H+/dipeptide transporter PEPT2: analysis of haplotype functions. J. Pharmacol. Exp. Ther., 311, 1088-1096; incorporated herein by reference. The marker SNPs we employed were a synonymous C/T SNP in exon 14 (rs1801291) and a synonymous T/G SNP in exon 8 (rs6323). Use of two marker SNPs, yielded two independent assay procedures for comparison and validation. Primers used are listed in FIG. 13. For each sample, DNA ratios were measured in duplicate, and the mean (±SD) calculated across all samples, assuming that the allelic ratio is unity in female subjects. No single sample deviated by more than 3 standard deviations from the mean. Messenger RNA allelic ratios were measured at least three times for each sample, enabling an assessment for each individual sample, whether cDNA ratios deviated from DNA ratios. We also performed a standard curve experiment for marker SNP rs1801291, using plasmid DNA at the following mixtures: 30, 40, 50, 60, and 70% of one allele relative to the other, at 3 dilutions (1:0, 1:2 and 1:4). R² for the 3 dilutions against the standard ranged from 0.98 to 0.97.

Linkage Disequilibrium Analysis

Pair-wise linkage disequilibrium (LD) was determined for each combination of SNP pairs using HelixTree™ software (Golden Helix, Inc., Bozeman, Mont.). D Prime: an alternative measure of linkage disequilibrium, Lambert, C. (2004) HelixTree™ Genetics Analysis Software. 3.0.6 ed. Golden Helix, Inc., Bozeman, Mont., is calculated by the HelixTree™ software and included in FIG. 2. Because there is no mechanism in the HelixTree™ software to recognize hemizygous genotypes, to accurately determine allele frequencies, correct LD and haplotype frequencies, male genotype data was treated as homozygous (instead of hemizygous), while female genotype data was doubled (to account for doubling of male data).

Statistical Analyses

Alleles from females heterozygous for the indicator SNP were sorted into two groups based on whether they were on the high or low expressing allele. HelixTree™ software was used to test the significance of each genotype to its presence on the high or low expressing allele. Expression (high or low) was used as the dependent variable in a tree analysis, and a 2-loci p-value plot was created for every possible combination of SNPs. The single locus associations included in the analysis were taken for the plot (FIG. 10). The recursive partitioning function of the HelixTree™ software package was used to test for significant associations of AEI results with patient demographics. Student T tests were performed to assay for significant differences in AEI, X-inactivation, and MAOA locus methylation ratios, as well as total MAOA locus methylation and MAOA mRNA expression levels when samples were sorted by disease profile or case versus control.

Plasmid Construction

The MAOA cDNA clone in the vector pCMV6-XL4 was obtained from Origene®, containing the major allele at all 3 SNP positions. We constructed 4 variants by site-directed mutagenesis, containing each SNP variant singly or in combination of all of them. Overlapping primers containing the SNP allele were designed for each SNP (rs6323, rs1801291 and rs3027407) (Table 2).

TABLE 2

| | |
|---|---|
| rs6323 Mut F | GAGAGAAACCAGTTAATTCAGCGGCTTCCAATGGGAGCTG |
| rs6323 Mut R | CAGCTCCCATTGGAAGCCGCTGAATTAACTGGTTTCTCTC |
| rs1801291 Mut F | CCGAGAAAGATATCTGGGTACAAGAACCTGAATCAAAGGACG |
| rs1801291 Mut R | CGTCCTTTGATTCAGGTTCTTGTACCCAGATATCTTTCTCGGA |
| rs3027407 Mut F | GACTGTTATTTGTTGAGACTATCAAACAGAAAAGAAATTAGGGC |
| rs3027407 Mut R | GCCCTAATTTCTTTTCTGTTTGATAGTCTCAACAAATAACAGTC |

Stratagene Quick Change® kit was employed to create each SNP. 50 μl PCR reactions containing primers, reaction buffer, dNTP mix, 50 ng of template, and Pfu enzyme were cycled 18 times with the following parameters: 95° C. for 50 seconds, 60° C. for 50 seconds, 68° C. for 9 minutes. The reaction was treated with Dpn I (20 units) at 37° C. for 1 hour. Reactions were transformed into competent XL10 Gold (Stratagene). Plasmids were purified from colonies and sequenced to identify site-directed mutations.

Cell Culture and Transient Transfection

CHO-K1 cells were cultured in F-12 nutrient medium (Invitrogen) supplement with 10% fetal bovine serum, 100 units/ml penicillin, and 100 μg/ml streptomycin at 37° C. with 5% CO₂. Twenty-four hours before transfection, cells were seeded into 6 or 12-well dishes. Transfections were performed using Lipofectamine 2000 reagent (Invitrogen) according to the manufacturer's protocol, using equal amounts of the plasmid carrying the wild type cDNA and of the variant cDNA. Cells were collected 8, 24 and 48 h after treatment. For plasmid DNA preparation, cells were trypsinized and collected. Plasmid DNA was prepared using a Qiagen DNA miniprep kit. For RNA preparation, cells were lysed with TRIzol reagent and prepared as described above. MAOA plasmid and mRNA were analyzed by SNaPshot after conversion to cDNA. Control assays without transfection yielded no detectable amounts of hamster genomic DNA and mRNA.

X Chromosome Inactivation Assay

The method employed to measure the X chromosome inactivation ratio was modified from the procedure described in Sandovici, I., et al. (2004) A longitudinal study of X-inactivation ratio in human females. Hum. Genet., 115, 387-392. Gene specific primers (FIG. 13) were used to amplify a polymorphic region of the androgen receptor gene in genomic DNA. The DNA was either untreated or had previously been digested overnight with Hha I methyl-sensitive restriction endonuclease. The forward primer was labeled with a fluorescent dye so that products could be visualized on an ABI 3730 analyzer. PCR conditions were as follows: 30 cycles of 94° C. for 1 minute, 68° C. for 1 minute and 72° C. for 1 minute. Peak areas for both amplification products from each sample were determined, and ratios were calculated. Each sample was assayed 3 times.

MAOA Methylation Assay I

The method used to measure differential methylation at the MAOA locus was modified from an X inactivation protocol by Hendricks et al. (Hum. Mol. Genet., 1, 187-94 (1992); incorporated herein by reference), but instead of amplifying a polymorphic dinucleotide region in intron 1, the pVNTR was amplified. Genomic DNA was digested overnight at 30° C. with 5 units of Sma I in appropriate buffer. The forward, fluorescently labeled primer used to genotype the pVNTR (FIG. 13) was combined with a reverse primer (FIG. 13) just down stream of a Sma I site shown to be methylated Hendricks et al. (Hum. Mol. Genet., 1, 187-94 (1992); incorporated herein by reference). PCR conditions were as follows: minute at 95° C., 1 minute at 62° C., 2 minutes at 72° C. for 35 cycles. 8 ml of each PCR reaction was digested with 10 units of Sst I enzyme overnight at 37° C., in appropriate buffer, to shorten the PCR product so that it would be visible by capillary electrophoresis.

MAOA Methylation Assay II

This method quantitates both total and allelic MAOA methylation. First, is divided into two equal samples, each consisting of 20-120 ng of DNA in 1×HhaI digestion buffer. One sample is digested with 10 units of the methylation sensitive restriction enzyme (HhaI). Digested and undigested DNA were tagged with two different address primers in a quantitative pre-amplification step, using a common 3′ MAOA reverse primer (down stream of the Hha I site), and two distinct forward primers. Each forward primer targets the same MAOA sequence located upstream of the pVNTR, but is tagged with a different address at its 5′ end. Specific, quantitative pre-amplification was achieved using 5 nM primer concentrations for 8 cycles of denaturation at 95° C. for 30 seconds, then annealing/extension at 60° C. for 2 hours. After separate PCR pre-amplifications, equal volumes of the two samples are mixed. One microliter of the combined pre-amplified DNA is then competitively amplified for 25 PCR cycles using 300 nM concentrations of the common 3′-primer and FAM or HEX fluorescently labeled 5′ primers targeting the tag sequence in the forward primer (representing cut and uncut DNA). Using the ABI 3730 instrument a portion of the resulting PCR product was analyzed by capillary electrophoresis. One obtains two fluorescent peaks which are proportional to the amount of amplifiable HhaI-pretreated and untreated DNA. The ratio of the peaks is used to calculate the % methylation. For samples heterozygous for the pVNTR, two sets of peaks were obtained representing cut and uncut DNA from the 3- and 4-repeat alleles (in female carriers), while for all other samples (including males) we determine the level of overall methylation in %.

To achieve equal PCR amplification efficiency for both alleles, we tested several combinations of address primers to arrive at the optimal pair for MAOA. To construct standard curves, pooled uncut DNA was divided into portions and carried through the entire procedure without Hha I digestion of any one sample. FAM and HEX peak areas were obtained from the ABI 3730 and the ratios plotted, yielding a linear standard curve (r>0.99).

Quantitative mRNA Analysis by RT-PCR

PCR was performed on cDNA samples using SYBR green dye on an ABI 7000 sequence detection system (Applied Biosystems, Foster City, Calif., USA). PCR (21 μL) was performed in standard 96-well plates with heat-activated Taq DNA polymerase and SYBR Green. SYBR green fluorescence was measured after each cycle. After full amplification, the fluorescence intensity of the PCR product was measured from 60° C. to 92° C. at a temperature gradient of 0.2° C./min to control for spurious amplification with different melting curves. Forward and reverse primers listed in FIG. 13 were used to amplify MAOA and β-actin transcripts. Each reaction was replicated once. Cycle thresholds (Ct), at which an increase in reporter fluorescence above a baseline signal is detected, were determined with ABI 7000 SDS software. Replicate cycle thresholds were averaged, and MAOA expression levels in arbitrary units were calculated by subtracting the β-actin Ct from the MAOA Ct to get a ΔCt. Arbitrary units for each sample=100/2^{Δct}.

### Example 2

### Tryptophan Hydroxylase 2 (TPH2) Functional Polymorphisms

Tryptophan hydroxylase isoform 2 (TPH2) is expressed in serotonergic neurons in the raphe nuclei, where it catalyzes the rate-limiting step in the synthesis of the neurotransmitter serotonin. In search for functional polymorphisms within the TPH2 gene locus, we measured allele-specific expression of TPH2 mRNA in sections of human pons containing the dorsal and median raphe nuclei. Differences in allelic mRNA expression—referred to as allelic expression imbalance (AEI)—are a measure of cis-acting regulation of gene expression and mRNA processing. Two marker SNPs, located in exons 7 and 9 of TPH2 (rs7305115 and rs4290270, respectively), served for quantitative allelic mRNA measurements in pons RNA samples from 27 individuals heterozygous for one or both SNPs. Significant AEI (ranging from 1.2 to 2.5-fold) was detected in 19 out of the 27 samples, implying the presence of cis-acting polymorphisms that differentially affect TPH2 mRNA levels in pons. For individuals heterozygous for both marker SNPs, the results correlated well (r=0.93), validating the AEI analysis. AEI is tightly associated with the exon 7 marker SNP, in 17 of 18 subjects. Remarkably, expression from the minor allele exceeded that of the major allele in each case, possibly representing a gain-of-function. Genotyping of twenty additional TPH2 SNPs identified a haplotype block of five tightly linked SNPs for which heterozygosity is highly correlated with AEI and overall expression of TPH2 mRNA. These results reveal the presence of a functional cis-acting polymorphism, with high frequency in normal human subjects, resulting in increased TPH2 expression levels. The SNPs that correlate with AEI are closely linked to TPH2 SNPs previously shown to associate with major depression and suicide.

Introduction

Tryptophan hydroxylase (TPH) catalyzes the rate-limiting step in the synthesis of serotonin (5-hydroxytryptamine; 5-HT), a neurotransmitter that plays an important role in the regulation of mood. Dysregulation of serotonergic activity has been associated with a number of mental disorders including major depression, anxiety disorders and suicidal behavior. Most antidepressant drugs, including the serotonin-selective reuptake inhibitors (SSRIs) and many tricyclic antidepressants (TCAs), increase levels of extracellular serotonin by inhibiting its reuptake or blocking its metabolism. Tryptophan hydroxylase 2 (TPH2) is a recently discovered isoform of TPH that is specifically expressed in the brain, with particularly high expression in the serotonergic neurons of the raphe nuclei. The dorsal and media raphe nuclei are the major source of serotonin in the forebrain, including areas implicated in mood and anxiety disorders.

Because TPH2 is strategically placed to regulate serotonin levels in the brain, there is currently great interest in identifying genetic variants that affect the level of TPH2 enzymatic activity or control the levels of expression of the TPH2 gene. Extensive DNA sequencing of the TPH2 gene has revealed that polymorphisms that change the amino acid sequence of the TPH2 protein are rare. The focus of research has therefore now changed to identifying genetic variants that influence the TPH2 gene expression.

Recently, measurement of mRNA allelic expression imbalance (AEI) has emerged as a powerful method for identifying genetic variants that influence the expression of mRNAs (Yan, H, et al. (2002) Science, 297, 1143; Bray, N J, et al. (2003) Hum Genet, 113, 149-153) each incorporated herein by reference. In this method, relative levels of mRNA expressed from each of two alleles are measured using RNA isolated from individuals who are heterozygous for a marker single nucleotide polymorphism (SNP) within the mRNA. Using this method, it is possible to reliably detect differences in expression levels between alleles as small as 20%. Because comparisons between expression levels are made using single samples of RNA isolated from specific organs or tissues, variation between individuals that arise from differences in environmental factors, physiological states, or trans-acting factors are minimized: the mRNA from each allele acts as the control for the other. This technique has been used to quantify AEI of mRNAs encoding human: H⁺/dipeptide transporter 2 (PEPT2) (Pinsonneault, J, et al. (2004) J Pharmacol Exp Ther, 311, 1088-1096); p-glycoprotein (MDR1) (Wang, D, et al. (2005) Pharmacogenet Genomics, 15, 693-704); the μ-opiate receptor (OPRM) (Zhang, Y, et al. (2005) J Biol Chem, 280, 32618-32624), and the serotonin transporter (SERT) (Lim, J E, et al. (2006) Mol Psychiatry, July; 11 (7):649-62. Epub 2006 Jan. 24) each incorporated herein by reference.

The goal of this study was to determine whether allele-specific mRNA expression of TPH2 gene occurs and, if so, identify cis-acting genetic elements that predict high or low levels of expression.

Materials and Methods

Materials—Frozen sections of rostral pons containing the dorsal and median raphe nuclei from 48 individuals were purchased from the Brain and Tissue Bank for Developmental Disorders (University of Maryland, Baltimore). The demographics of this collection have been described in Lim, J E, et al. (2006) Mol Psychiatry, July; 11 (7):649-62. Epub 2006 Jan. 24), incorporated herein by reference. Oligonucleotide primers were designed using the program Oligo 4.0 (National Biosciences Inc., Plymouth, Minn.) and synthesized by Integrated DNA Technologies (Coralville, Iowa).

Isolation of DNA and RNA from human pons—Isolation of DNA and RNA from the tissue samples in our collection has been described in Lim, J E, et al. (supra). Briefly, frozen sections of pons were incubated in 10 volumes of RNAlater-ICE Frozen Tissue Transition solution (Ambion Inc, Austin, Tex.) overnight at −80° C. to maximize recovery of DNA and RNA. The next day, a small piece of tissue from the ventral edge of each sample was removed and homogenized in DNA lysis buffer for isolation of genomic DNA and the remaining portion of the sample homogenized in Trizol reagent (Invitrogen) for isolation of total RNA.

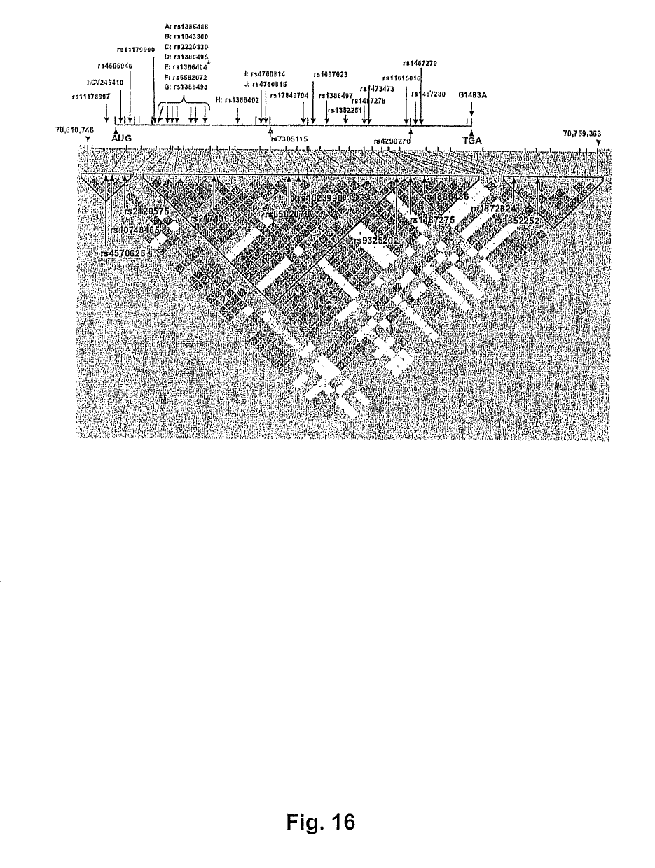

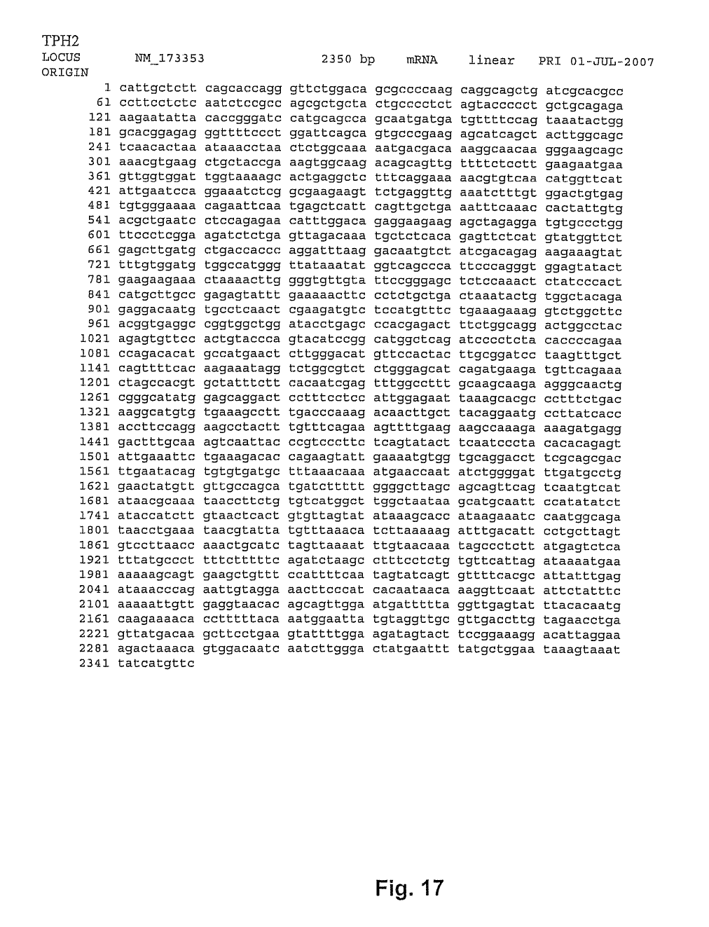

Genotyping—Genotyping of TPH2 SNPs using SNaPshot primer extension assays was carried out as previously described in Lim, J E, et al. (supra). Briefly, short (100-300 bp) segments of genomic DNA were PCR-amplified using pairs of synthetic oligonucleotide primers that flank each SNP. Following amplification, the unincorporated dNTPs were inactivated with antarctic alkaline phosphatase (New England Biolabs) and excess primers degraded with exonuclease I (New England Biolabs). The PCR products were used as templates in SNaPshot primer extension assays (Applied Biosystems, Foster, Calif., USA), using extension primers designed to anneal to the amplified DNA immediately adjacent to the SNP site. The resulting fluorescently-labeled primers were analyzed by capillary electrophoresis using an ABI3730 DNA analysis system and Gene Mapper 3.0 software (Applied Biosystems, Inc.). The TPH2 SNPs we examined are listed in Table 3. The locations of these SNPs within the TPH2 gene are shown in FIG. 16. Sequences of the PCR amplification and primer extension primers and reaction conditions for each primer set used for genotyping are shown in Table 4. FIG. 17 contains the mRNA sequence for TPH2 gene.

TABLE 3
TPH2 SNPs examined in this study

| | | Location on | Location | | |
|---|---|---|---|---|---|
| | | chromosome | within | Allele | |
| SNP # | dbSNP# | 12 | TPH2 gene | frequencies | Hetero-zygosity |
| 01 | rs4570625 | 70618190 | upstream | G/T = 0.72/0.28 | 0.403 |
| 02 | rs10748185 | 70622122 | intron 2 | A/G = | 0.500 |
| | | | | 0.51/0.49 | |
| 03 | rs2129575 | 70626340 | intron 4 | G/T = 0.74/0.26 | 0.385 |
| 04 | rs1386488 | 70630885 | intron 5 | A/C = | 0.255 |
| | | | | 0.85/0.15 | |
| 05 | rs1843809 | 70634965 | intron 5 | T/G = 0.83/0.17 | 0.282 |
| 06 | rs1386495 | 70638589 | intron 5 | T/C = 0.83/0.17 | 0.282 |
| 07 | rs1386494 | 70638810 | intron 5 | G/A = | 0.211 |
| | | | | 0.88/0.12 | |
| 08 | rs6582072 | 70640744 | intron 5 | G/A = | 0.282 |
| | | | | 0.83/0.17 | |
| 09 | rs2171363 | 70646531 | intron 5 | C/T = 0.53/0.47 | 0.498 |
| 10 | rs4760815 | 70658496 | intron 6 | T/A = 0.65/0.35 | 0.455 |
| 11 | rs7305115 | 70659129 | exon 7 | G/A = | 0.455 |
| | | | | 0.65/0.35 | |
| 12 | rs6582078 | 70661158 | intron 7 | T/G = 0.60/0.40 | 0.480 |
| 13 | rs1023990 | 70668514 | intron 7 | T/C = 0.79/0.21 | 0.332 |
| 14 | rs1007023 | 70674641 | intron 8 | T/G = 0.89/0.11 | 0.196 |
| 15 | rs1352251 | 70684161 | intron 8 | T/C = 0.59/0.41 | 0.484 |
| 16 | rs1473473 | 70690645 | intron 8 | G/A = | 0.211 |
| | | | | 0.88/0.12 | |
| 17 | rs9325202 | 70693744 | intron 8 | G/A = | 0.455 |
| | | | | 0.65/0.35 | |
| 18 | rs1487275 | 70696559 | intron 8 | T/G = 0.78/0.22 | 0.343 |
| 19 | rs1386486 | 70698487 | intron 8 | C/T = 0.61/0.39 | 0.476 |
| 20 | rs4290270 | 70702502 | exon 9 | A/T = 0.63/0.37 | 0.466 |
| 21 | rs1872824 | 70716581 | intron 9 | C/T = 0.64/0.36 | 0.461 |
| 22 | rs1352252 | 70738308 | downstream | A/G = | 0.493 |
| | | | | 0.56/0.44 | |

TABLE 4
Primers for allelic expression imbalance
measurement (all shown in 5′ to 3′)

| Marker | Forward | Reverse | Extension |
|---|---|---|---|
| SNP(s) | PCR primer | PCR primer | primer |
| rs7305115 | 5′ACGAGACTTTC | 5′TTAATTCTCCAA | 5′GATCCCCTCTA |
| | TGGCAGGACTG3′ | TGGAGGAAAGGA3′ | CACCCC3′ |
| rs4290270 | 5′ACGAGACTTTC | 5′TTAATTCTCCAA | 5′AAAGGAGTCCT |
| | TGGCAGGACTG3′ | TGGAGGAAAGGA3′ | GCTCCATA3′ |

Linkage disequilibrium (LD) and haplotype analysis—D′ values for each pair of SNPs and estimated haplotype frequencies were calculated using Haploview (version 3.3; available online at www.broad.mit.edu/mpg/haploview/; Barrett, J C, et al. (2005) Bioinformatics, 21, 263-265) incorporated herein by reference, Predicted diplotypes for each individual in our collection were calculated from the genotyping data using HelixTree™ (GoldenHelix, Inc., Bozeman, Mont.).

Allelic Expression Imbalance (AEI) measurements—Measurements of allele-specific mRNA expression were carried out as described previously in Lim, J E, et al. (supra). Briefly, RNA from each sample was treated with RNase-Free DNase Set (Qiagen) for 15 min and re-isolated using QIAGEN RNeasy columns. Complementary DNA (cDNA) was generated from 1 μg RNA in 20 μl reaction mixes containing 1 μl (200 U) Superscript II reverse transcriptase (Invitrogen, Carlsbad, Calif.), 1 μl of 1 μM oligo(dT)₂₀ primer (Invitrogen), 1 μl of 10 mM dNTP mix (Invitrogen), 0.5 μl of 1 μM TPH2 gene-specific primer (5′-TTAATTCTCCAATGGAGGAAAGGA-3′), 4 μl of 5× first-strand buffer (Invitrogen), 1 μl of RNaseOUT (40 units/μl), and RNase-free water. A cDNA segment containing marker SNPs rs7305115 and rs4290270 was amplified using Taq DNA polymerase (Promega), the forward primer 5′-ACGAGACTTTCTGGCAGGACTG-3′, and the reverse primer 5′-TTAATTCTCCAATGGAGG-AAAGGA-3′ with the following cycles: [1× (5 min at 95° C.); 35× (30 sec at 95° C., 30 sec at 60° C., 1 min at 72° C.) 1× (7 min at 72° C.)]. Following amplification, the unincorporated dNTPs were inactivated with antarctic alkaline phosphatase (New England Biolabs) and excess primers degraded with exonuclease I (New England Biolabs). SNaPshot Primer extension assays were carried out using the extension primer 5′-GATCCCCTCTACACCCC-3′ for rs7305115 and 5′-AAAGGAGTCCTGCTCCATA-3′ for rs4290270 with the following cycles: [25× (10 sec at 96° C., 5 sec at 50° C., 30 sec at 72° C.)]. Unincorporated fluorescent dNTP analogs were removed by incubation with 1.0 unit of intestinal calf phosphatase (10.00 U/ml; New England Biolabs) for 3 h at 37 C. The primer extension products were resolved by capillary electrophoresis using an Applied Biosystems 3730 DNA Analyzer and quantified using the Gene Mapper™ 3.0 software (Applied Biosystems).

Addition of different fluorescently labeled dideoxynucleotides onto the 3′-end of the primers produces oligonucleotides with slightly different electrophoretic mobilities and distinct fluorescence spectra. Because different fluorophores differentially affect the efficiency of nucleotide incorporation and have different fluorescence yields, peak area ratios of genomic DNA diverge from the theoretical ratio of 1.0. The measured ratios for genomic DNA were therefore normalized to 1.0 by multiplying each measured ratio by the inverse of the mean of the genomic DNA ratios [correction factor=1/(mean of measured genomic DNA ratios)]. Two tissue samples #1230 and #1609) yielded allelic DNA ratios significantly different from the mean (>4 standard deviations, indicating the presence of a gene dosage effect), and were excluded from the calculated mean DNA ratios. RNA (i.e., cDNA) ratios from heterozygous samples were multiplied by the same correction factor. SNaPshot assays were performed 3× with genomic DNA and 3× with three independent cDNA preparations per sample.

Real-time PCR—TPH2 and glyceraldehydes-3-phosphate dehydrogenase (GAPDH) mRNA levels were measured by real-time PCR using an ABI 7000 DNA sequence detection system (Applied Biosystems, Foster City, Calif.) as previously described in Lim, J E, et al. (supra). Briefly, TPH2 or GAPDH complementary DNA (cDNA) was synthesized from 1 μg total pons RNA using reverse-transcriptase and the primers: 5′-TTAATTCTCCAATGGAGGAAAGGA-3′ (TPH2) or 5′-GTGTGGTGGGGGACTGAGTGTG-3′ (GAPDH). Segments of TPH2 or GAPDH cDNAs were amplified using TPH2- or GAPDH-specific primer sets and heat-activated Taq DNA polymerase in reaction mixes containing dNTPs, buffer, SYBR-Green and a reference dye (Applied Biosystems, Foster City, Calif.). The TPH2 amplification primers were: 5′-ACGAGACTTTCTGGCAGGACTG-3′ (forward) and 5′-TTAATTCTCCAATGGAGGAAAGGA-3′ (reverse) and the GAPDH amplification primers were: 5′-CAGCAAGAGCACAAGAGGAAGAGAGA-3′ (forward) and 5′-GTGTGGTGGGGGACT-GAGTGTG-3′(reverse). Amplification conditions consisted of a 10-min preincubation at 95° C. to activate the Taq DNA polymerase, followed by 40 cycles of denaturation at 95° C. for 15 sec and primer annealing and extension for 1 min at 60° C. PCR product melting curves were examined to confirm the homogeneity of PCR products. TPH2 mRNA measurements were expressed as cycle thresholds (C_{T}) and normalized by subtracting C_{T} values obtained with GAPDH mRNA.

Statistics—Differences between corrected genomic and mRNA (cDNA) ratios were tested for statistical significance using the General Linear Model (GLM) procedure in SAS (SAS Institute Inc., Cary, N.C.). Agreement between AEI measurements using the marker SNP rs7305515 or rs4290270 was assessed by calculating the Pearson correlation coefficient for mean AEI values for individuals heterozygous for both SNPs (n=13). Correlations between heterozygosity of TPH2 SNPs and AEI of THP2 mRNA were examined by calculating Kappa-coefficients using SPSS(SPSS Inc. Chicago, Ill.). Agreement was defined to be either heterozygous and TPH2 AEI>1.2, or homozygous with TPH2 AEI<1.2. Exact two-sided p-values for the significance of the kappa estimate were computed.

Transfection of CHO Cells and Measurement of TPH2 mRNA Levels:

Expression vectors: Reverse transcriptase was used to synthesize cDNA from RNA isolated from an individual homozygous for the TPH2 A-allele of rs7305115. An expression vector encoding the TPH2 A-allele was constructed by subcloning this cDNA in the BamHI/Xba I site of pcDNA3.1. An expression vector encoding the TPH2 G-allele was produced by using site-directed mutagenesis to convert the A-allele to a G. DNA sequencing of the TPH2 coding regions confirmed that the only difference between the expression vectors was the presence of the A- or G-allele.

Transfections: CHO cells were cultured at 37° C. in a humidified incubator at 5% CO2 in Ham's F-12 Medium plus 10% fetal bovine serum, 100 U/ml penicillin and 100 mg/ml streptomycin. The day before transfection, cells were re-plated into 6-well plates at approximately 50% confluency. Transfection of TPH2 expression constructs was performed using lipofectamine 2000 reagent according to the manufacturer's protocol. To determine the time course of TPH2 expression, CHO cells were transfected with 4 mg TPH2-A expression vector. Total RNA was isolated at 5, 8 12, 24 48 72 h after transfection to determine peak levels of TPH2 mRNA expression. For mRNA stability studies, CHO cells were co-transfected with 2 mg (each) of TPH2-A and TPH2-G. Twenty-hour hours after transfection, the cells were treated with vehicle or 10 mg/ml actinomycin D for 0, 1, 2, 5, 8, and 12 hrs. At these time points, cell cultures were either trypsinized and collected for plasmid DNA preparation using QIAGEN mini prep kits, or lysed with 1 ml Trizol, followed by RNA purification with QIAGEN easy RNA mini prep kits. Contaminating DNA in the RNA samples was eliminated by DNase I treatment prior to column purification. The amplification primers did not amplify cDNA prepared from untransfected CHO cells, indicating that the primers used in this study specifically detected TPH2 mRNA produced from the expression vectors.

mRNA quantification: TPH2 mRNA levels were measured in transfected CHO cells by reverse transcription followed by real-time PCR analysis. Endogenous β-actin mRNA was also measured using primers specific for hamster β-actin. The expression of TPH2 was expressed as the ratio of TPH2 mRNA β-actin mRNA. To ensure absence of genomic DNA in RNA samples, control tubes containing the same amounts of RNA without reverse transcriptase were also assayed. Real-time PCR analysis showed the cycle thresholds from these control samples were higher than 30 cycles, similar to blank controls, showing that genomic DNA levels were undetectable.

Results

To identify samples suitable for TPH2 mRNA AEI measurements, we genotyped chromosomal DNA from each of our samples for two marker SNPs: rs7305115 (exon 7) and rs4290270 (exon 9). (See FIG. 16 for the locations of these and additional TPH2 SNPs.) Among the 48 individuals in our collection, 18 were heterozygous for rs7305115 (G/A) and 22 heterozygous for rs4290270 (A/T). Five individuals were heterozygous only for rs7305115 (#1027, 1230, 1540, 1551, 1609), nine individuals were heterozygous only for rs4290270 (#1054, 1104, 1169, 1430, 1442, 1486, 1546, 1613, 1614), and thirteen individuals were heterozygous for both SNPs (#813, 879, 914, 917, 1078, 1101, 1103, 1105, 1112, 1135, 1279, 1489, and 1607). Alleles of both marker SNPs were in Hardy-Weinberg equilibrium within the complete collection of 48 individuals (not shown).

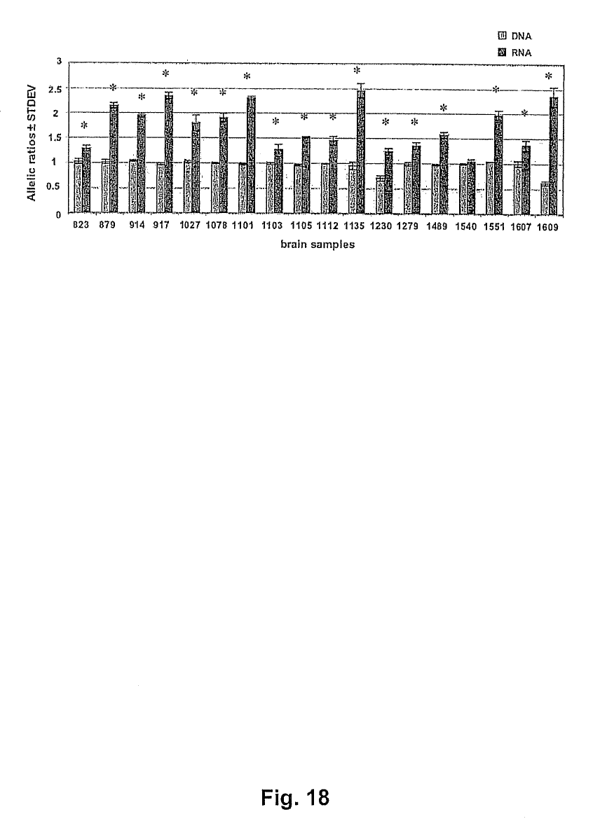

FIG. 18 shows the results of mRNA AEI measurements for the 18 individuals heterozygous for rs7305115. Seventeen of the samples (94%) showed higher expression of mRNA for the A-allele compared to the G-allele, with ratios ranging from 1.2 to 2.5 (Table 5). The G-allele represents the reference sample (wild-type), while the A-allele is a minor, albeit frequent, variant. Sample 1540 showed no significant AEI. All but two of the samples yielded allelic ratios for genomic DNA close to the expected value of 1.0. Two samples (#1230 and #1609) consistently yielded ratios significantly below 1.0. These low ratios suggest a possible duplication in the TPH2 locus containing the G-allele.

TABLE 5
Measurements of AEI using the marker SNPs rs7305115 and rs4290270

| | rs7305115 (A/G) | | | rs4290270 (T/A) | | |
|---|---|---|---|---|---|---|
| | DNA | RNA | | DNA | | |
| | mean ± | mean ± | | mean ± | RNA | |
| Sample # | S.D. | S.D. | p | S.D. | mean ± S.D. | p |
| 813 | 1.03 ± 0.05 | 1.28 ± 0.06 | <0.0001* | 1.04 ± 0.06 | 0.99 ± 0.14 | 0.8467 |
| 879 | 1.02 ± 0.04 | 2.14 ± 0.05 | <0.0001* | 1.13 ± 0.07 | 1.91 ± 0.07 | <0.0001* |
| 914 | 1.04 ± 0.02 | 1.95 ± 0.04 | <0.0001* | 1.01 ± 0.03 | 1.94 ± 0.06 | <0.0001* |
| 917 | 0.98 ± 0.02 | 2.34 ± 0.06 | <0.0001* | 1.02 ± 0.05 | 2.55 ± 0.38 | <0.0001* |
| 1027 | 1.03 ± 0.03 | 1.79 ± 0.16 | <0.0001* | — | — | — |
| 1054 | — | — | — | 0.88 ± 0.06 | 0.97 ± 0.06 | 0.6504 |
| 1078 | 1.00 ± .001 | 1.90 ± 0.08 | <0.0001* | 0.96 ± 0.06 | 1.76 ± 0.05 | <0.0001* |
| 1101 | 0.99 ± 0.02 | 2.30 ± 0.03 | <0.0001* | 0.97 ± 0.02 | 2.41 ± 0.032 | <0.0001* |
| 1103 | 1.00 ± 0.03 | 1.28 ± 0.100 | <0.0001* | 0.97 ± 0.02 | 1.22 ± 0.112 | 0.0029* |
| 1104 | — | — | — | 0.98 ± 0.02 | 1.09 ± 0.04 | 0.212 |
| 1105 | 0.97 ± 0.02 | 1.52 ± 0.02 | <0.0001* | 0.98 ± 0.04 | 1.25 ± 0.05 | 0.0009* |
| 1112 | 1.00 ± 0.01 | 1.50 ± 0.08 | <0.0001* | 0.97 ± 0.03 | 1.22 ± 0.02 | 0.0035* |
| 1135 | 0.95 ± 0.08 | 2.46 ± 0.15 | <0.0001* | 1.04 ± 0.08 | 2.52 ± 0.16 | <0.0001* |
| 1169 | — | — | — | 1.02 ± 0.02 | 1.14 ± 0.05 | 0.0442 |
| 1230 | 0.71 ± 0.05 | 1.23 ± 0.06 | 0.0006* | | | |
| 1279 | 1.00 ± 0.03 | 1.35 ± 0.06 | <0.0001* | 0.95 ± 0.02 | 0.94 ± 0.03 | 0.394 |
| 1430 | — | — | — | 1.06 ± 0.02 | 1.25 ± 0.12 | <0.0001* |
| 1442 | — | — | — | 1.08 ± 0.19 | 0.92 ± 0.11 | 0.251 |
| 1486 | — | — | — | 0.97 ± 0.06 | 0.95 ± 0.03 | 0.5221 |
| 1489 | 0.98 ± 0.01 | 1.57 ± 0.06 | <0.0001* | 0.99 ± 0.07 | 1.62 ± 0.07 | <0.0001* |
| 1540 | 1.00 ± 0.02 | 1.05 ± 0.04 | 0.0943 | — | — | — |
| 1546 | — | — | — | 0.98 ± 0.03 | 1.1 ± 0.08 | 0.1467 |
| 1551 | 1.04 ± 0.00 | 1.97 ± 0.09 | <0.0001* | — | — | — |
| 1607 | 1.00 ± 0.06 | 1.37 ± 0.10 | <0.0001* | 1.11 ± 0.04 | 1.72 ± 0.23 | <0.0001* |
| 1609 | 0.63 ± 0.04 | 2.35 ± 0.20 | <0.0001* | | | |
| 1613 | — | — | — | 0.89 ± 0.06 | 1.06 ± 0.06 | 0.3639 |
| 1614 | — | — | — | 1.0 ± 0.07 | 1.29 ± 0.07 | 0.0002* |

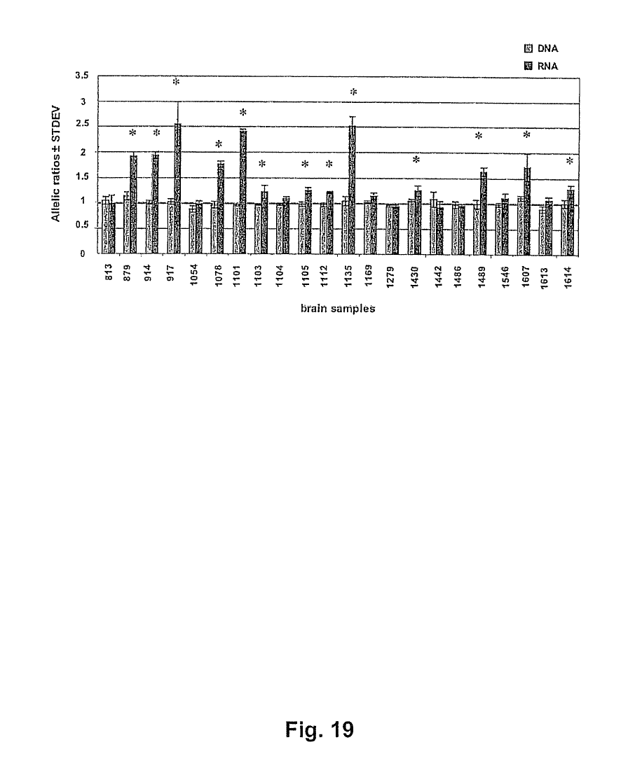

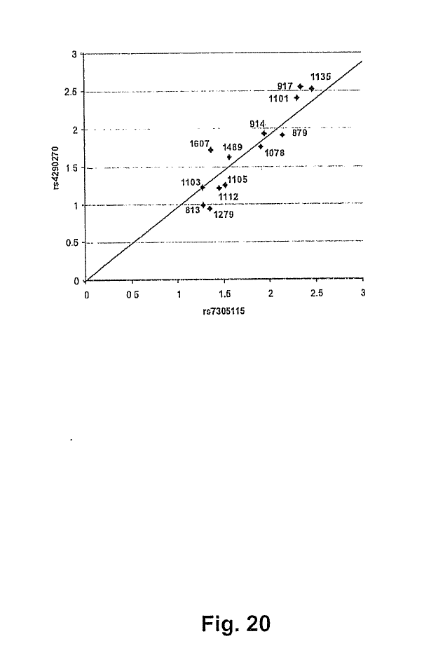

FIG. 19 shows the results of AEI assays for the 22 individuals heterozygous for rs4290270. There was significant AEI in 13 RNA samples, with higher expression of the T-allele (again the frequent minor variant). Ratios ranged from 1.2 to 2.5 (Table 2). Thirteen of the 22 samples were heterozygous for both marker SNPs, affording the opportunity to validate the results obtained with the marker SNP rs7305115. FIG. 20 shows that there is an excellent correlation between AEI measurements made using the two marker SNPs.

The results in FIGS. 18-20) show that heterozygosity of rs7305115 is highly correlated with TPH2 mRNA AEI (17/18=94%), while heterozygosity of rs4290270 is less highly correlated (13/22=59%). These results raise the possibility that rs7305115 is tightly linked to the “functional’ polymorphism that controls levels of TPH2 mRNA expression, or is itself a functional polymorphism.

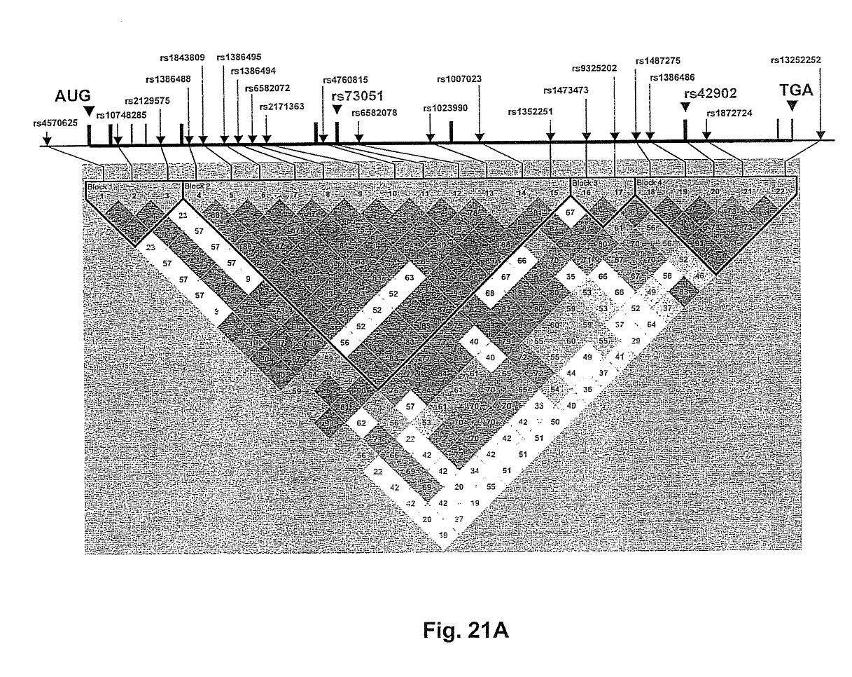

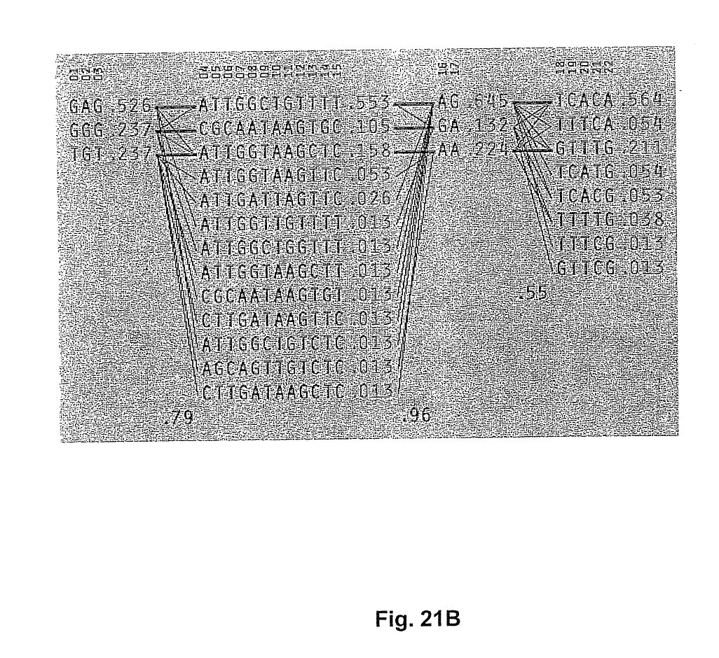

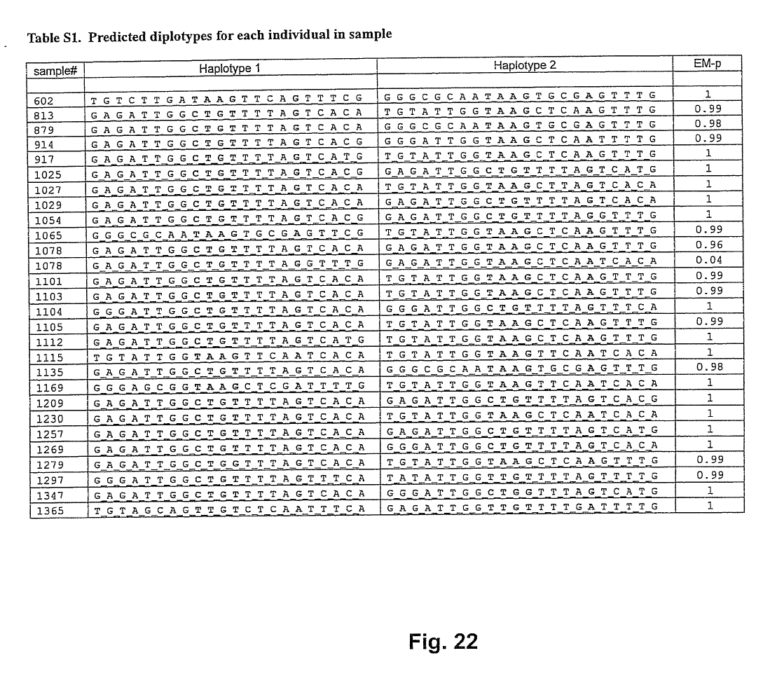

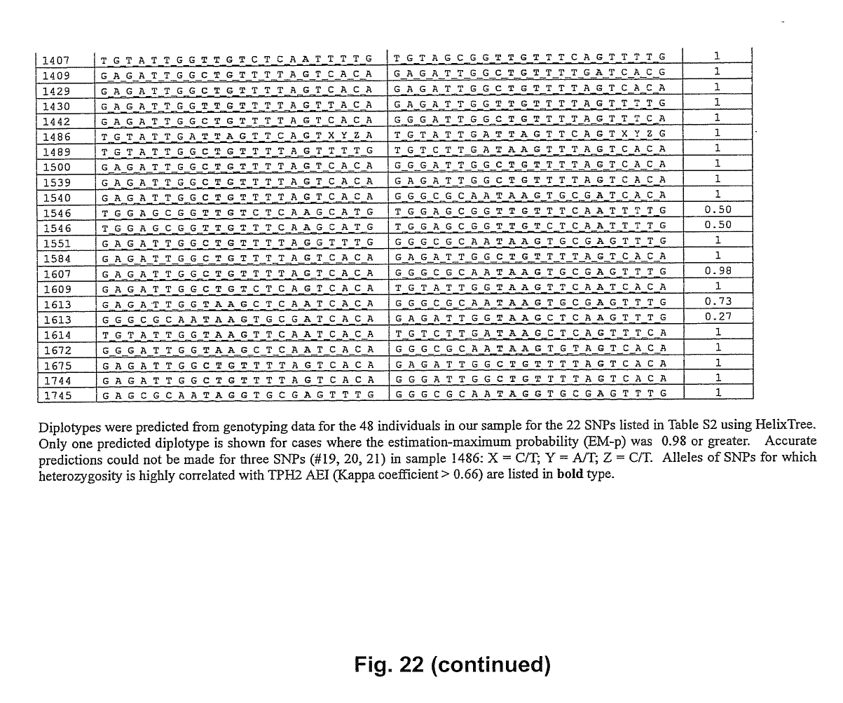

To determine whether additional SNPs correlate with TPH2 mRNA AEI, we genotyped 20 additional common TPH2 SNPs. (See Table 3 and FIG. 16 for allele frequencies and locations of these SNPs.) Alleles of each of the SNPs were in Hardy-Weinberg equilibrium in our population (not shown). FIG. 21A shows a linkage disequilibrium (D′) plot for each pair of SNPs, which was constructed from the genotyping data for the 36 Caucasians in our sample. These data show that TPH2 comprises 4 haplotype blocks: the first contains SNPs rs4570625 to rs2129575, the second rs1386488 to rs1352251, the third rs1473473 to rs9325202, and the fourth rs1487275 to rs1352252. These results are in close agreement with the haplotype structure determined from previous studies of Caucasian subjects: the HapMap CEU collection (available online at www.hapmap.org; FIG. 16) and US and Finnish populations (Zhou, Z, et al. (2005) Arch Gen Psychiatry, 62, 1109-1118) incorporated herein by reference. The frequencies of haplotypes within each block are listed in FIG. 21B, and the predicted diplotypes for each individual in our collection are listed in FIG. 22.

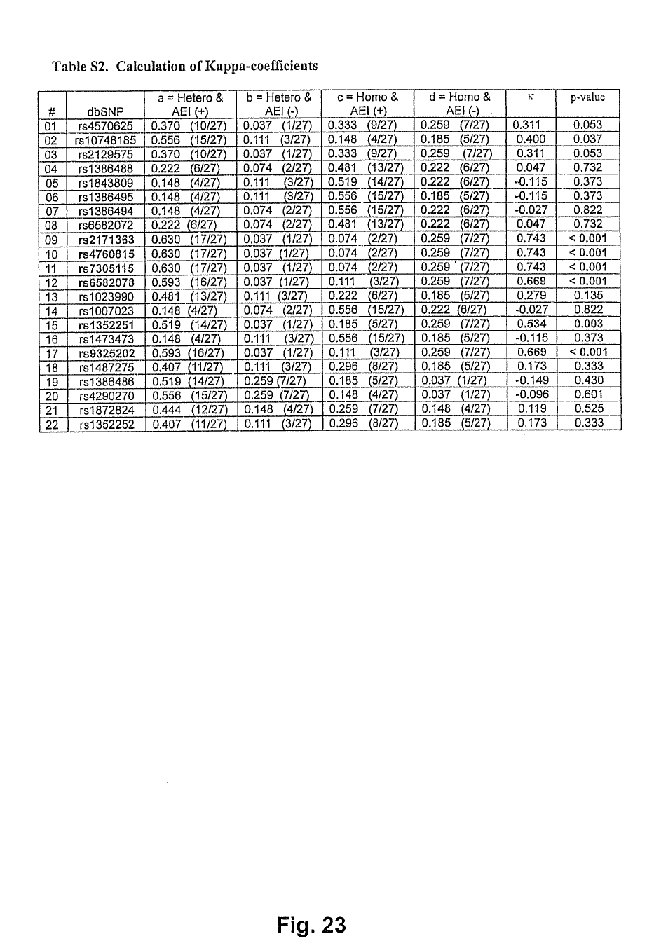

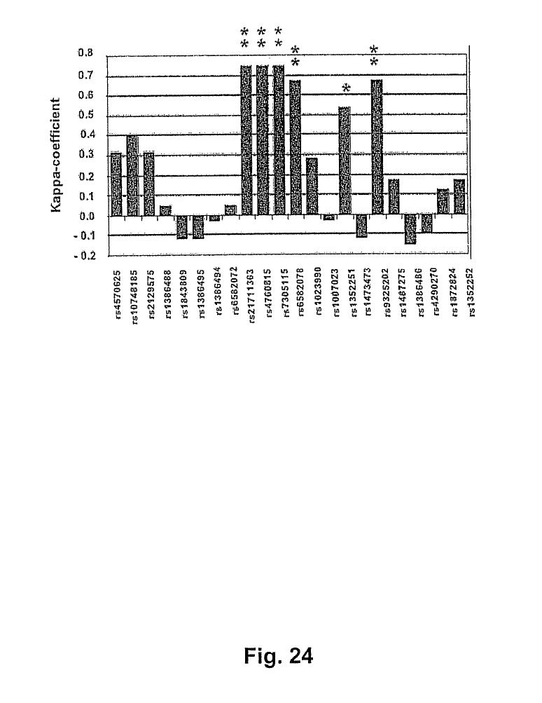

The possible contribution of each SNP to TPH2 mRNA AEI was evaluated by looking for correlations between heterozygosity/homozygosity of the SNP and the presence/absence of AEI for TPH2 mRNA within the 27 samples where AEI measurements were made. A tabulation of these results is shown in FIG. 23. The strength of each correlation was assessed using the Kappa-statistic (Saffen, D, et al. (1999) Life Sci, 64, 479-486) incorporated herein by reference. As shown in FIG. 24, five closely linked SNPs, rs2171363 (C/7), rs4760815 (T/A), rs7305115 (G/A), rs6582078 (T/G), and rs9325202 (G/A), showed statistically significant correlations with TPH2 mRNA AEI (Kappa-coefficients>0.66). Heterozygosity of rs1352251 (T/C) also correlated with TPH2 mRNA AEI (Kappa-coefficient=0.534). An independent test using a decision-tree based algorithm (Helix-Tree) found statistically significant correlations between SNP heterozygosity and AEI (P<0.01) for rs2171363, rs4760815, rs7305115, rs6582078, and rs9325202 (data not shown).

As mentioned above, AEI measurements revealed that TPH2 mRNA containing the rs7305115 A-allele is expressed at higher levels than mRNA containing the G-allele. Among 18 samples showing AEI for TPH2 mRNA, 17 were heterozygous for rs7305115 (FIG. 22). Fifteen of the 18 samples were heterozygous for the exactly complementary (i.e., “yin” and “yang”) haplotypes CTGTG and TAAGA, comprising the SNPs rs2171363, rs4760815, rs7305115, rs6582078, and rs9325202, respectively. Table 6 lists the frequencies for haplotypes containing the rs7305115 G-allele or A-allele within the Caucasian subset of our sample. These data show that G-allele haplotypes, which are associated with low TPH2 mRNA expression, are more common (0.6) than A-allele haplotypes (0.4), which are associated with high TPH2 mRNA expression. The population frequencies of the rs7305115 G- and A-alleles are similar to those previously reported for Caucasian and other populations (available online at www.ncbi.nlm.nih.gov/SNP/snp_ref.cgi?rs=7305115).

TABLE 6
Haplotype frequencies for 38 Caucasians in
sample (76 chromosomes)

| rs7305115 G-allele | rs7305115 A-allele | |
|---|---|---|
| haplotypes | haplotypes | frequency |
| C T G T G | | 0.553 (42/76) |
| | T A A G A | 0.316 (24/76) |
| | T A A G G | 0.053 (4/76) |
| | T T A G G | 0.026 (2/76) |
| T T G T A | | 0.026 (2/76) |
| C T G T A | | 0.013 (1/76) |
| C T G G G | | 0.013 (1/76) |
| Total G-allele | | 0.605 (46/76) |
| haplotypes | | |
| | Total A-allele | 0.395 (30/76) |
| | haplotypes | |

Listed haplotypes comprise the following SNPs: rs2171363 (C/T), rs4760815 (T/A), rs7305115 (G/A), rs6582078 (T/G) and rs9325202 (G/A).

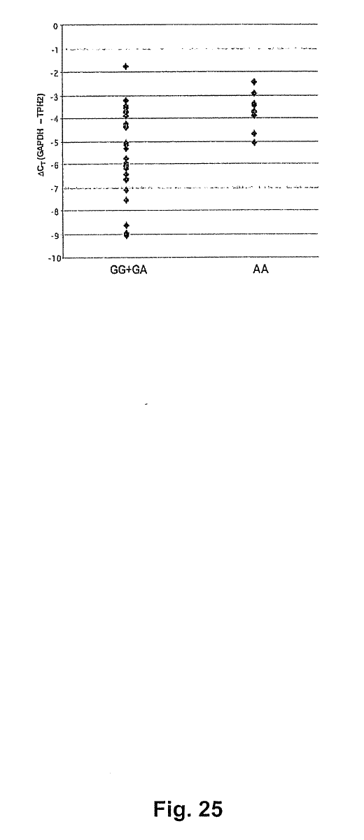

To test our ability to predict levels of TPH2 mRNA expression based upon genotype, we compared levels of TPH2 mRNA in pons samples from individuals who are heterozygous (G/A) or homozygous (G/G or A/A) for rs7305115 alleles. Real-time RT-PCR measurements of TPH2 mRNA were carried out using RNA isolated from 18 (G/G), 21 (G/A) and 9 (A/A) samples. TPH2 mRNA measurements [expressed as cycle thresholds (C_{T})] were normalized by subtracting C_{T} values for glyceraldehydes-3-phosphate dehydrogenase (GAPDH) mRNA, which is ubiquitously expressed. Pairwise comparisons between groups showed that the A/A sample contained statistically higher levels of TPH2 mRNA compared to the G/G sample (p=0.024) or the G/A sample (p=0.04). There was no statistical difference in levels of TPH2 mRNA expression between the G/G and G/A samples (p=0.659). FIG. 25 shows the distribution of TPH2 mRNA measurements for combined G/G and G/A samples compared to A/A samples. Although the spread of the data is large for both sets of samples, the A/A samples contain statistically significant higher levels of TPH2 mRNA compared to the combined G/G and G/A samples (p=0.0075). Cycle thresholds for GAPDH varied from 15 to 18.4.

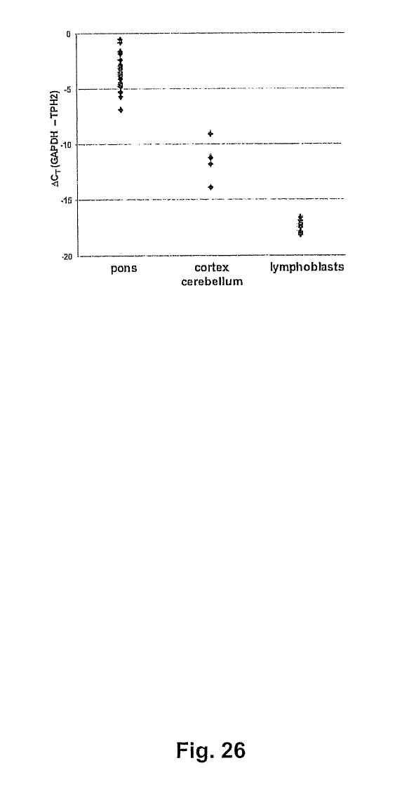

To address the question whether mRNA levels in the pons tissue sections reflect specific expression in serotonergic neurons, rather than nonspecific background expression, we compared TPH2 mRNA levels in pons with levels in cerebellum and cortex and lymphoblasts. Again, GAPDH mRNA was used as a reference. As shown in FIG. 26, TPH2 mRNA levels were significantly higher in pons compared to cerebellum, occipital, frontal, parietal or temporal cortex and much higher than levels in lymphoblasts (ANOVA; p<0.0001).

Discussion

This study is the first to reveal the presence of a frequent, functional, cis-acting polymorphism in the TPH2 gene that significantly affects mRNA expression. To detect allelic differences in TPH2 mRNA expression, we developed and validated an accurate assay of allelic expression imbalance (AEI) applicable to human autopsy brain tissues. Importantly, the functional analysis was performed in human pons, the physiologically relevant target tissue. Allelic differences in TPH2 mRNA levels likely reflect expression in serotonergic neurons in the dorsal and median raphe nuclei, which are the primary source of serotonin in forebrain. Genotyping SNPs located within the TPH2 gene identified individual SNPs and haplotypes that predict high or low levels of TPH2 mRNA expression in human pons (FIG. 24). Specifically, low levels of TPH2 mRNA expression are associated with the CTGTG combination of alleles and high levels of expression with the TAAGA combination of alleles for the SNPs rs2171363, rs4760815, rs7305115, rs6582078 and rs93252002.

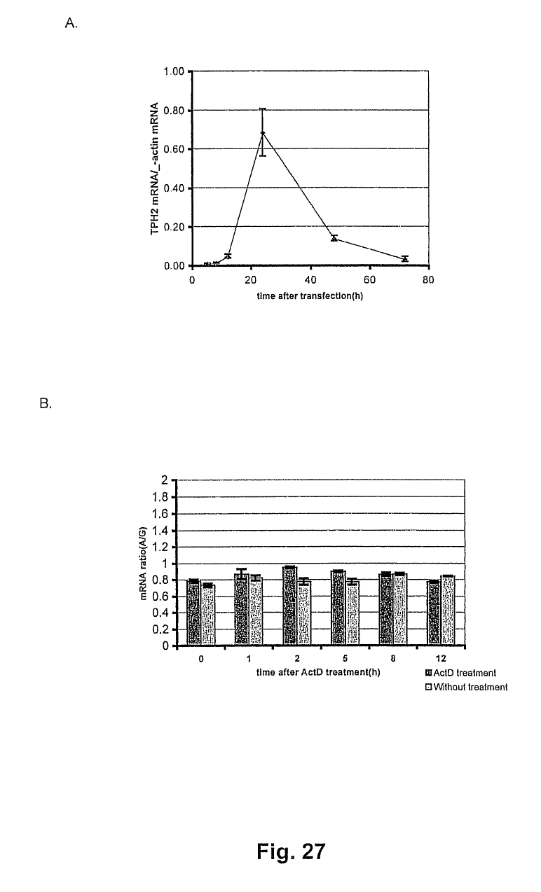

Because these SNPs are tightly linked (FIG. 16), it is not evident which SNP or SNP-combination is the “functional” element that controls TPH2 mRNA levels. Four of these SNPs (rs2171363, rs4760815, rs6582078 and rs93252002) are located within introns and one (rs7305115) within a coding exon. Analysis of predicted changes in mRNA structure for each of these SNPs using Mfold (Zuker, M, (2003) Nucleic Acids Res, 31, 3406-3415) showed only small differences between alleles (A. Johnson, data not shown). To investigate possible functional effects of the exonic SNP rs7305115, we exogenously expressed TPH2 mRNA of both alleles in CHO cells using cDNA expression vectors. No prominent differences in allelic expression or mRNA degradation rates were detectable between exogenously expressed TPH2 mRNAs containing the rs7305115 A- or G-allele (FIG. 27). This result however does not address possible differences in mRNA processing and maturation occurring at the level of hnRNA, since introns were absent from the cDNA constructs.

Analysis of possible effects of TPH2 SNPs on mRNA transcription and processing using the webtool PupaSNP (available online at pupasnp.bioinfo.ochoa.fib.es; Conde, L, et al. (2004) Nucleic Acids Res, 32, W242-248) each incorporated herein by reference, showed that the A-allele of rs7305115 (the minor allele) generates a consensus binding site for the serine-arginine (SR)-proteins SR35 and SRP40, splicing factors that bind exonic splicing enhancers (ESEs) (Cartegni, L, et al. (2002) Nat Rev Genet, 3, 285-298) incorporated herein by reference.

Exons containing a nonfunctional or partially functional ESE are often skipped during RNA splicing, possibly accounting for the lower yield of mRNA from the G-allele, which appears to be the main ancestral allele (see below). Skipping of exon 7 of the TPH2 gene would result in a modified mRNA that encodes a truncated form of TPH2 due to the insertion of an in-frame stop codon (data not shown). Recent studies have shown that mRNAs containing premature translation termination signal often undergo preferential degradation via a poorly understood mechanism termed nonsense-mediated mRNA decay (Cartegni, L, et al. (2002) Nat Rev Genet, 3, 285-298) incorporated herein by reference. Thus, the G-allele of rs7305115 might be expected to produce lower levels of full length TPH2 mRNA by increasing the frequency of exon skipping. This mechanism could account for the observed AEI of TPH2 mRNA in A/G heterozygotes (FIGS. 18-20) and lower levels of TPH2 mRNA expression in G/A heterozygotes and G/G homozygotes compared to A/A homozygotes (FIG. 25).

To determine if aberrant TPH2 mRNAs lacking exon 7 are expressed in pons, we carried out RT-PCR amplification of TPH2 cDNA using sets of synthetic oligonucleotide primers that specifically amplify cDNA segments that contain or lack exon 7, respectively. These measurements produced two PCR products, with sizes corresponding to exon 7-containing and exon 7-deleted cDNAs, in each of the 48 samples in our collection. The predicted structures of both PCR products were confirmed by DNA sequencing. Real-time PCR measurements using primer sets specific for each mRNA showed that relative levels of the full-length and exon 7-deleted forms of TPH2 mRNA varied widely between samples (data not shown). Exon 7-deleted mRNA appears to be present at very low levels, impeding a quantitative analysis. Nevertheless, these experiments provide evidence for aberrant splicing of the TPH2 gene in the pons and suggest a possible mechanism by which the rs7305115 A-allele increases and the G-allele decreases levels of TPH2 mRNA. Our results suggest that the A-allele may yield higher mRNA levels by enhancing the efficiency of proper mRNA splicing, representing a gain-of function.

The rs7305115 G-allele appears to be the ancestral allele, since sequences from a rhesus monkey (available online at www.hgsc.bcm.tmc.edu/projects/rmacaque/ and a chimpanzee (available online at www.hgsc.bcm.tmc.edu/projects/chimpanzee/) have G at this position. The G-allele is also present in the mouse and rat. The high frequency of the A-allele in Caucasian populations (0.33 to 0.41) could have resulted from a population bottleneck or random genetic drift, or by positive selection. Since the A-allele is also present at high frequency (0.29 to 0.39) in African populations, it dates back to early human evolution. The high accumulation of a gain-of-function polymorphism is unusual and point towards positive selection, or balanced selection. The existence of positive selection would indicate that TPH2 variants significantly affect reproduction, possibly through a positive effect on mood or mental activity.

Even before the functional element(s) that control levels of TPH2 mRNA expression are identified, knowledge of marker SNPs and haplotypes that strongly predict high or low levels of TPH2 mRNA expression should be useful for association studies seeking to establish a role for TPH2 in human disease. Because TPH2 encodes the enzyme that catalyzes the rate-limiting step in the synthesis of serotonin, it is plausible that differences in TPH2 mRNA expression in the range of 1.2 to 2.5-fold could contribute to disorders in which serotonin plays a role. Moreover, the high frequencies of the implicated SNPs and haplotypes suggest a possible role in brain disorders that affect a significant portion of the population, such as major depression, which has a life-time prevalence of about 16%.

A study by Zhou and coworkers (Zhou, Z, et al. (2005) Arch Gen Psychiatry, 62, 1109-1118; incorporated herein by reference) examined associations between 15 TPH2 SNPs and: 1) anxiety/depression, 2) suicide attempt, and 3) major depression in four populations. Weak associations between these disorders and individual SNPs located within the intron 5 to intron 8 segment of TPH2 were observed. The SNPs showing associations, however, varied between disorders and between populations, and none remained significant after correction for multiple testing. Haplotype analysis revealed the presence of high-frequency “yin” and “yang” haplotypes, with complementary patterns of major and minor alleles. Again, weak associations (significant only in the absence of corrections for multiple testing) were observed, with a trend towards association of the yin-haplotype (the major allele which includes the G-allele of rs7305115) with anxiety/depression and suicide, and possible protection from these disorders by the yang-haplotype (which includes the A-allele of rs7305115). The yin-haplotype was also associated with lower cerebral spinal fluid levels of the serotonin metabolite 5-hydroxyindolacetic acid in non-medicated controls who were free of psychiatric disorders. Significantly, we showed in this study that the yin-haplotype associates with low levels of TPH2 mRNA expression.

As described above, we observed a positive correlation between rs4570625 heterozygosity and TPH2 mRNA AEI in adult pons (Kappa-coefficient=0.311; p=0.053; FIGS. 23-24). These data suggest that rs4570625 does not control TPH2 mRNA expression, but may be in partial linkage disequilibrium with a functional polymorphism that does. In fact, our genotyping results (FIG. 21) predict that rs4570625 is in partial linkage disequilibrium with SNPs (rs2171363, rs4760815, rs7305115, and rs6582078) that highly correlate with TPH2 mRNA AEI. These observations suggest that re-analysis of the imaging and electroencephalography data in the above studies might show stronger correlations with rs7305115 compared to rs4570625. Alternatively, it is possible that rs4570625 (or a closely linked polymorphism in the promoter region) directly regulates TPH2 mRNA expression specifically during times of emotional stress and/or during brain development. Since serotonin has been shown to play a role in the development of the brain, it is possible that differential expression of THP2 at specific stages of brain development may differentially influence the development of neuronal circuits that control amygdala activity in the adult. This

Previous studies of various SNP's in TPH2 provide preliminary evidence for a role for TPH2 alleles in several mental disorders and processing of emotional stimuli, including major depression, bipolar disorder, suicidal behavior, anxiety, ADHD, and autism. None of the previous studies, however, identified functional alleles, and thus do not provide mechanistic explanations for the observed associations. The fact that many of these studies identified different associating SNPs suggests that the studies may lack sufficient power to reliably detect associations for SNPs that are in partial linkage with a functional polymorphism within the TPH2 gene. We predict that larger studies would show stronger associations for most SNPs in the region, with the strongest association observed for the functional polymorphism.

In this study, we scored heterozygous SNPs as being positively correlated with AEI, if the measured AEI was greater than 1.2. Perhaps stronger associations with mental illness could be detected using combinations of SNPs that predict higher levels of AEI, e.g., greater than 2. The gain-of-function we have observed for the rs7305115 A-allele is likely to have a protective effect. This effect may only become apparent in combination with variants in one or more additional genes that functionally interact with TPH2. Accounting for interactions among multiple genes could reveal significant impact on mental disorders, or variation in normal human behavior.

Identifying genetic variants that modify, or strongly predict, levels of mRNA expression for candidate genes provides a rich source of markers with high “prior-probability” for association studies. In particular, using allele-specific mRNA expression as an intermediate phenotype is an efficient method for identifying “functional” polymorphisms that contribute to the complex phenotypes associated with mental illness or response to therapeutic drugs.

### Example 3

### Novel Polymorphisms in the Human Dopamine D2 Receptor Gene

Subcortical dopamine D2 receptor (DRD2) signaling has been implicated in cognitive processes and brain disorders, but the responsible DRD2 variants remain ambiguous. We measured allelic DRD2 mRNA expression in human striatum and prefrontal cortex autopsies, followed by single nucleotide polymorphism (SNP) scans of the entire gene locus. A novel promoter SNP (rs12364283) located in a conserved suppressor region was associated with enhanced DRD2 expression, whereas previously studied DRD2 variants failed to affect expression. In addition, two frequent intronic SNPs (rs2283265 and rs1076560) reduced formation of the DRD2 short splice variant relative to DRD2 long, which was reproduced in vitro using minigene constructs. In healthy human subjects, both intronic SNPs were associated with greater activity of the striatum and prefrontal cortex, assessed with fMRI during working memory—consistent with known dopamine modulation of neuronal firing via DRD2 short/long ratios. Our results identify regulatory DRD2 polymorphisms that can affect working memory pathways and risk for human brain disorders.

Introduction

Aberrant subcortical dopamine D2 receptor (DRD2) signaling has been implicated in several brain disorders, including drug addiction, schizophrenia, and Parkinson's disease. DRD2 variants, including a SNP termed Taq1A (rs1800497), a promoter region polymorphism (−141C del/ins) (rs1799732), and a synonymous SNP in exon 7 (C957T) (rs6277) have been associated with schizophrenia and drug abuse. However, these associations have not been consistently replicated, and the physiological mechanisms by which they might affect disease risk remain unknown. Our goal was to identify functional DRD2 polymorphisms and link these to a physiological function in the CNS.

Endowed with prominent DRD2 signaling, the basal ganglia represent a CNS region where DRD2 variants could have maximal functional impact. A crossroad between cortex and dopamine projections from the brainstem, basal ganglia in regions including the caudate and the pallidum are involved in cognitive processes such as working memory, contributing to the focus of working memory. DRD2 mediated dopamine signaling is a major modulator within these structures, affecting GABA and cortical glutamate signals impinging on striatal medium spiny neurons. Moreover, DRD2 density was shown to affect working memory performance in mice, while, studies in humans have demonstrated a tight relationship between striatal DRD2 receptor availability and working memory or attention. However, overall density of DRD2 is not the only mechanism modulating striatal neuronal firing. DRD2 receptors exist in two main splice variants, DRD2L (long) and DRD2S (short), including or lacking exon 6. While DRD2L is thought to reside mainly postsynaptically, DRD2S is expressed mainly presynaptically. Relative expression of DRD2S and L is critical to dopamine modulation of GABA and glutamate striatal transmission.

To identify genetic factors in DRD2 signaling, we tested previously proposed polymorphisms and searched for novel variants modulating dopamine neurotransmission. Because the DRD2 locus lacks frequent nonsynonymous SNPs that alter receptor function, we focused on regulatory polymorphisms affecting gene transcription, mRNA processing, and splicing. This was achieved with use of allelic expression analysis in human autopsy tissues from prefrontal cortex and striatum. Allelic expression imbalance (AEI) is a powerful means for detecting cis-acting regulatory polymorphisms. With this approach, we have identified a regulatory promoter SNP and two SNPs affecting DRD2 splicing, whereas previously proposed polymorphisms had no direct effects on allelic DRD2 expression. Moreover, the DRD2 variants affecting splicing to DRD2S autoreceptors are associated with differential activity in the working memory network, measured with fMRI during working memory in healthy humans.

Results

Allelic DRD2 Expression in Human Brain Tissues and SNP Scanning

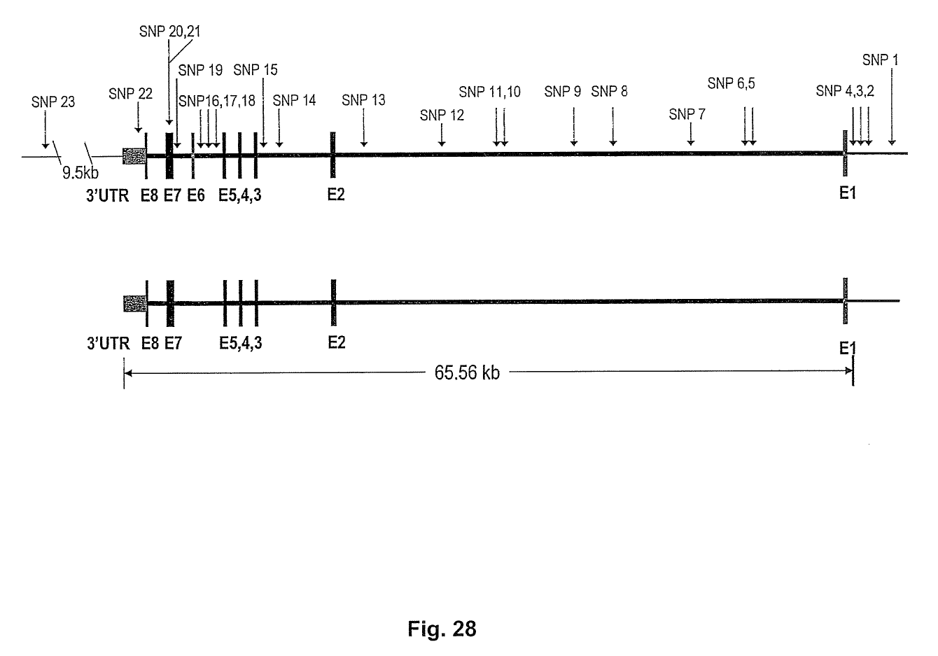

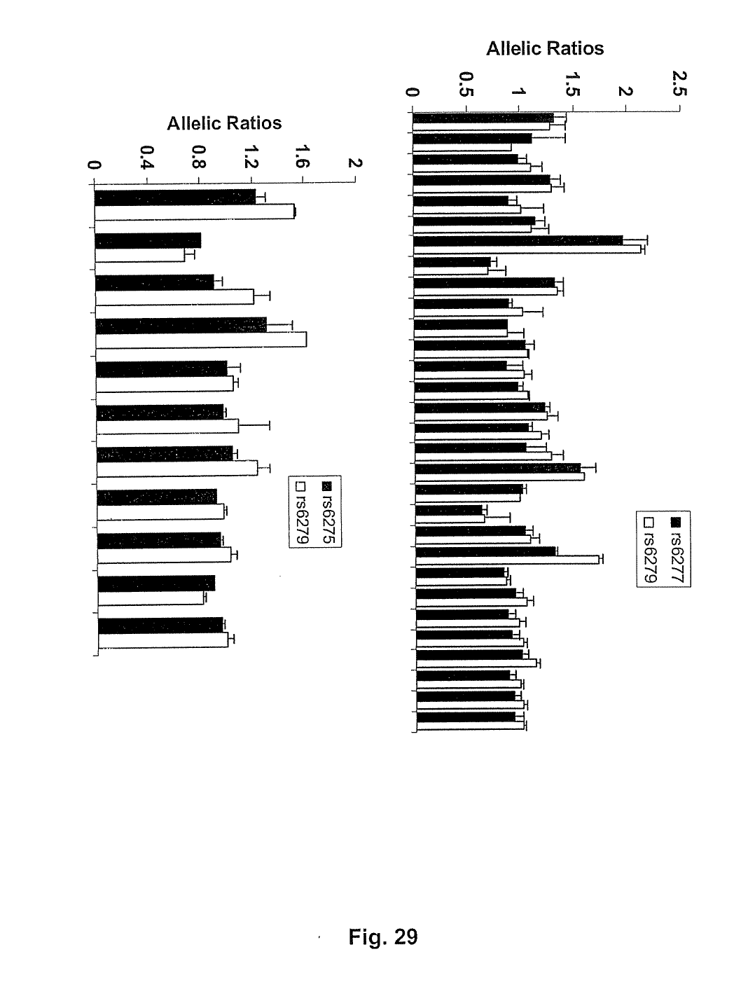

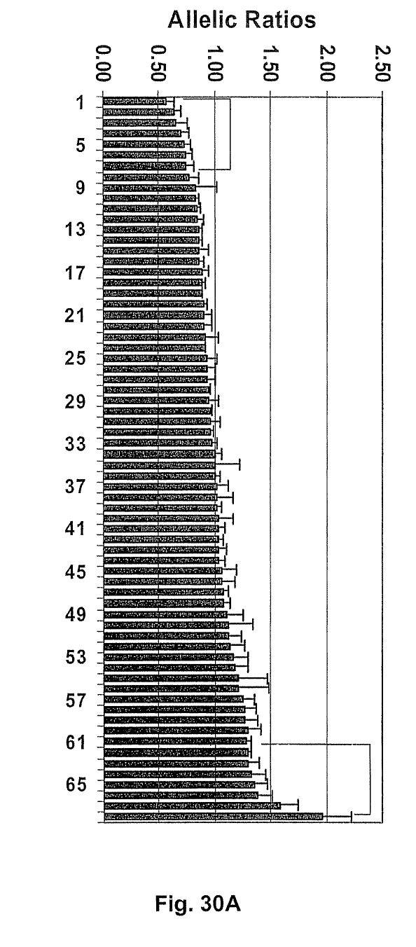

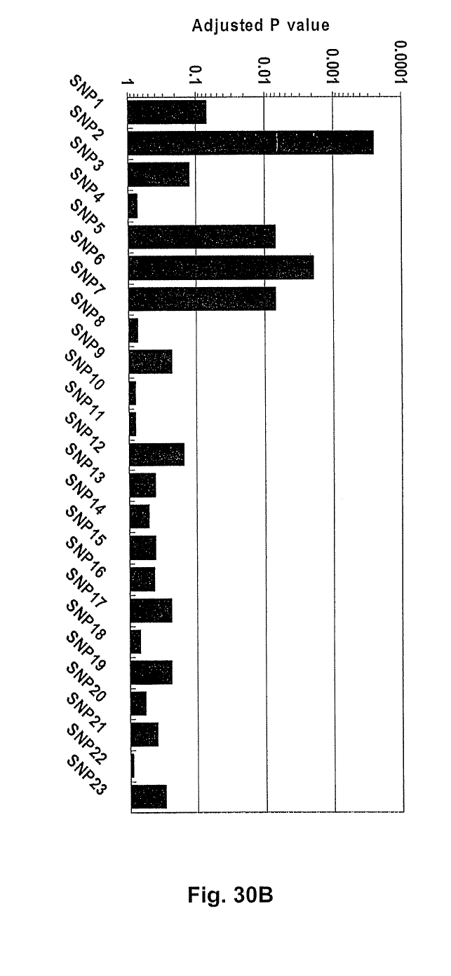

Differences in mRNA expression from each DRD2 allele (allelic expression imbalance, AEI) reveal cis-acting regulatory polymorphisms. This approach requires use of marker SNPs located in transcribed regions, to permit comparison of allelic ratios in genomic DNA and mRNA (after conversion to cDNA). Using a PCR-primer extension analysis (SNaPshot), we measured allelic DRD2 expression in 68 autopsy tissue samples (54 from prefrontal cortex, 14 from striatum) heterozygous for at least one of three marker SNPs (SNP20 and SNP21 in exon 7, and SNP22 in 3′-UTR) (Table 7, FIG. 28). Measured allelic ratios were similar when obtained independently with two marker SNPs in compound heterozygotes (r=0.93-0.96) (FIG. 29), supporting validity of the assays. Of the 68 tissues tested, 8 displayed significant AEI with ratios above unity, while 7 were below unity (FIG. 30A), suggesting presence of a regulatory polymorphism not in linkage disequilibrium with the marker SNPs.

TABLE 7
Genotyped SNPs of DRD2. Allele frequencies were calculated from the 105
samples of the Stanley Foundation (prefrontal cortex). Allele frequencies
in the cohort of 100 subjects from the University of Bari are also provided
where available.

| SNP | | | positions | | MAF | |
|---|---|---|---|---|---|---|
| number | SNP | | (genome) | regions (gene) | Stanley F. | MAF Univ. Bari |
| 1 | rs10891556 | G/T | 112857971 | 5′upstream | 0.15 | |
| 2 | rs12364283 | T/C | 112852165 | 5′upstream | 0.07 | 0.09 |
| 3 | rs1799978 | A/G | 112851561 | promoter | 0.05 | |
| 4 | rs1799732 | C/−C | 112851463 | promoter | 0.08 | |
| 5 | rs4938019 | T/C | 112846601 | intron 1 | 0.14 | |
| 6 | rs4350392 | C/A | 112840927 | intron 1 | 0.13 | |
| 7 | rs4648317 | C/T | 112836742 | intron 1 | 0.14 | |
| 8 | rs4581480 | T/C | 112829684 | intron 1 | 0.08 | |
| 9 | rs12574471 | C/T | 112821446 | intron 1 | 0.14 | |
| 10 | rs4648318 | A/G | 112818599 | intron 1 | 0.25 | |
| 11 | rs7125415 | C/T | 112815891 | intron 1 | 0.08 | |
| 12 | rs7103679 | C/T | 112808884 | intron 1 | 0.15 | |
| 13 | rs1125394 | A/G | 112802395 | intron 1 | 0.17 | |
| 14 | rs2734836 | G/A | 112796449 | intron 2 | 0.16 | |
| 15 | rs2075654 | G/A | 112794276 | intron 2 | 0.18 | |
| 16 | rs12363125 | A/G | 112791126 | intron 5 | 0.41 | |
| 17 | rs2283265 | G/T | 112790746 | intron 5 | 0.17 | 0.10 |
| 18 | rs2511521 | T/C | 112790509 | intron 5 | 0.24 | |
| 19 | rs1076560 | G/T | 112788898 | intron 6 | 0.17 | 0.12 |
| 20 | rs6275 | C/T | 112788687 | exon 7 | 0.28 | |
| 21 | rs6277 | C/T | 112788669 | exon 7 | 0.46 | |
| 22 | rs6279 | C/G | 112786283 | 3′UTR | 0.28 | |
| 23 | rs1800497 | C/T | 112776038 | 3′downstream | 0.18 | |

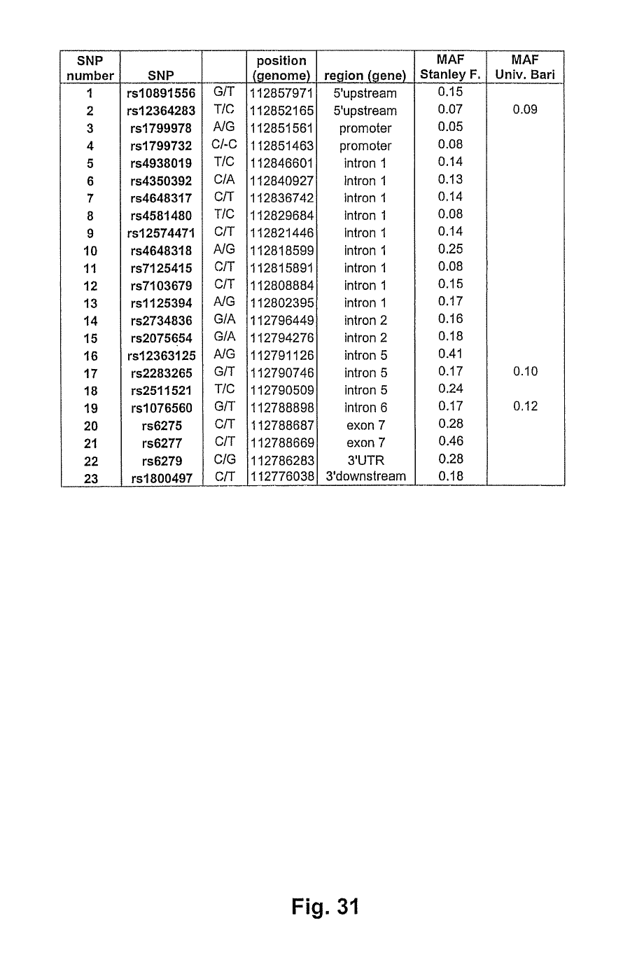

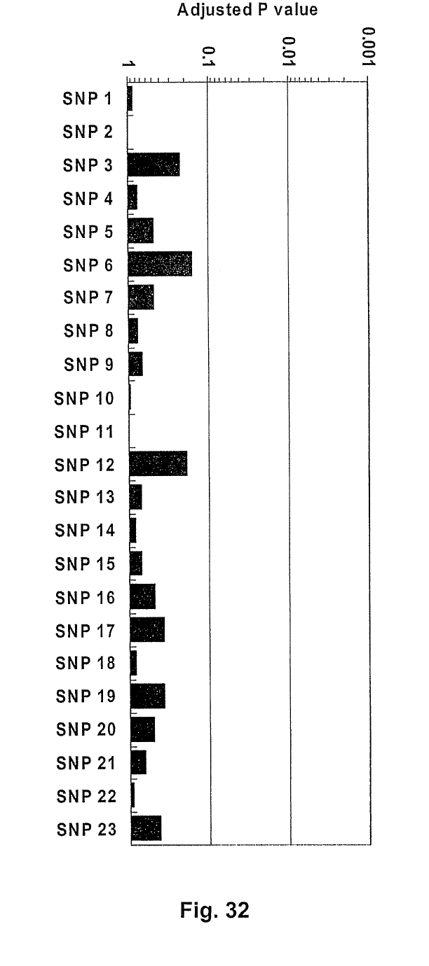

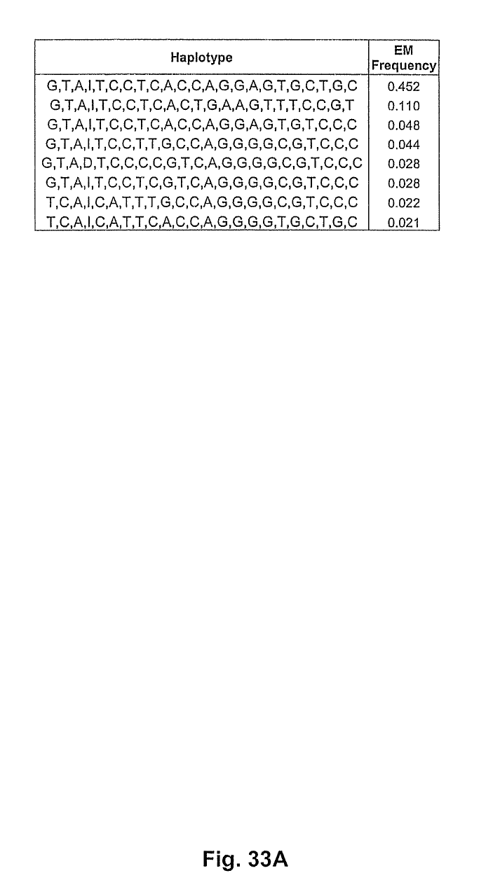

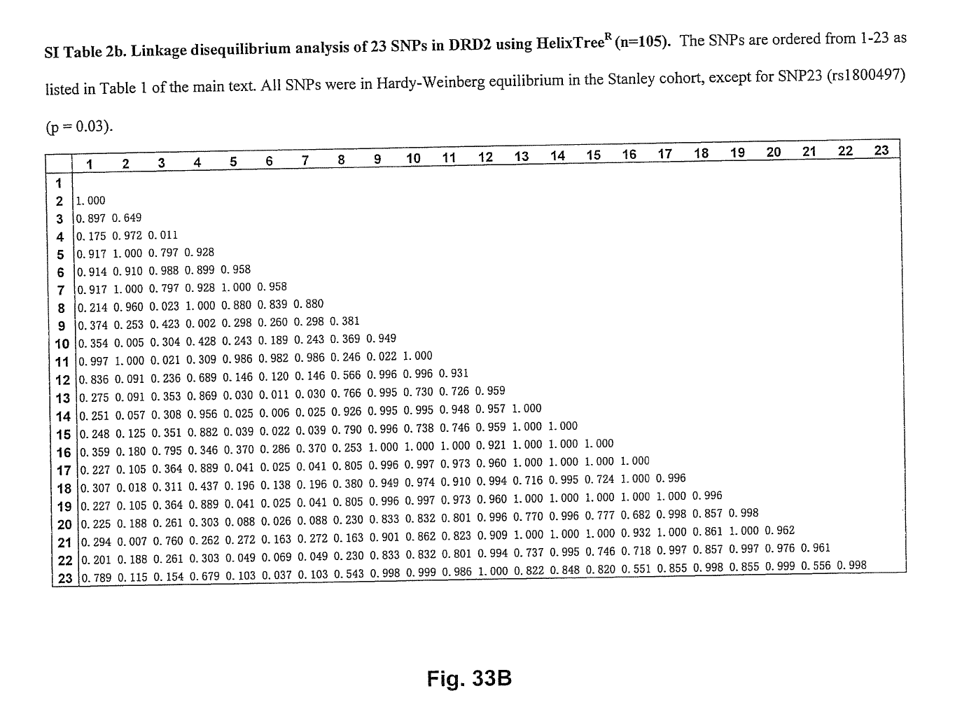

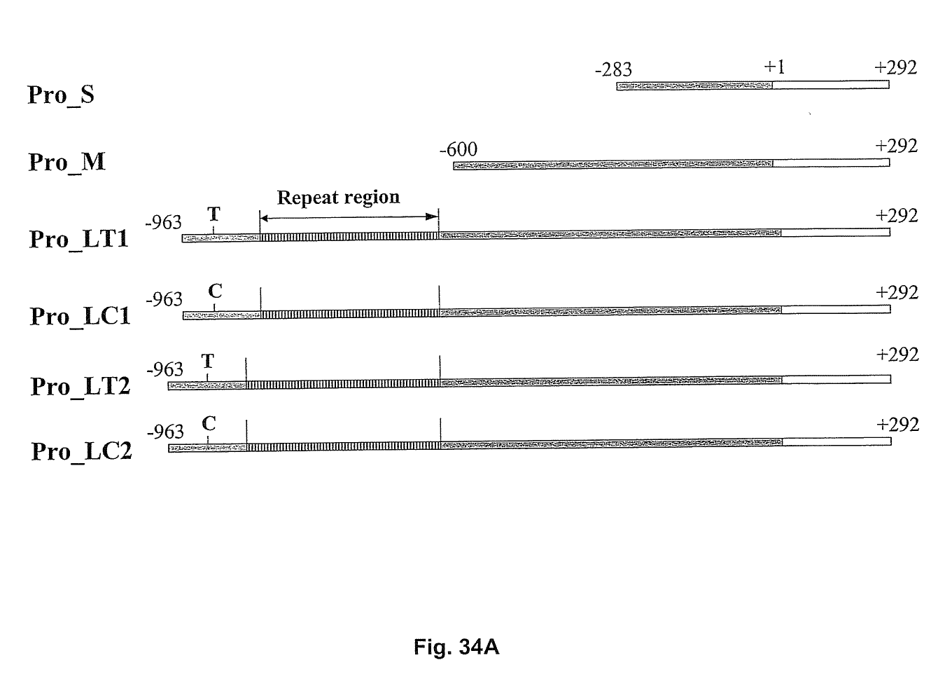

To search for functional DRD2 polymorphism, we genotyped 23 SNPs in all samples (SNP1-23; Table 7). Predicted haplotype frequencies and pairwise linkage D′ scores (FIG. 31) are consistent with previous studies, showing strong haplotype block 3′-downstream from exon 2, whereas linkage disequilibrium is low in intron 1 and 5′-upstream. Scanning the DRD2 locus with AEI as phenotype revealed a strongly linked promoter region SNP (SNP2; p=0.001, adjusted p=0.023) (Table 8). A two-loci genetic plot analysis with HelixTree™ (Golden Helix) did not further strengthen the association, suggesting that SNP2 is the sole polymorphism contributing to AEI (adjusted P=9.74×10⁻⁵, FIG. 30B). Moreover, removal of subjects heterozygous for SNP2 from the pool failed to reveal a significant association of any other SNPs with the remaining subjects showing AEI (FIG. 32). Specifically, marker SNP21, suggested to affect mRNA stability in vitro (Duan, J., et al. (2003) Hum Mol Genet. 12, 205-16), and the newly discovered putative regulatory SNP14 in intron 2 (Rogaeva A, et al., (2007) J Biol. Chem. 282:20897-20905) each incorporated herein by reference, were not associated with the observed allelic ratios (FIG. 30B). In addition to these SNPs, we identified 10 variants of different length within a GAA/GAAA repeat region located 3′ of SNP2 in 105 prefrontal cortex samples (FIG. 33). These variants were also not significantly correlated with AEI (adjusted P=0.31, HelixTree™), indicating that the GAA/GAAA repeat region does not critically affect transcription in human brain tissues.

TABLE 8
Association of SNP2 (rs12364283) with AEI in DRD2 mRNA.

AEI

| SNP2 | | 0 | 1 | Total |
|---|---|---|---|---|
| Genotype | TT | 51 | 9 | 60 |
| | TC | 2 | 6 | 8 |
| Total | | 53 | 15 | 68 |

Fisher's Exact test, P = 0.001, adjusted p-value = 0.023 (n = 68),
0 = no AEI.
1 = with AEI.

Overall mRNA expression levels did not correlate with genotype of any tested SNP (data not shown), probably because of greater noise observed in total mRNA levels compared to allelic expression ratios. Taken together, these results indicate that SNP2 (rs12364283) in position −844 of the promoter region is associated with differential expression of DRD2 mRNA.

Reporter Gene Assay Testing Promoter SNP2 (rs12364283)

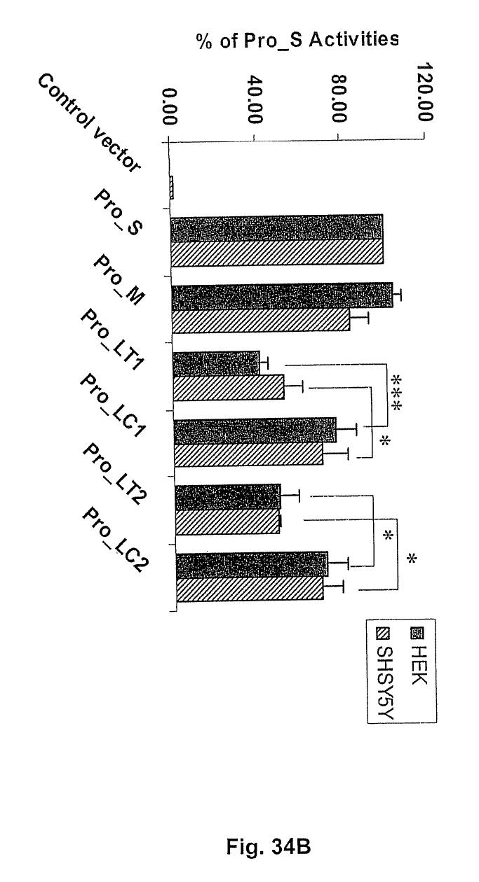

We tested DNA fragments of different lengths in the promoter region of DRD2 (Pro_S, Pro_M, Pro_L) in a luciferase reporter plasmid (FIG. 34A). Only the long fragment (Pro_L) contained SNP2 (T/C alleles, −844) and the GAA/GAAA repeat region located 3′ of SNP2. We constructed 4 different Pro_L variants containing the C or T alleles of SNP2, and two of the repeat variants (variant 1: 360 nucleotides; variant 2: 364 nucleotides) (FIG. 34A). Shown in FIG. 34B, the short and medium size promoters Pro_S and Pro_M displayed the highest promoter activities, while the Pro_L fragments were significantly less active compared to Pro_S in both cell lines tested, indicating a silencer domain resides in the region −600 to −963. However, the C allele (minor) of SNP2 significantly enhanced promoter activity over the T allele in both cell lines tested, demonstrating a disinhibitory effect of the C allele. There were no significant differences between the GAA/GAAA repeat variants, consistent with undetectable effects on DRD2 allelic ratios. These results demonstrate that promoter SNP2 affects DRD2 expression, the minor C allele displaying greater expression.

Expression of DRD2 Splice Variants in Prefrontal Cortex and Striatum

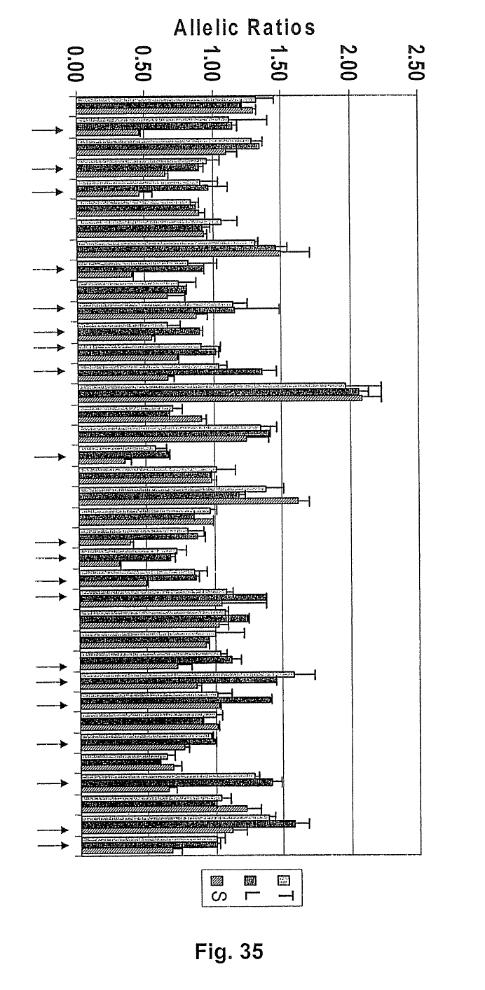

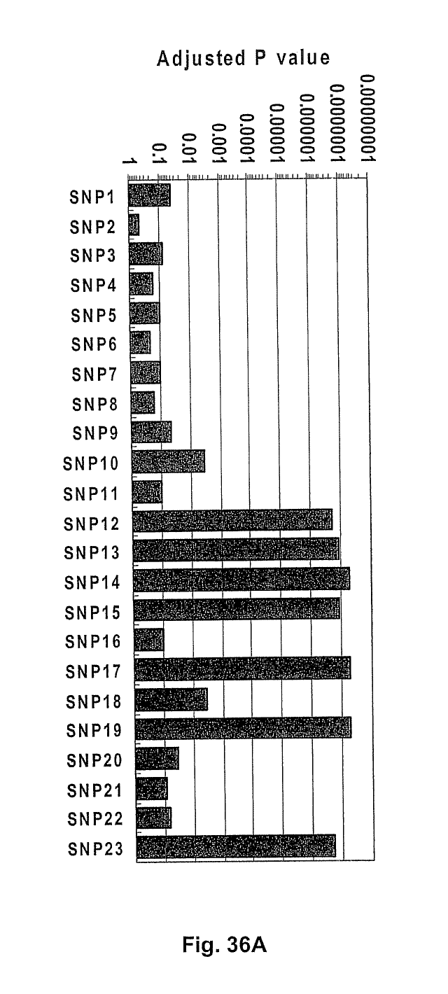

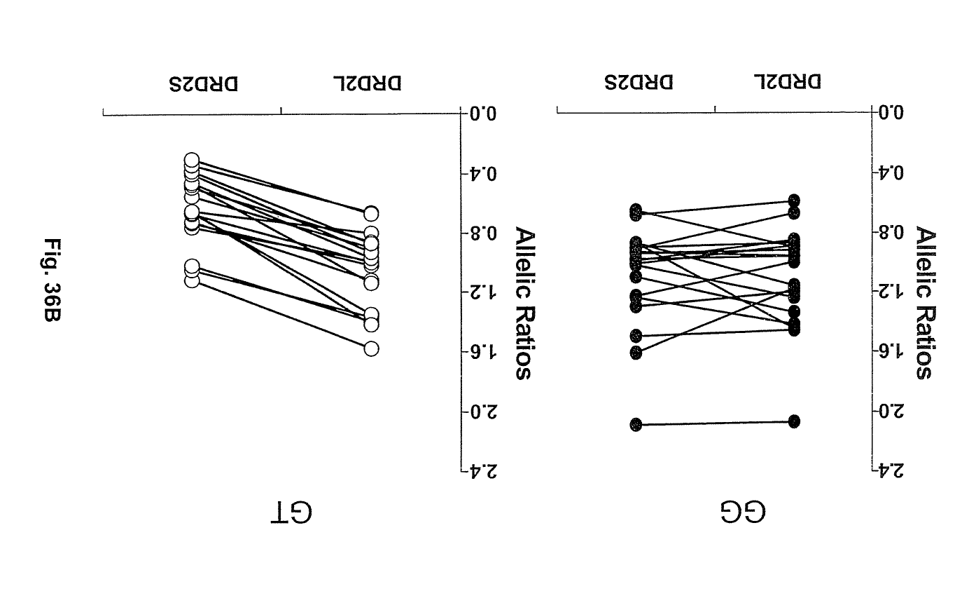

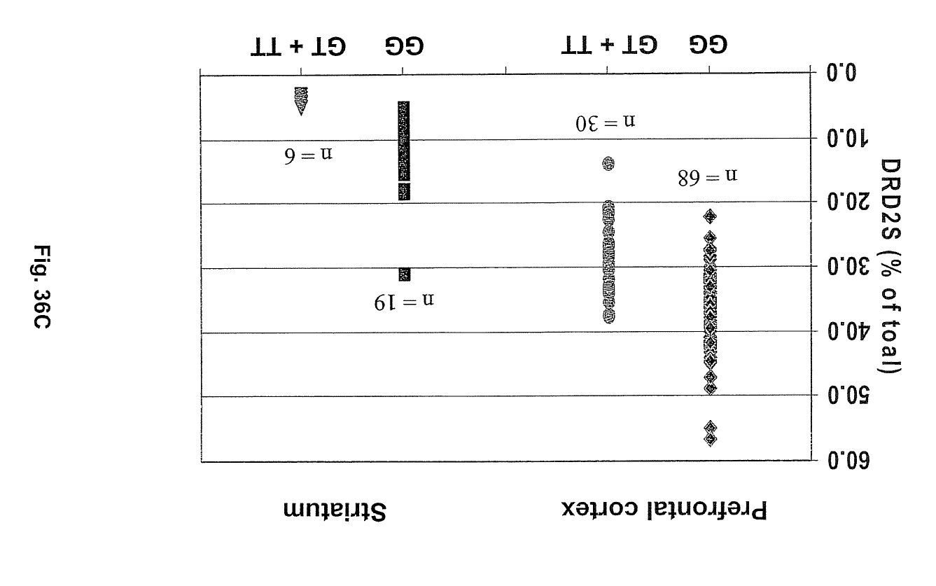

To test whether DRD2 variants affect mRNA splicing, we measured allele-specific expression for each splice variant (DRD2L and DRD2S) (FIG. 35). Allelic mRNA ratios of the two splice variants were consistent with the mean allelic ratios measured independently for total DRD2 mRNA (considering the relative abundance of S and L splice variants), supporting the accuracy of the assays. Of 37 tissues analyzed, 20 displayed substantial differences in allelic mRNA expression ratios between DRD2S and DRD2L, indicating the presence of a frequent polymorphism affecting splicing.

Scanning the DRD2 gene locus for SNPs linked to allelic splicing differences revealed a strong association with several SNPs in a large 3′-haplotype block, including SNP12-15, SNP17, SNP19, and SNP23, with SNP17 and SNP19 displaying the lowest p value (adjusted p=5.2×10⁻⁸, FIG. 3a). The location of SNP17 and 19 in introns 5 and 6 is consistent with a role in exon 6 splicing. Being in complete linkage disequilibrium in prefrontal cortex tissues (n=105, minor allele frequency 17%), this analysis does not distinguish between any role of SNP19 and SNP17 in splicing. Also located in intron 5, SNP16 and 18 were not significantly associated with splicing (FIG. 36A) and therefore not further studied. Tissues heterozygous for SNP17/19 yielded significantly lower allelic ratios for DRD2S compared to DRD2L (FIG. 36B). Considering the linkage relationship between SNP17/19 and marker SNP21, this result indicates that the minor T alleles of SNP17/19 favor a splicing process including exon 6. Therefore, the minor T alleles of the two intronic SNPs are linked to a significant reduction of the DRDR2S splice variant.

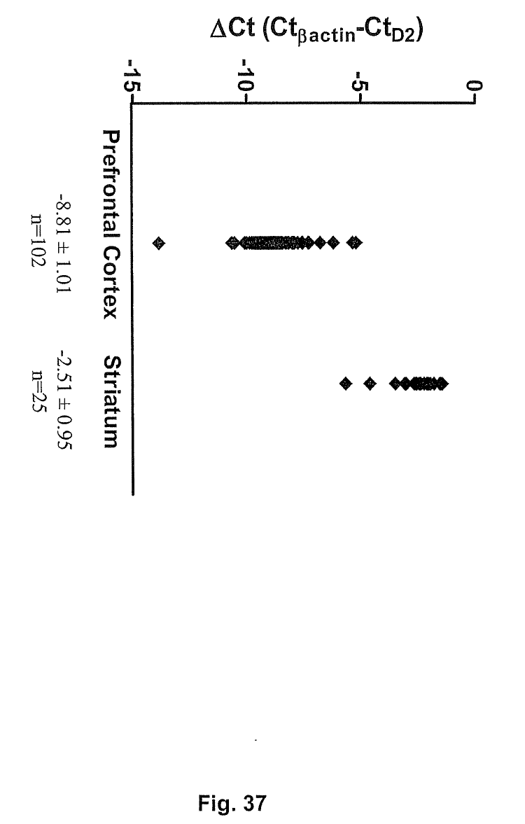

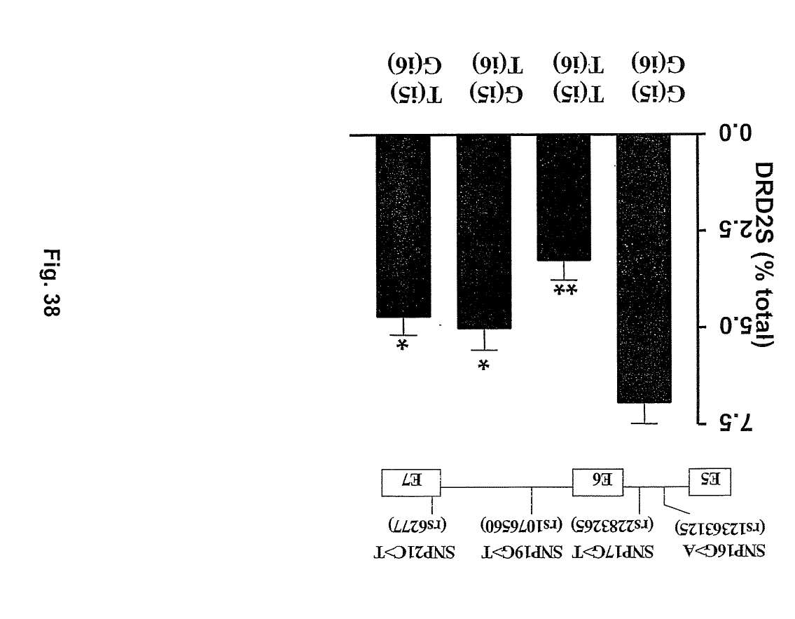

To confirm this result, we measured the expression of DRD2S and DRD2L in prefrontal cortex and striatum, using a fluorescence PCR assay that does not discriminate between the alleles. Comparable to previous studies, relative DRD2S mRNA expression was higher in prefrontal cortex (34±7%, n=98) than in striatum (9.5±6.1%, n=25) (p<0.0001 (student's t-test, SPSS). Grouping of the subjects by genotype (GG vs. GT+TT of SNP17/19) revealed that T-carriers had significantly less relative DRD2S mRNA expression than GG carriers, both in prefrontal cortex and in striatum (FIG. 36C), confirming the results obtained with allele-specific analysis of the splice variants. Overall DRD2 mRNA levels were considerably higher in striatum compared to prefrontal cortex (FIG. 37) but were not significantly different when grouped by SNP17/19 genotype (data not shown).

Genotype Effect on Alternative Splicing in a DRD2 Minigene

To study the splicing process, we constructed a partial DRD2 gene, containing introns and exons, in an expression vector (FIG. 38). Four minigene constructs carrying all possible allele combinations for SNP17/19 (G-G, T-T, T-G, and G-T) were transfected into HEK-293 cells, and percentage DRD2S of total DRD2 mRNA levels was determined. As in brain tissues, the minigene carrying the minor T alleles for both intronic SNPs generated significantly less DRD2S than the G allele (FIG. 38). However, presence of only one T allele in either of the two intronic SNPs also significantly reduced the formation of DRD2S compared to the G-G haplotype, indicating that both SNPs affect splicing. Relative expression of the short variant was lower in transfected HEK-293 cells than observed for DRD2 in brain tissues, indicating tissue-specific differences. We did observe the formation of further splice isoforms in addition to S and L in HEK-293 cells, both from endogenously expressed DRD2 and from the transfected minigenes (measured with PCR primers specific for each). The splice patterns of endogenous DRD2 and those generated from the minigene were similar, suggesting that the minigene construct contains the needed elements for cis-regulation of splicing. The significance of additional splice variants in non-neuronal tissues remains to be studied. These results support the hypothesis that both minor alleles of SNP17 and 19 contribute to reduced formation of DRD2S.

Association Studies with Working Memory

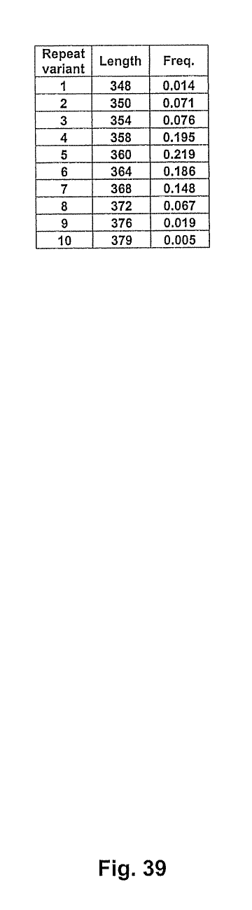

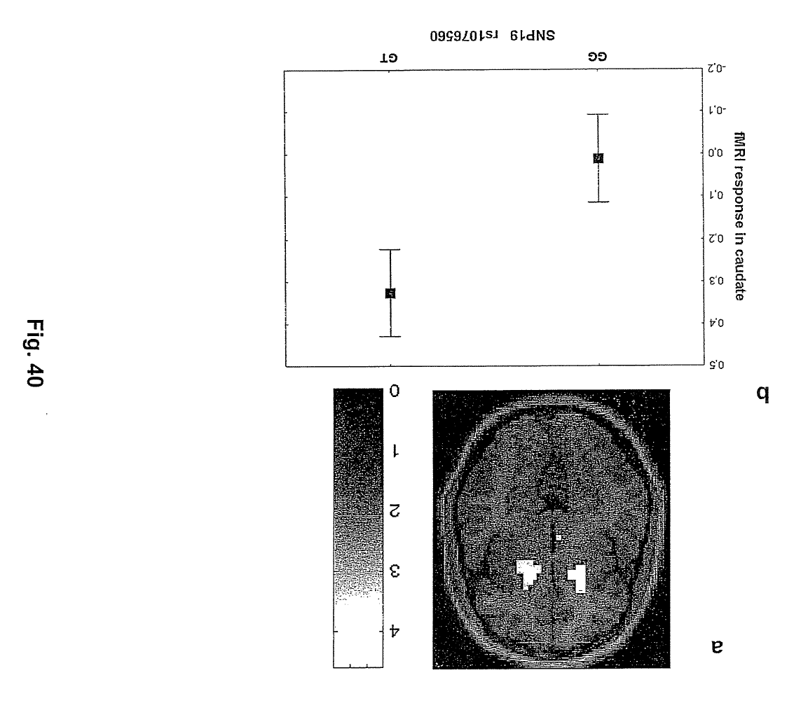

We next tested the hypothesis that the newly discovered DRD2 SNPs have physiological relevance. This was accomplished with use of fMRI measurements in normal human subjects undergoing a memory task, with particular attention to striatal regions where DRD2 signaling is prominent. For each of the three SNPs (SNP2, SNP17, and SNP19), we identified heterozygous carriers from 100 probants, and matched these with an equal number of subjects homozygous for the main allele. Allele frequencies were slightly different in this cohort (e.g., 12% for SNP19; Table 7), and none of the subjects were homozygous for the minor alleles in this group. ANOVAs showed no significant differences between genotype groups in any demographical variable (all p>0.1) (FIG. 39). Similarly, ANOVAs on behavioral data did not show any statistically significant main effect of genotypes on accuracy and response time (all p>0.1).

Functional Imaging Data and DRD2 Genotype Effect

Analysis of the working memory fMRI imaging data in the whole sample revealed significant Blood Oxygen Level Dependent (BOLD) responses in the working memory cortical and subcortical network, including dorsolateral prefrontal cortex (BA 9), anterior cingulate (BA 24 and BA 32), premotor area (BA 6), parietal cortex (BA 39/40), caudate, and putamen, consistent with earlier reports.

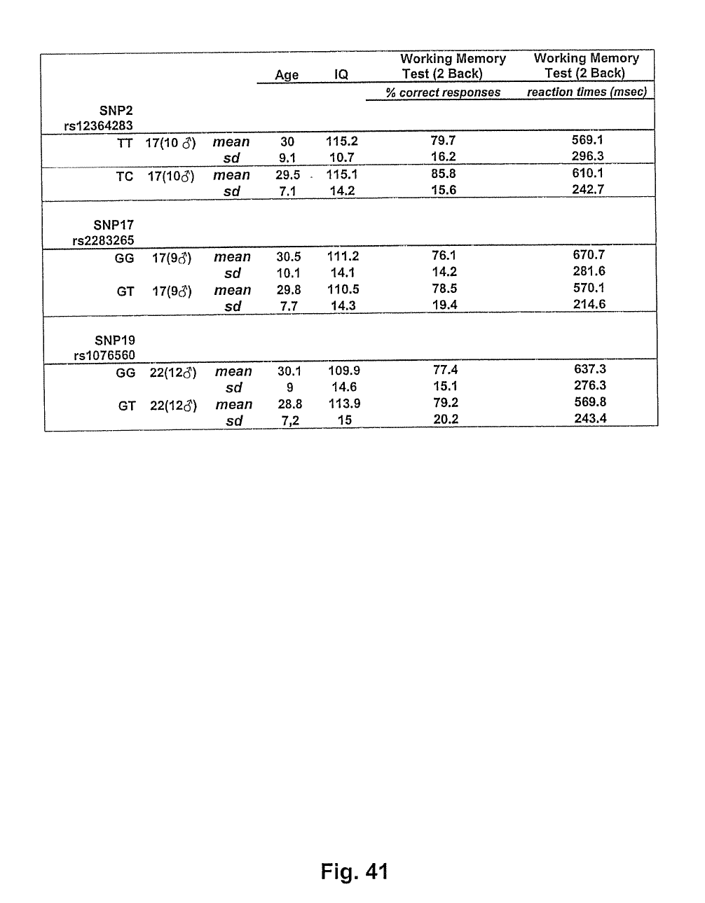

Testing for genotype effects, ANOVA of the promoter SNP2 did not indicate any statistically significant difference in any brain region. On the other hand, ANOVA of the fMRI data did show a highly significant effect of intron 6 SNP19 genotype: the G/T genotype was associated with greater BOLD activity than the GG genotype in several brain regions (FIGS. 40-41), including bilateral head of the caudate, left middle frontal gyms, left precentral gyms, left anterior cingulate, left thalamus, left superior frontal gyms and left caudate tail. The opposite contrast (G/G>G/T) did not show any significant difference.

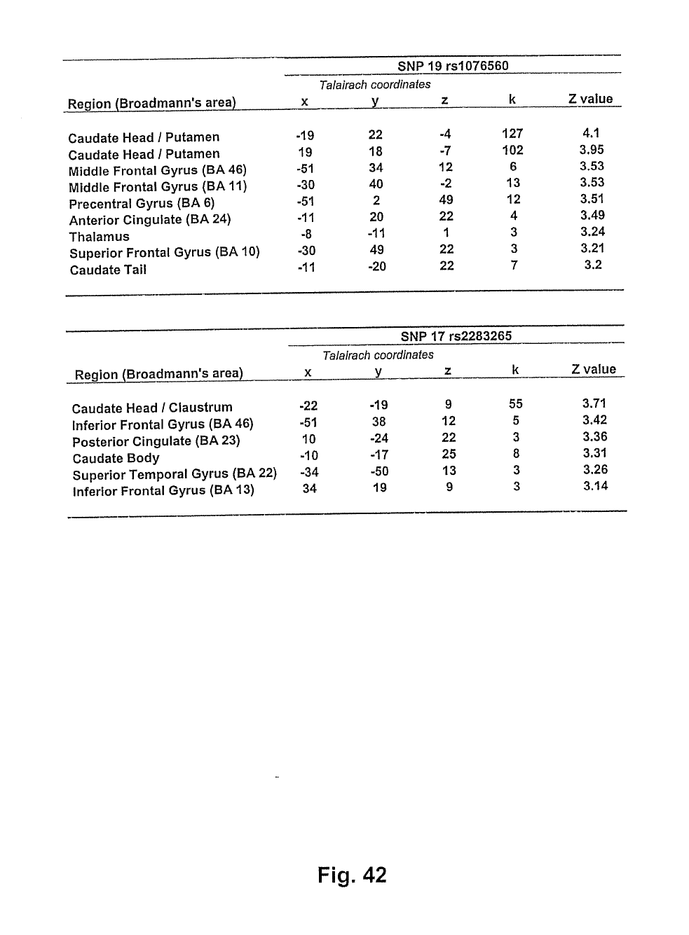

ANOVA also revealed a similar effect of intron 5 SNP17 genotype. As shown for SNP19, the G/T SNP17 genotype was associated with greater activity than GG in several brain regions, including left caudate head and body, left claustrum, left and right inferior frontal gyms, left superior temporal gyms, and right posterior cingulate (FIG. 42). No significant difference was found for the inverse contrast (G/G>G/T). These results suggest that penetrance of these two intronic SNPs on in vivo activity of the working memory network is robust, especially on striatal firing.

Discussion

This study demonstrates the presence of novel regulatory polymorphisms in the gene encoding the dopamine receptor DRD2. Use of allelic expression analysis of human brain autopsy tissue samples, followed by SNP scanning of the DRD2 locus, and in vitro validation using reporter gene and minigene constructs, has revealed one upstream promoter polymorphism and two intronic SNPs affecting DRD2 splicing. Moreover, genotype-driven changes in DRD2 splicing robustly affect activity of the cortical and subcortical working memory network in humans, especially in the striatum, which is rich in dopamine projections and DRD2 receptors.

The promoter SNP2, located 844 bps upstream of the transcription start site, significantly affects allelic mRNA expression of DRD2, supporting a regulatory role in human brain. While both rat and human DRD2 contain a promoter region ˜300 by upstream, sequences further upstream often contain tissue-specific expression or silencer domains. Our reporter gene results reveal a repressor region capable of inhibiting transcription, located −600 to −963 bp's upstream of previously tested promoter region (Arinami, T., et al. (1997) Hum Mol Genet. 6, 577-82) incorporated herein by reference. This repressor region may mask any effects of the proposed promoter polymorphism, SNP4 (−141 Ins/Del). Importantly, the C allele (minor allele) of the new promoter SNP2 confers higher transcriptional activity compared to the main T allele, indicating a disinhibition or gain-of-function for the C allele—potentially a penetrant property even in heterozygotes. Conserved sequences flanking SNP2 contain putative sites for transcription factors, such as E47, ANF, NF-X3, and HSF1, whereas the minor C allele lacks binding sites for ANF and HSF1 but generates a new putative site for AREB6 (TRANSFAC, version 8.3) (Farre, D., et al. (2003) Nucleic Acids Res 31, 3651-3) incorporated herein by reference. Detailed molecular studies are needed to resolve the nature of the regulatory events. We note that SNP2 accounts for only part of the observed AEI ratios.

A second striking finding is the discovery of intronic SNP17 and SNP17 flanking exon 6, linked to DRD2 splicing. Whereas trans regulation can account for splicing differences between prefrontal cortex and striatum, analysis of allelic expression for each splice variant demonstrated an additional role for cis-acting polymorphisms. Both SNP17 and SNP19 modulate distinct splice factor (SRP protein) binding sites, with the G allele generating a putative binding site for SRP55 (SNP17), and the T allele forming SC35 (SNP17) or SRP40 (SNP19) binding sites (ESE finder, Cartegni, L., et al. (2003) Nucleic Acids Res 31, 3568-71) incorporated herein by reference. In vitro experiments using minigenes carrying major or minor alleles for both SNPs confirmed that the minor alleles produced significantly less DRD2S than the major alleles. While SNP17 and SNP19 are tightly linked to each other, minigene constructs with all possible allele combinations indicate that both SNP17 and 19 show activity and may cooperate when present together (FIG. 38). Our results indicate that SNP17 and SNP19 largely affect formation of the S splice variant.

To test the relevance of these novel DRD2 polymorphisms, we measured their effect on brain activity during working memory in normal human subjects. The promoter SNP2 failed to alter activity of the working memory network, as measured with fMRI. Two factors could account for this negative result. First, SNP2 accounts for only a portion of the allelic expression differences observed in our study. Second, SNP2 is expected to affect overall DRD2 density, rather than splice variant L/S ratios, the latter potentially having a stronger effect on neuronal firing. However, it is possible that penetrance of this SNP may be revealed when analyzed in larger cohorts, with different phenotypes.

The fMRI results did reveal robust associations between intronic SNP17 and 19 with activity of the ventral striatum, thalamus, dorsolateral prefrontal cortex, and premotor cortex during working memory. Heterozygote subjects had greater activity in these brain regions despite similar accuracy and reaction time, suggesting that the effects do not result from behavioral differences. Homozygous subjects for the minor variant were not observed in the present study.

These results are consistent with the known role of dopamine in modulating the cortico-striato-thalamo-cortical network. An important part of this modulation is mediated via striatal DRD2 receptors. Dopamine decreases both GABA and glutamate inputs to striatal spiny neurons by binding to DRD2 receptors. While the overall number of DRD2 receptors (both variants) modulates GABA mediated inhibition of striatal neurons, the DRD2 receptor-dependent inhibition of glutamate release preferentially involves the DRD2S variant. Thus, reduced DRD2S expression is expected to increase excitability of striatal medium spiny neurons. Consistent with these electrophysiology experiments in rodents, our results demonstrate that the minor alleles of the intronic SNP17/19 associated with low DRD2 expression are also associated with greater activity in human striatum during working memory. Our results also demonstrate greater activity in other regions of the working memory network, including the dorsolateral prefrontal cortex, parietal cortex, and thalamus. It is possible that greater striatal activity enhances activity in all other brain regions in the cortico-striato-thalamo-cortical network. In addition, because of the cis-acting splicing effects from intronic SNP17/19, the trend of generating less DRD2S in heterozygotes is likely also present in areas outside the striatum.

Our results indicate that previously suggested DRD2 polymorphisms appear not to contribute directly to mRNA expression and splicing. On the other hand, we show here that the SNP23 Taq1A allele is in rather strong linkage disequilibrium (D′=0.855) with the minor allele of the intronic SNP17/19, for the first time providing a mechanistic basis for the clinical associations observed with Taq1A (SNP23).

The results of the present study demonstrate that use of allelic mRNA expression together with functional brain imaging can reveal frequent regulatory variants that had escaped previous genetic analysis even in intensely studied genes such as DRD2. This approach facilitates clinical association studies as only the functional polymorphisms are tested, rather than marker SNPs in partial linkage disequilibrium with them, a valuable complement to the emerging genome-wide association studies that have generated numerous candidate genes for many disorders. As there was no prior demonstration of genetic factors in DRD2 splicing, we also emphasize that the power of combining novel genetic techniques with functional imaging of the human brain allows in vivo demonstration of specific neurobiological mechanisms modulating neuronal activity during specific cognitive tasks.

Material and Methods

Postmortem Human Brain Tissues

105 DNA and RNA samples, extracted from prefrontal cortex autopsy tissues, were obtained from The Stanley Medical Research Institute's brain collection, courtesy of Drs. Michael B. Knable, E. Fuller Torrey, Maree J. Webster, and Robert H. Yolken (Chevy Chase, Md.). Tissues characteristics were: average post-mortem interval (PMI) 33 hrs, age 19 to 64 years, with 102 Caucasians, 1 African American, 1 Hispanic, and 1 Native American. In addition, 25 frozen striatum tissues from Caucasians (18-53 years old, PMI less than 16 hrs) were from the Brain and Tissue Bank for Developmental Disorders at the University of Maryland (Baltimore, Md.). DNA and RNA were extracted as described in Zhang, Y., et al. (2005) J Biol Chem 280, 32618-24, incorporated herein by reference. cDNA was synthesized with reverse transcriptase II (Invitrogen, Carlsbad, Calif.) using both gene specific primers and oligo(dT).

Genotyping with GC-Clamp Assay, Allele-Specific PCR, and SNPlex

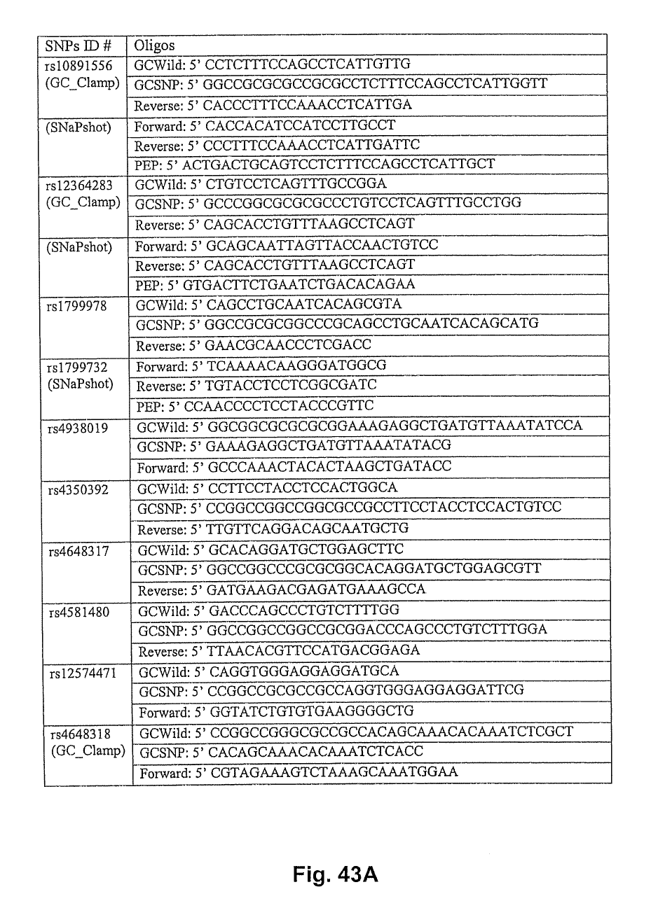

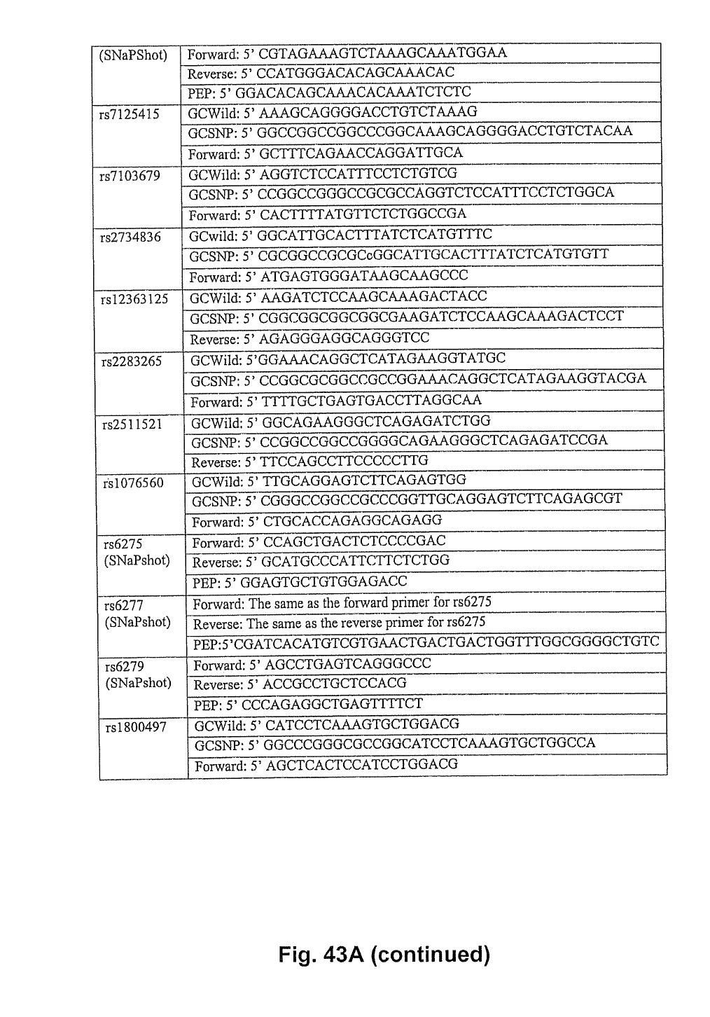

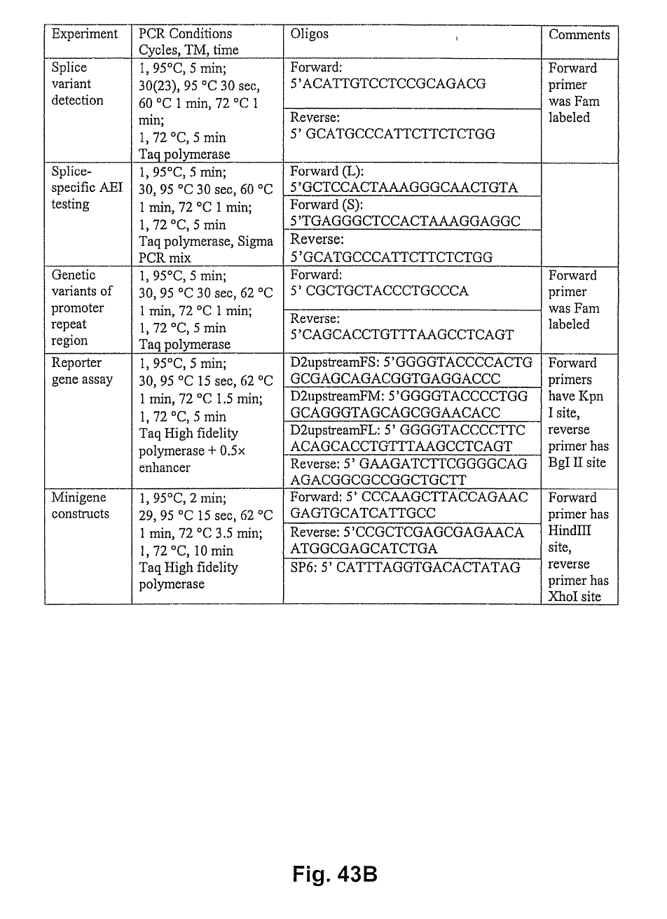

Spanning the DRD2 locus, 23 SNPs were genotyped in 105 prefrontal cortex samples. SNP2, SNP17, and SNP19 were genotyped for DNA samples (n=100) from the University of Bari (Bari, Italy). SNPs were analyzed with allele-specific PCR primers as described in Papp, A. C., et al. (2003) Biotechniques 34, 1068-72, incorporated herein by reference, or SNaPshot (Applied Biosciences (ABI), Foster City Calif.) (Zhang, Y., et al. (2005) J Biol Chem 280, 32618-24). SNP13 and SNP15 were also genotyped with SNPlex (ABI). PCR and SNaPshot primers are shown in the FIG. 42A. SNP21 and SN P22 were genotyped with SNPlex and SNaPshot, and SNP20 with allel-specific primers and SNaPshot, yielding identical results for all samples analyzed by both method (n=105). All SNPs were in Hardy-Weinberg equilibrium in the prefrontal cortex samples except SNP23 (p=0.03). The GAA/GAAA variable repeat region was analyzed using fluorescently labeled PCR primers (FIG. 43B) on an ABI 3730 sequencer.

DRD2 mRNA Levels by Real-Time RT-PCR

RT-PCR was performed with β-actin as an internal control, using 50 ng cDNA per sample, 200 nM primers (as used for SNP20, supporting Table), SYBR-Green, and AmpliTaq Gold and AmpErase UNG), on an ABI 7000 (Pinsonneault, J. K., Papp, A. C. & Sadee, W. (2006) Hum Mol Genet. 15, 2636-49) incorporated herein by reference. Cycle thresholds of DRD2 and β-actin were compared to determine relative mRNA DRD2 expression.

Quantitative Detection of Splice Isoforms by Fluorescence-PCR

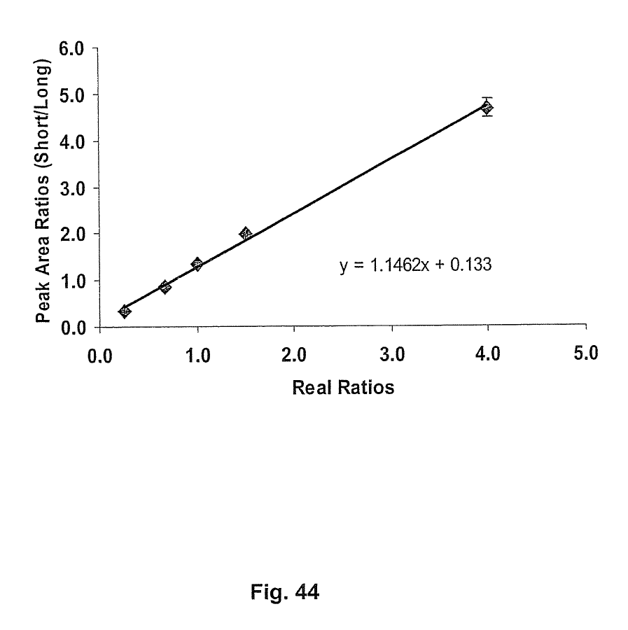

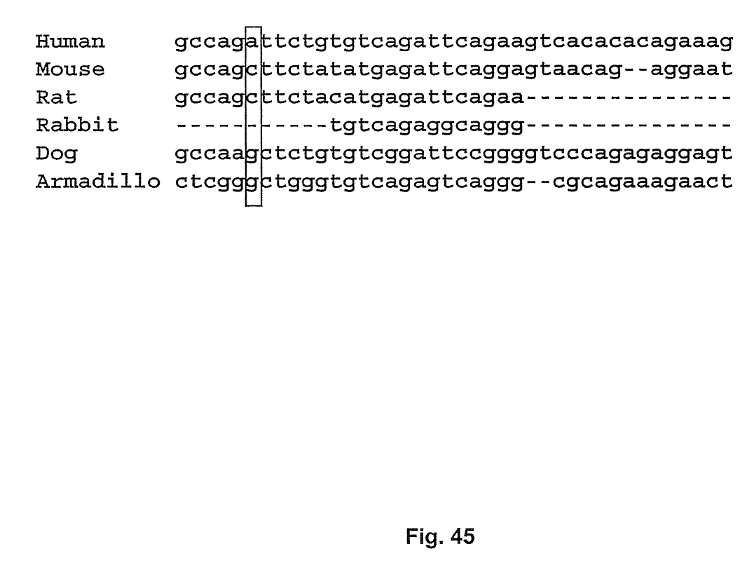

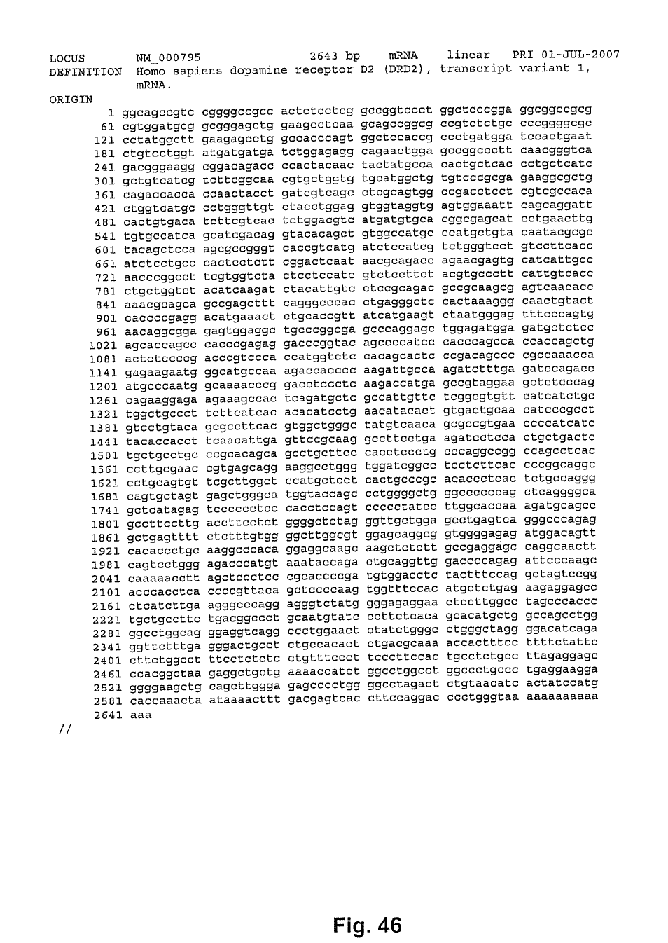

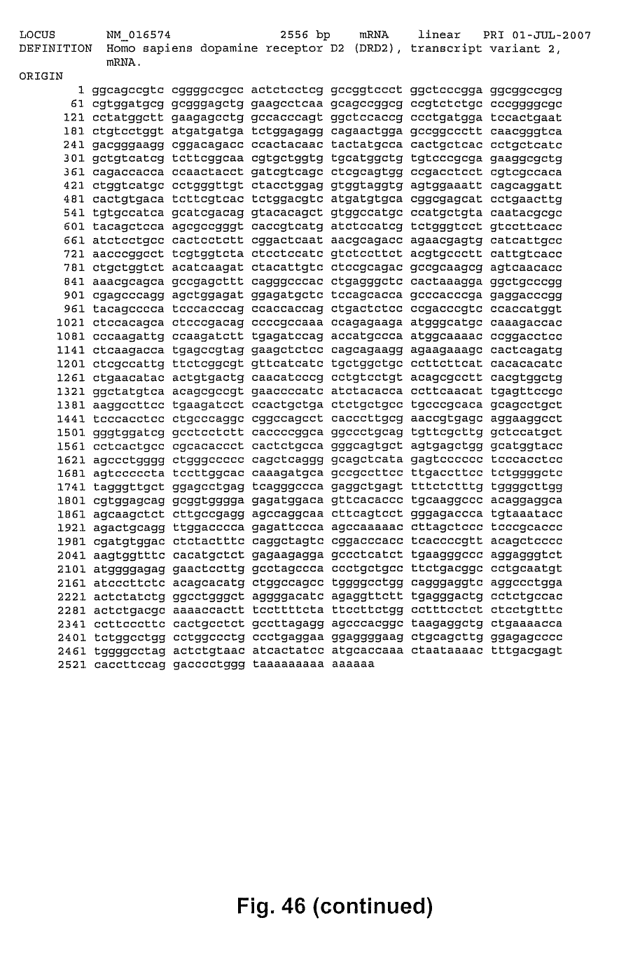

DRD2L and DRD2S, were measured after PCR amplification using a Fam-labeled exon 5 forward primer and an exon 7 reverse primer FIG. 42B on an ABI 3730 (ABI), as described for CACNA1C (Wang, D., Papp et al. (2006) Pharmacogenet Genomics 16, 735-45) incorporated herein by reference. Standard curves were constructed using varying mixtures of cloned DRD2L and S cDNA (DRD2L from UMR cDNA Resource Center, Rolla, Mo.) (FIG. 44 and Supporting Table).

Allele-Specific DRD2 mRNA Expression (SNaPshot, ABI)

SNaPshot is a PCR/primer extension method (SNaPshot, Applied Biosciences). Three marker SNPs located in transcribed regions (SNP20 and SNP21 in exon 7, and SNP22 in 3′UTR) were used for measuring allelic ratios of genomic DNA and mRNA (after conversion to cDNA). In brief, a ˜100 by fragments of DNA or cDNA flanking the polymorphic site were PCR-amplified and SNaPshot reactions performed using extension primers (FIG. 42A). Reaction products were analyzed on an ABI 3730, using Gene Map software (ABI). Relative amounts of the two alleles in genomic DNA and cDNA were determined from peak area ratios, and allelic cDNA ratios normalized to allelic ratios of genomic DNA, which varied within a narrow range (e.g., 0.96±0.05 for SNP21). For assay validation, normalized allelic mRNA ratios where compared where two marker SNPs were heterozygous in the same individual.

To determine allelic expression for DRD2S and DRD2L, each splice variant was separately amplified using specific primers (FIG. 42B), and allelic mRNA expression ratios were measured for each splice variants with SNaPshot analysis using SNP21 and SNP20. Significant differences in allelic mRNA ratios between DRD2L and S reveal the presence of cis-acting factors in splicing.

DRD2 Promoter Constructs for Reporter Gene Assays

Promoter fragments were amplified from genomic DNA of two subjects heterozygous for SNP2 but homozygous for all other SNPs within the amplified regions (FIG. 42B), cloned into PGL3_basic vector upstream of luciferase gene using Kpn I and Bgl II cloning sites (Promega Biosciences, CA). The constructs were tested for regulatory activity of SNP2 in HEK-293 cells and SH-SY5Y cells. The three DNA fragments were (from short to long) Pro_S_(−283 to +292 as used by Arinami, T., et al. (1997) Hum Mol Genet. 6, 577-82), Pro_M, −600 to +292, and Pro_L, −963 to +292; transcription start site +1 (Gandelman, K. Y., et al. (1991) J Neurochem 56, 1024-9). Pro_L constructs contain a C or T allele of SNP2 (Pro_LC and Pro_LT), and a polymorphic GAA/GAAA repeat region (−806˜−629). Pro_L was amplified from genomic DNA of two subjects homozygous for 8 and 4 nucleotide deletions (repeat variants 360 (Pro_L1) and 364 (Pro_L2); FIG. 33) compared to reference variant 368. This resulted in four constructs: Pro_LT1, Pro_LC, Pro_LT2 and Pro_LC2 (FIG. 34A).

Cell Culture and Promoter Activity

Human embryonic kidney cells (HEK-293) and SH-SY5Y were cultured in DMEM/F12 media containing 10% fetal bovine serum, penicillin (10 units/ml), and streptomycin (10 μg/ml), at 37° C. with 5% CO₂. 24-hours before transfection, 1-2×10⁵ cells were planted into 24-well plates, and transient transfection was performed with FuGENE HD Transfection Reagent (Roche Applied Science, Indianapolis, Ind.) in serum free medium for 5 hours. As a transfection control, renilla luciferase constructs were cotransfected with PGL3 fused constructs at a 1:20 ratio. Cells were harvested after 48 hours and transferred to 96-well plates, and luciferase activity was detected with Dual-Glo luciferase assays (Promega, Madison, Wis.) on a fluorescence plate reader (PerkinElmer, Shelton, Conn.). Three independent transfections and duplicate luciferase assays were performed for each construct and cell line.

Alternative Splicing Using a DRD2 Minigene

Two DRD2 minigenes consisting of exons 5-7 and introns 5 and 6 were amplified from genomic DNA carrying G-G and T-T alleles of intronic SNP17 and SNP19. G-T and T-G haplotypes were generated with use of a restriction enzyme located between SNP17 and SNP19. The constructs were inserted downstream of the T7 promoter of pcDNA 3 (Invitrogen, CA) and sequenced, confirming the intended haplotypes, plus two additional SNPs (SNP16 and SNP21) (FIG. 38) not associated with splicing (FIG. 36A). Minigene constructs were transfected into HEK-293 cells as described above, and RNA was isolated after 45 hours with Trizol (Invitrogen, CA). For cDNA synthesis, a plasmid-specific primer SP6 (5′ CATTTAGGTGACACTATAG 3′) was used to avoid synthesis of endogenous DRD2 cDNA. Splice variants were assayed by PCR using fluorescently labeled primers (FIG. 42B).

Statistical Analysis of Molecular Genetics Results

Linkage disequilibrium between SNPs (expressed as D′) and haplotypes were calculated using HelixTree™ (Golden Helix, Inc., Bozeman, Mont.). The presence of allelic mRNA expression imbalance (AEI) was determined with normalized cDNA ratios (peak area ratios of cDNA/mean of the peak area ratios of DNA), using Student's t-test to assess deviation from unity in the mRNA ratios, with a minimum allelic mRNA expression ratio of ˜1.2 or 1/1.2 (˜20%) considered distinct from genomic DNA ratios (presence of AEI). Association between genotype status (heterozygous or homozygous) with AEI was tested with Fisher's Exact tests, and controlling for false discovery rates (Benjamini, Y., Hochberg, Y. (1995) Journal of Royal Statistical Society B57, 289-300). To test for epistasis, 2-loci adjusted P values were calculated for the combination of any two SNPs using HelixTree™. To determine whether allelic expression differed between splice variants, we used a threshold of 1.25-fold difference in allelic mRNA ratios between DRD2L and DRD2S. The presence or absence of allelic splice differences then served as phenotype for SNP scanning.

Working Memory in Normal Human Subjects and Genotype Association

All subjects were Caucasians and were genotyped for SNP2, SNP17, and SNP19. Each SNP displayed Hardy-Weinberg equilibrium. To examine the effect of genotypes on working memory associated brain activity independent of sample size, demographic or behavioral variation, we selected subjects to control for these variables (FIG. 39), divided equally into matched subjects homozygous for the main allele of each SNP and those heterozygous for the minor allele (homozygotes were not observed in the study). Subject numbers were 44 (SNP19), and 34 (SNP2 and SNP17). All 17 subjects heterozygous for SNP17 were also heterozygous for SNP19, while 5 subjects were heterozygous only for SNP19.

All subjects had normal or corrected-to-normal visual acuity. Exclusion criteria were presence of neurological or psychiatric disorders, any pharmacological treatment or medical condition that might have influenced cerebral metabolism or blood flow, drug abuse, and past head trauma with loss of consciousness. All subjects gave written informed consent after the procedure was fully explained to them. The protocol was approved by the local IRB (Comitato Etico Locale Indipendente Azienda Ospedaliera “Ospedale Policlinico Consorziale” Bari).

Working Memory Task

During fMRI, all subjects completed a blocked paradigm of the N-Back task (Bertolino, A., et al. (2006) J Neurosci 26, 3918-22; Bertolino, A., et al. (2004) Am J Psychiatry 161, 1798-1805) each incorporated herein by reference. Briefly, “N-back” refers to how far back in the sequence of stimuli the subject had to recall. The stimuli consisted of numbers (1-4) shown in random sequence and displayed at the points of a diamond-shaped box. There was a visually paced motor task, which also served as a non-memory guided control condition (0-Back) that presented the same stimuli, but simply required subjects to identify the stimulus currently seen. In the working memory condition, the task required the recollection of a stimulus seen two stimuli (2-Back) previously while continuing to encode additionally incoming stimuli. Performance data were recorded as the number of correct responses (accuracy) and as reaction time.

Acquisition of fMRI Data

Each subject was scanned using a GE Signa 3T scanner with a standard head-coil (Milwaukee, Wis.). Echo planar imaging BOLD fMRI data were acquired as described previously (TE=30 msec, TR=2 seconds, 20 contiguous slices, voxel dimensions=3.75×3.75×5 mm) (Bertolino, A., et al. (2004) Am J Psychiatry 161, 1798-1805) incorporated herein by reference. We used a simple block design in which each block consisted of eight alternating 0-Back and 2-Back conditions (each lasting 30 seconds), obtained in 4 min and 8 sec, 120 whole-brain scans. The first four scans were acquired to allow the signal to reach steady state and were not included in the final analysis.

Demographic and Behavioral Data Analysis

One-way ANOVAs and chi² were used to evaluate the effects of genotype on demographics as well as on behavioral performance at the N-back (accuracy and response time).

fMRI Data Analysis:

Data analysis was performed using SPM2 (available online at www.fillon.ucl.ac.uk/spm/software/spm2). All fMRI data were reconstructed, registered, linear detrended, globally normalized, and then smoothed (10 mm Gaussian kernel) before analysis within SPM2. fMRI data were analyzed as a time series modeled by a sine wave shifted by an estimate of the hemodynamic response. Individual subject maps were created using t statistics (2-Back>0-Back). These individual contrast images were then used in second-level random effects models to determine task-specific regional responses at the group-level with one-sample t-tests (main effects of task). To remove anatomical areas that were not activated in the main task effect, we restricted the second level random effects analysis to areas that were activated during the task. A functional mask was created by using the activation maps from 2-Back>0-Back contrasts (p<0.05, k=3) limiting the analysis to the working memory cortical and subcortical network. This procedure controls for the possibility that potential differences between the groups arise from areas that are engaged by only one of the groups. Using this mask, separate ANOVAs with genotype as a grouping factor was performed on 2-back>0-back contrasts. Because of our strong a priori hypothesis on differential response of striatal regions and working memory cortical network, and use of a rigorous random effects statistical model, we chose a statistical threshold of p<0.001, k=3. Since areas within the working memory cortical network represented a priori regions of interest, we corrected for multiple comparisons the statistical threshold with a Family Wise Error small volume correction (using a 10 mm radius sphere centered on prefrontal and striatal coordinates, p=0.01). Statistically significant group differences were reported as voxel-intensity z values. For anatomical localization, statistical maxima of activation were converted to conform to the standard space of Talairach and Tournoux (Talairach, J. & Tournoux, P. (1998) Co-planar stereotaxic atlas of the human brain, Thieme Medical Publishers New York, N.Y.).

### Example 4

### Clinical Association of the Functional Polymorphisms

TPH2 and MAOA are both members of the serotonin pathway, critically involved in its biosynthesis and degradation. TPH2 is the rate limiting step of serotonin synthesis, while MAOA is the enzyme that catalyzes the oxidation of biogenic amines, including serotonin. We have identified biomarkers that detect an increase of TPH2 and a decrease of MAOA activity. If these alleles—a gain of function in TPH2 and a loss of function in MAOA—cooperate when in combination, a significant increase in serotonin levels could result, impacting various disease risks. DRD2 plays a key role in dopamine neurotransmission, central to the pathophysiology of schizophrenia, Parkinsonism, addiction, and a number of other mental disorders. As well as the clinical associations disclosed for the functional SNPs in these genes, we expect that combined application of the SNPs in these genes will reveal penetrant effects in some of the main CNS disorders.

We measured some of the listed polymorphisms in various clinical cohorts. Brain tissue autopsies were obtained for 105 subjects from the Stanley Foundation, formerly diagnosed with schizophrenia, bipolar disorder, and controls, with further clinical information (e.g., suicide, depression) (described above). 80 liver autopsy samples were received from the Stanley Foundation of additional subjects diagnosed with schizophrenia, bipolar disorder, major depression and controls.

In addition, peripheral blood lymphocytes were obtained for a sample of pre- and post-partum women with depression (the “TPPD cohort”), with some being treated with antidepressants. The TPPD cohort consisted of 160 specimen samples belonging to volunteer maternity and postpartum patients from the Women's Health Concerns Clinic, St Joseph's Healthcare, in Ontario, CA (collaboration with Dr. Meir Steiner). All participants have been clinically characterized and diagnosed with the following: post partum depression (36), major depression (51), adjustment disorder (32), bipolar disorder (8), general anxiety disorder (12) or no disorder (control group 21). 18 additional female controls free from depression or bipolar disorder were obtained from the Stanley Foundation brain collection. Of the 160 subjects, 87 were being treated for depression with the following antidepressants: Celexa (20), Cipralex (7), Effexor (19), Paxil (15), Prozac (7) and Zoloft (19). All but Effexor are selective serotonin reuptake inhibitors (SSRIs). Effexor is a serotonin/norepinephrine reuptake inhibitor (SNRI). 19.5% (17 patients) showed no response to the treatment. Drug response was assessed with standard tests.

Association of DRD2 Promoter SNP rs12364283 with Schizophrenia (Stanley Cohort)

In a first test to assess the clinical penetrance and the role of rs12364283, we performed an association analysis using the 105 samples from the Stanley DNA collections (35 schizophrenic, 35 bipolar, and 35 control subjects). This revealed a significant association of rs12364283 with schizophrenia, with minor allele carriers having higher frequency in the patient population (χ²=6.89, P=0.009 (n=95), odds ratio (AA/AG)=0.442, 95% confidence interval, 0.274-0.712).

Association of MAOA Polymorphisms with Bipolar Disorder and Suicide (Stanley Cohort)

We genotyped rs1801291 (C and T alleles) and the pVNTR (3 and 4 repeat) previously shown to have significant clinical associations. Either polymorphism alone did not show strong associations, while the haplotype formed by these two variants gave significant associations with bipolar disorder and suicide in females.

TABLE 9
MAOA pVNTR/rs1801291 2-loci haplotypes in males and females with
bipolar disorder and suicide in females.

| | | | | | | | Females | | |
|---|---|---|---|---|---|---|---|---|---|
| | Female | Female | | Male | Male | | Not | Females | |
| | bipolar | control | | bipolar | control | | Suicide | Suicide | |
| | n = 18 | n = 9 | pValue | n = 17 | n = 26 | pValue | n = 25 | n = 11 | pValue |
| 4, C | 0.62 | 0.61 | 1 | 0.65 | 0.65 | 1 | 0.64 | 0.45 | 0.1953 |
| 3, T | 0.12 | 0.39 | 0.03 | 0.18 | 0.19 | 1 | 0.28 | 0.23 | 0.7751 |
| 4, T | 0.07 | 0 | 0.54 | 0 | 0 | 1 | 0.06 | 0.00 | 0.5481 |
| 3, C | 0.18 | 0.00 | 0.08 | 0.12 | 0.08 | 0.71 | 0.02 | 0.32 | 0.0007 |
| 5, C | 0 | 0 | 1 | 0.06 | 0 | 0.15 | 0 | 0 | 1 |
| 5, T | 0 | 0 | 1 | 0 | 0.08 | 0.15 | 0 | 0 | 1 |

Association of MAOA, DRD2, and TPH2 Polymorphisms on Treatment Outcome with Antidepressants in Pre- and Post-Partum Females (TPPD and Stanley Cohort)

We measured the genotype of the TPPD and Stanley cohort samples and associated these with clinical outcomes. Some of the genotyping had been performed before the functional polymorphisms had been identified in DRD2, and we show here results with SNPs that are closely linked to the functional SNPs. This may have reduced somewhat the significance of any associations if linkage was incomplete. Nevertheless, for each gene several significant associations can be observed (Table 10). In Table 10, columns are as follows: Drug response is defined as non-responders compared to responders. TPPD post partum depression compared to controls, TPPD major depression compared to controls and Stanley depression compared to controls. The listed MAOA SNPs are in partial linkage disequilibrium with the pVNTR. DRD2 SNPs rs1125394 and rs2075654 are strongly linked to the functional SNPs rs2283265 and rs1076560 identified in Example 3. Therefore the results obtained with SNPs rs1125394 and rs2075654 are reflective of results expected from SNPs rs2283265 and rs1076560. The proposed functional TPH2 SNP (rs7305115) is in tight linkage with rs6582078, and therefore both yielded similar results in this cohort.

TABLE 10
Genotype associations with depression and drug response.

| | TPPD | TPPD Samples | TPPD Samples | Stanley |
|---|---|---|---|---|
| | Samples Drug | Post Partum | Major | Samples |
| | response | Depression | Depression | Depression |
| Marker | Adjusted P | Adjusted P | Adjusted P | Adjusted P |
| MAOA pVNTR | 0.024 | 0.356 | 0.245 | 0.070 |
| MAOA rs6323 Exon 8 | 0.026 | 0.138 | 0.038 | 0.790 |
| MAOA rs979606 | 0.126 | 0.126 | 0.045 | 0.479 |
| MAOA rs979605 | 0.126 | 0.228 | 0.036 | 0.591 |
| MAOA rs1801291 | 0.182 | 0.171 | 0.103 | 0.790 |
| Exon14 | | | | |
| MAOA rs3027407 | 0.189 | 0.232 | 0.059 | 0.790 |
| DRD2 rs1125394 | 0.001 | 0.049 | 0.425 | 0.005 |
| 5′UTR | | | | |
| DRD2 rs2075654 | 0.001 | 0.032 | 0.337 | 0.002 |
| Intron2 | | | | |
| TPH2 rs6582078 | 0.954 | 0.798 | 0.440 | 0.049 |
| TPH2 rs7305115 | 0.962 | 0.802 | 0.817 | 0.049 |

Since both the pVNTR and 3′-SNPs in MAOA gave significant associations (even though they are only in partial linkage disequilibrium), we tested whether both together yield stronger associations. This was indeed the case in association studies for antidepressant treatment outcome. The same haplotype associated with suicide in females (Table 9) is also associated with poor drug response (Table 10).

MAOA 2-Loci Haplotypes and Antidepressant Drug Response in Females (TPPD Cohort).

The MAOA locus is located on the X chromosome and spreads over 90.6 kilobases in a region of high linkage disequilibrium. One MAOA haplotype, defined by a promoter variable nucleotide tandem repeat (pVNTR) combined with a synonymous SNP in the 3′ end of the gene, is significantly associated with non-response to antidepressants (Table 11).

TABLE 11

| MAOA pVNTR/ | Responder | Non-responder | |
|---|---|---|---|
| rs1801291 Haplotype | n = 140 | n = 34 | 2 tailed p Value |
| 4, C | 57% | 51% | 0.57 |
| 3, T | 30% | 23% | 0.53 |
| 3, C | 5% | 16% | 0.03 |
| 4, T | 5% | 3% | 1.00 |
| 3.5, C | 1% | 6% | 0.17 |

We project that combining these genes with other known factors can also result in very strong associations. For example, with respect to antidepressant drug response, there is a strong 2 loci interaction of TPH2 with the SERT LPR (a commonly genotyped promoter variant in the serotonin transporter gene) (data not shown). SERT LPR/TPH2 rs7305115 P: 1.08E-12 Adjusted P: 3.06E-09 Bonferroni P: 4.88E-06. This strong association has promise for having predictive value in deciding drug therapies.

Association Between MAOA, TPH2 and DRD2 Functional Polymorphisms and Substance Abuse, Autism and Schizophrenia

Genes involved in substance abuse include neurotransmitter receptors, transporters and those involved in neurotransmitter metabolism. MAOA is implicated in early drug abuse, and the minor variant is associated with aggressive and risky behavior. The A1 allele of the Taq I polymorphism of the DRD2 gene has been earlier reported to occur in 69% of alcoholics, compared with 20% of controls. Therefore, we project that the DRD2 functional polymorphisms of the present disclosure are associated with alcoholism and other addictive behaviors. Notably, expression of the DRD2 receptor appears to vary between individuals and alternative splicing of DRD2 (S and L forms) is modulated by alcohol intake, yielding different functions.

The newly defined polymorphisms in MAOA, DRD2, and TPH2 each alters the functional expression of the respective gene. Thus, the minor allele of rs7305115 in TPH2 was associated with enhanced expression, while the MAOA SNPs in the 3′-haplotype (possibly together with the pVNTR) associate with reduced expression. Both together are expected to have synergistic effects on serotonin levels in the brain, consistent with the observed associations with clinical phenotypes. However, MAOA also catabolizes dopamine and norepinephrine, so that the in vivo effects can vary according to the context of the disorder. The promoter SNP of DRD2, rs12364283, enhances mRNA expression, with varying in vivo outcomes. On the other hand the minor alleles of intronic SNPs rs2283265 and rs1076560 (in partial linkage disequilibrium with Taq1A1) lower the formation of DRD2S (an inhibitory form) relative to DRD2L (facilitating dopamine transmission). The expected result can be enhanced dopaminergic neurotransmission and added risk in schizophrenia, and other diseases associated with enhanced dopamine activity (e.g. substance abuse).

Attention deficit hyperactivity disorder (ADHD) is a childhood disorder that consists of two distinct underlying types: inattentive and hyperactive (which are moderately correlated), and the combined type. Pharmacological treatment of ADHD is often by stimulants. Stimulant drugs used to treat ADHD bind to the dopamine transporter, inhibiting dopamine reuptake and thus increasing its concentration in the synapse. TPH2 along with 17 other genes including MAOA has been found to be associated with the combined type of ADHD. DRD2 has also been implicated in ADHD. TaqI A of the DRD2 gene is involved in the pathogenesis of childhood ADHD in males. Therefore, the functional polymorphisms in linkage disequilibrium with Taq1A are likely also to be involved in ADHD. However, since Taq1A itself is not a functional polymorphism, intronic SNPs rs2283265 and rs1076560 are expected to have more significant associations.

Autism is a neurodevelopmental disorder that has a high genetic component. Many genes seem to play a role in autism, in particular those involved in neurodevelopment. The serotonin and dopamine pathways have been proposed to be involved in the etiology of autism spectrum disorders. The pVNTR of MAOA has been associated with autism severity. Therefore, we predict that our functional MAOA polymorphisms in the 3′ region are also associated with autism severity, and possibly more tightly linked. Indeed, we project that a haplotype including the above identifies functional polymorphisms together with pVNTR will show the strongest predictive value. Variants in the TPH2 gene have been reported to be associated with autism susceptibility, but our results indicate that any association would be strongest with rs7305115, or SNPs in tight linkage with it.

1. A method for predicting a subject's risk for having or developing a mental disorder comprising detecting the allelic status of one or more polymorphisms in a nucleic acid sample of the subject, wherein the polymorphism is selected from the group consisting of:
i. tryptophan hydroxylase 2 (TPH2)-associated SNP's rs7305115, rs2171363, rs4760815, rs6582078, rs9325202, and combinations thereof; and
ii. a SNP in linkage disequilibrium with one or more SNPs listed in i;
wherein the allelic status of the polymorphism in the subject is predictive of the subject's risk for having or developing a mental disorder.

2. The method of claim 1, further comprising the step of correlating the allelic status of the polymorphism in the subject with the allelic status of the polymorphism in a reference population to predict the subject's risk for having or developing the mental disorder.

3. The method of claim 1, further comprising the step of correlating the allelic status of the polymorphism in the subject with the allelic status of the polymorphism in a reference population to predict whether the subject has a more or less severe phenotype of the mental disorder.

4. The method of claim 1, wherein the mental disorder comprises one or more of the following: substance abuse, attention deficit disorder (ADD), attention deficit hyperactivity disorder (ADHD), anxiety, depression, bipolar disorder, suicidal behavior, behavioral disorder, schizophrenia, Parkinson's disease or autism.

5. The method of claim 1, wherein the mental disorder is one in which serotonin plays a role.

6. (canceled)

7. (canceled)

8. (canceled)

9. (canceled)

10. (canceled)

11. (canceled)

12. (canceled)

13. The method of claim 1, wherein the polymorphism comprises a TPH2-associated rs7305115 or a SNP in linkage disequilibrium with rs7305115 and wherein the presence of a minor allele of the polymorphism is predictive of higher levels of serotonin in a target tissue associated with the mental disorder.

14. The method of claim 1, wherein the polymorphism comprises a TPH2-associated rs7305115 or a SNP in linkage disequilibrium with rs7305115 and wherein the presence of a minor allele of the polymorphism is predictive of a decreased risk for depression or suicidal behavior or both.

15. The method of claim 1, wherein the polymorphism comprises a TPH2-associated haplotype comprising rs7305115 in combination with one or more SNP's rs2171363, rs4760815, rs6582078, rs9325202, and wherein the presence of a minor allele of the polymorphism is predictive of a reduced risk for depression or suicidal behavior or both.

16. The method of claim 1, wherein the polymorphism comprises a TPH2-associated 5 SNP haplotype TAAGA comprising minor alleles of rs2171363, rs4760815, rs7305115, rs6582078, and rs9325202, wherein the presence of the polymorphism is predictive of high levels of TPH2 mRNA expression in the brain.

17. The method of claim 1, wherein the polymorphism comprises a TPH2-associated 5 SNP haplotype TAAGA comprising minor alleles of rs2171363, rs4760815, rs7305115, rs6582078, and rs9325202, wherein the presence of the polymorphism is predictive of a reduced risk for depression or suicidal behavior or both.

18. The method of claim 1, further comprising detecting the allelic status of one or more polymorphisms selected from the group consisting of:
iii. MAOA-associated SNPs rs6323, rs2205718, rs979606, rs979605, rs1801291, rs3027407;
iv. a SNP in linkage disequilibrium with SNPs rs6323, rs2205718, rs979606, rs979605, rs1801291, rs3027407;
wherein the presence of a minor allele of at least one TPH2-associated polymorphism in i or ii together with at least one MAOA-associated polymorphism in iii. or iv. is predictive of a higher level of serotonin in a target tissue associated with the mental disorder.

19. The method of claim 18, wherein the presence of said minor alleles of the TPH2-associated or MAOA-associated polymorphisms is predictive of a decreased risk for depression or suicidal behavior or both.

20. (canceled)

21. (canceled)

22. (canceled)

23. (canceled)

24. (canceled)

25. (canceled)

26. (canceled)

27. (canceled)

28. A method of screening a subject for a prognostic biomarker of a mental disorder, comprising detecting the allelic status of one or more polymorphisms in a nucleic acid sample of the subject, wherein the polymorphism is selected from the group consisting of
i. tryptophan hydroxylase 2 (TPH2)-associated SNP's rs2171363, rs4760815, rs7305115, rs6582078, rs9325202, and combinations thereof; and
ii. a SNP in linkage disequilibrium with one or more SNPs listed in i;
wherein the allelic status of the polymorphism in the subject is predictive of the prognostic outcome of the mental disorder.

29. The method of claim 28, further comprising the step of correlating the allelic status of the polymorphism in the subject with the allelic status of the polymorphism in a reference population to predict the prognostic outcome of the mental disorder in the subject.

30. The method of claim 28, further comprising the step of correlating the allelic status of the polymorphism in the subject with the allelic status of the polymorphism in a reference population to predict whether the subject has a more or less severe phenotype of the mental disorder.

31. The method of claim 28, further comprising the step of correlating the allelic status of the polymorphism in the subject with the allelic status of the polymorphism in a reference population to predict the subject's response to treatment.

32. The method of claim 28, wherein the mental disorder comprises one or more of the following: substance abuse, attention deficit disorder (ADD), attention deficit hyperactivity disorder (ADHD), anxiety, depression, bipolar disorder, suicidal behavior, schizophrenia, Parkinson's disease or autism.

33. The method of claim 28, wherein the mental disorder is one in which serotonin plays a role.

34. (canceled)

35. (canceled)

36. (canceled)

37. (canceled)

38. (canceled)

39. The method of claim 28, wherein the polymorphism comprises the TPH2-associated rs7305115 or a SNP in linkage disequilibrium with rs7305115 and wherein the presence of a minor allele of the polymorphism is predictive of an increased resistance to serotonin enhancing drug therapy.

40. The method of claim 28, wherein the polymorphism comprises a TPH2-associated 5 SNP haplotype TAAGA comprising minor alleles of rs2171363, rs4760815, rs7305115, rs6582078, and rs9325202, wherein the presence of the polymorphism is predictive of resistance to serotonin enhancing drug therapy.

41. (canceled)

42. (canceled)

43. A kit comprising an assay for detecting the allelic status of one or more polymorphisms in a nucleic acid sample of a subject, wherein the polymorphism is selected from the group consisting of:
i. tryptophan hydroxylase 2 (TPH2)-associated SNP's rs2171363, rs4760815, rs7305115, rs6582078, rs9325202, and combinations thereof; and
ii. a SNP in linkage disequilibrium with one or more SNP's listed in i.

44. The kit of claim 43, further comprising instructions for correlating the assay results with the subject's risk for having or developing a mental disorder.

45. The kit of claim 43, further comprising instructions for correlating the assay results with the subject's prognostic outcome for the mental disorder.

46. The kit of claim 43, further comprising instructions for correlating the assay results with the probability of success or failure of a particular drug treatment in the subject.

47-56. (canceled)


SEQUENCE LISTING (306 sequences)

SEQ ID NO: 1 - DNA, 12 bp, Artificial Sequence
  Description of Artificial Sequence Synthetic oligonucleotide
gaatctggcaaa

SEQ ID NO: 2 - DNA, 30 bp, Homo sapiens
tccagcagagagaaaccagttaattcagcg

SEQ ID NO: 3 - DNA, 30 bp, Homo sapiens
gctctaaaaataacagaacctaagtgatga

SEQ ID NO: 4 - DNA, 30 bp, Homo sapiens
acaaaagagaaaacaaagctgaaatgctgc

SEQ ID NO: 5 - DNA, 30 bp, Homo sapiens
ggattttgacaactatttctagaatttgca

SEQ ID NO: 6 - DNA, 30 bp, Homo sapiens
aaatggtctcgggaaggtgaccgagaaaga

SEQ ID NO: 7 - DNA, 30 bp, Homo sapiens
aaatttgactgttatttgttgagactatca

SEQ ID NO: 8 - DNA, 30 bp, Homo sapiens
ttctttccttccccacctttggtgtttctg

SEQ ID NO: 9 - DNA, 30 bp, Homo sapiens
cctcttcaaatcatgataatttatataaca

SEQ ID NO: 10 - DNA, 30 bp, Homo sapiens
ccggcatggctcagatcccctctacacccc

SEQ ID NO: 11 - DNA, 30 bp, Homo sapiens
gaacgtataacctgtgtgggagtctggaag

SEQ ID NO: 12 - DNA, 30 bp, Homo sapiens
tattatcattagtctctctgtatccctatc

SEQ ID NO: 13 - DNA, 30 bp, Homo sapiens
attagttaccaactgtcctcagtttgccag

SEQ ID NO: 14 - DNA, 30 bp, Homo sapiens
tacctggaagtcatgtgctttgtatgaaac

SEQ ID NO: 15 - DNA, 30 bp, Homo sapiens
tctttcttctagcacagtaattggcaataa

SEQ ID NO: 16 - DNA, 30 bp, Homo sapiens
ctttttttgctgagtgaccttaggcaagtt

SEQ ID NO: 17 - DNA, 30 bp, Homo sapiens
agccacccatctcactggcccctccctttc

SEQ ID NO: 18 - DNA, 30 bp, Homo sapiens
cttccaatgggagctgtcattaagtgcatg

SEQ ID NO: 19 - DNA, 30 bp, Homo sapiens
taaaagaggccactgtgcaaatcatctcgt

SEQ ID NO: 20 - DNA, 30 bp, Homo sapiens
agtcaataatatcgttgctttaacaaaaag

SEQ ID NO: 21 - DNA, 30 bp, Homo sapiens
tgaactctgcttttccttttaaatttggca

SEQ ID NO: 22 - DNA, 30 bp, Homo sapiens
atctgggtacaagaacctgaatcaaaggac

SEQ ID NO: 23 - DNA, 30 bp, Homo sapiens
acagaaaagaaattagggctctaatttcct

SEQ ID NO: 24 - DNA, 30 bp, Homo sapiens
cttgattgacatttctacctggcggttgga

SEQ ID NO: 25 - DNA, 30 bp, Homo sapiens
cacagaaaacaatatagagtgaaggagtta

SEQ ID NO: 26 - DNA, 30 bp, Homo sapiens
gaaccgtgagtacctacattaaagcccagg

SEQ ID NO: 27 - DNA, 30 bp, Homo sapiens
atgagcagaaatttcatttttgtacaaggc

SEQ ID NO: 28 - DNA, 30 bp, Homo sapiens
tgcctttcttgagcagagagaccatctctt

SEQ ID NO: 29 - DNA, 30 bp, Homo sapiens
ttctgtgtcagattcagaagtcacacacag

SEQ ID NO: 30 - DNA, 30 bp, Homo sapiens
ccttggaatgctgataagtttaattctatt

SEQ ID NO: 31 - DNA, 30 bp, Homo sapiens
tggtcttatgtatctgggagaagataagcg

SEQ ID NO: 32 - DNA, 30 bp, Homo sapiens
cttaccttctatgagcctgtttcctcatct

SEQ ID NO: 33 - DNA, 30 bp, Homo sapiens
ccctctgaagactcctgcaaacaccacagg

SEQ ID NO: 34 - DNA, 40 bp, Artificial Sequence
  Description of Artificial Sequence Synthetic primer
gagagaaaccagttaattcagcggcttccaatgggagctg

SEQ ID NO: 35 - DNA, 40 bp, Artificial Sequence
  Description of Artificial Sequence Synthetic primer
cagctcccattggaagccgctgaattaactggtttctctc

SEQ ID NO: 36 - DNA, 42 bp, Artificial Sequence
  Description of Artificial Sequence Synthetic primer
ccgagaaagatatctgggtacaagaacctgaatcaaaggacg

SEQ ID NO: 37 - DNA, 43 bp, Artificial Sequence
  Description of Artificial Sequence Synthetic primer
cgtcctttgattcaggttcttgtacccagatatctttctcgga

SEQ ID NO: 38 - DNA, 44 bp, Artificial Sequence
  Description of Artificial Sequence Synthetic primer
gactgttatttgttgagactatcaaacagaaaagaaattagggc

SEQ ID NO: 39 - DNA, 44 bp, Artificial Sequence
  Description of Artificial Sequence Synthetic primer
gccctaatttcttttctgtttgatagtctcaacaaataacagtc

SEQ ID NO: 40 - DNA, 22 bp, Artificial Sequence
  Description of Artificial Sequence Synthetic primer
acgagactttctggcaggactg

SEQ ID NO: 41 - DNA, 24 bp, Artificial Sequence
  Description of Artificial Sequence Synthetic primer
ttaattctccaatggaggaaagga

SEQ ID NO: 42 - DNA, 17 bp, Artificial Sequence
  Description of Artificial Sequence Synthetic primer
gatcccctctacacccc

SEQ ID NO: 43 - DNA, 19 bp, Artificial Sequence
  Description of Artificial Sequence Synthetic primer
aaaggagtcctgctccata

SEQ ID NO: 44 - DNA, 26 bp, Artificial Sequence
  Description of Artificial Sequence Synthetic primer
cagcaagagcacaagaggaagagaga

SEQ ID NO: 45 - DNA, 22 bp, Artificial Sequence
  Description of Artificial Sequence Synthetic primer
gtgtggtgggggactgagtgtg

SEQ ID NO: 46 - DNA, 19 bp, Artificial Sequence
  Description of Artificial Sequence Synthetic primer
catttaggtgacactatag

SEQ ID NO: 47 - DNA, 29 bp, Artificial Sequence
  Description of Artificial Sequence Synthetic primer
atcctgcctagaagtactcagtaatttaa

SEQ ID NO: 48 - DNA, 20 bp, Artificial Sequence
  Description of Artificial Sequence Synthetic primer
acagcctgaccgtggagaag

SEQ ID NO: 49 - DNA, 23 bp, Artificial Sequence
  Description of Artificial Sequence Synthetic primer
agattttagcatttccctgcaca

SEQ ID NO: 50 - DNA, 24 bp, Artificial Sequence
  Description of Artificial Sequence Synthetic primer
tgaaggccaggtacagaggaacta

SEQ ID NO: 51 - DNA, 26 bp, Artificial Sequence
  Description of Artificial Sequence Synthetic primer
cagagagaaaccagttaattcagggt

SEQ ID NO: 52 - DNA, 23 bp, Artificial Sequence
  Description of Artificial Sequence Synthetic primer
cacatagctgtcctactcgtagg

SEQ ID NO: 53 - DNA, 29 bp, Artificial Sequence
  Description of Artificial Sequence Synthetic primer
gcggccgcctgcacagtggcctctttgag

SEQ ID NO: 54 - DNA, 24 bp, Artificial Sequence
  Description of Artificial Sequence Synthetic primer
gattcaggttcttgtacccagctg

SEQ ID NO: 55 - DNA, 42 bp, Artificial Sequence
  Description of Artificial Sequence Synthetic primer
ggcggcggcggcaatttgactgttatttgttgagactataag

SEQ ID NO: 56 - DNA, 26 bp, Artificial Sequence
  Description of Artificial Sequence Synthetic primer
cctttagagggttgtatttctgcact

SEQ ID NO: 57 - DNA, 31 bp, Artificial Sequence
  Description of Artificial Sequence Synthetic primer
actaggaatacttattatttctaaaggccca

SEQ ID NO: 58 - DNA, 20 bp, Artificial Sequence
  Description of Artificial Sequence Synthetic primer
aaatggtctcgggaaggtga

SEQ ID NO: 59 - DNA, 22 bp, Artificial Sequence
  Description of Artificial Sequence Synthetic primer
acttcagaccagagcttccagc

SEQ ID NO: 60 - DNA, 18 bp, Artificial Sequence
  Description of Artificial Sequence Synthetic primer
cctggcacccagcacaat

SEQ ID NO: 61 - DNA, 20 bp, Artificial Sequence
  Description of Artificial Sequence Synthetic primer
agaggccgcgagcgcagcac

SEQ ID NO: 62 - DNA, 20 bp, Artificial Sequence
  Description of Artificial Sequence Synthetic primer
acagcctgaccgtggagaag

SEQ ID NO: 63 - DNA, 39 bp, Artificial Sequence
  Description of Artificial Sequence Synthetic primer
ataccgcgccacatagcactagagtcacttctccccgcc

SEQ ID NO: 64 - DNA, 40 bp, Artificial Sequence
  Description of Artificial Sequence Synthetic primer
cgatggcccactacgtgaactagagtcacttctccccgcc

SEQ ID NO: 65 - DNA, 40 bp, Artificial Sequence
  Description of Artificial Sequence Synthetic primer
ccggcccgccgatcctgcctagaagtactcagtaattcac

SEQ ID NO: 66 - DNA, 35 bp, Artificial Sequence
  Description of Artificial Sequence Synthetic primer
cccgccgccccgagattttagcatttccctgcgcg

SEQ ID NO: 67 - DNA, 33 bp, Artificial Sequence
  Description of Artificial Sequence Synthetic primer
cggcggcggtgaaggccaggtacagaggaagtg

SEQ ID NO: 68 - DNA, 39 bp, Artificial Sequence
  Description of Artificial Sequence Synthetic primer
cggcggcggcgggcagagagaaaccagttaattcagagg

SEQ ID NO: 69 - DNA, 35 bp, Artificial Sequence
  Description of Artificial Sequence Synthetic primer
ggccccgccgcccacatagctgtcctactcgtcgc

SEQ ID NO: 70 - DNA, 20 bp, Artificial Sequence
  Description of Artificial Sequence Synthetic primer
tgcacagtggcctctttcat

SEQ ID NO: 71 - DNA, 34 bp, Artificial Sequence
  Description of Artificial Sequence Synthetic primer
cccggcgcgggattcaggttcttgtacccaggta

SEQ ID NO: 72 - DNA, 30 bp, Artificial Sequence
  Description of Artificial Sequence Synthetic primer
aatttgactgttatttgttgagactatgaa

SEQ ID NO: 73 - DNA, 35 bp, Artificial Sequence
  Description of Artificial Sequence Synthetic primer
cggcccgcgcctttagagggttgtatttctgcacg

SEQ ID NO: 74 - DNA, 42 bp, Artificial Sequence
  Description of Artificial Sequence Synthetic primer
gccgcccgccgactaggaatacttattatttctaaaggcgcg

SEQ ID NO: 75 - DNA, 24 bp, Artificial Sequence
  Description of Artificial Sequence Synthetic primer
tttgattcaggttcttgtacccag

SEQ ID NO: 76 - DNA, 22 bp, Artificial Sequence
  Description of Artificial Sequence Synthetic primer
atgcacttaatgacagctccca

SEQ ID NO: 77 - DNA, 20 bp, Artificial Sequence
  Description of Artificial Sequence Synthetic primer
gccgatccacacggagtact

SEQ ID NO: 78 - DNA, 20 bp, Artificial Sequence
  Description of Artificial Sequence Synthetic primer
actccagggccgactgcggc

SEQ ID NO: 79 - DNA, 21 bp, Artificial Sequence
  Description of Artificial Sequence Synthetic primer
cacctccgatcacgactacgt

SEQ ID NO: 80 - DNA, 18 bp, Artificial Sequence
  Description of Artificial Sequence Synthetic primer
ataccgcgccacatagca

SEQ ID NO: 81 - DNA, 19 bp, Artificial Sequence
  Description of Artificial Sequence Synthetic primer
cgatggcccactacgtgaa

SEQ ID NO: 82 - DNA, 23 bp, Artificial Sequence
  Description of Artificial Sequence Synthetic primer
tgaccgatgtttttcccttctct

SEQ ID NO: 83 - DNA, 20 bp, Artificial Sequence
  Description of Artificial Sequence Synthetic primer
gaacggacgctccattcgga

SEQ ID NO: 84 - DNA, 20 bp, Artificial Sequence
  Description of Artificial Sequence Synthetic primer
taggccttggctgtcagtga

SEQ ID NO: 85 - DNA, 23 bp, Artificial Sequence
  Description of Artificial Sequence Synthetic primer
catcctcattcttacctggcatt

SEQ ID NO: 86 - DNA, 22 bp, Artificial Sequence
  Description of Artificial Sequence Synthetic primer
tgcacttaatgacagctcccat

SEQ ID NO: 87 - DNA, 20 bp, Artificial Sequence
  Description of Artificial Sequence Synthetic primer
ttgtggggacaccacttcct

SEQ ID NO: 88 - DNA, 23 bp, Artificial Sequence
  Description of Artificial Sequence Synthetic primer
gcactgattgattaatttggctc

SEQ ID NO: 89 - DNA, 20 bp, Artificial Sequence
  Description of Artificial Sequence Synthetic primer
aaatggtctcgggaaggtga

SEQ ID NO: 90 - DNA, 26 bp, Artificial Sequence
  Description of Artificial Sequence Synthetic primer
cttgctttaaggaaattagagcccta

SEQ ID NO: 91 - DNA, 21 bp, Artificial Sequence
  Description of Artificial Sequence Synthetic primer
tccagggttccttccaattct

SEQ ID NO: 92 - DNA, 23 bp, Artificial Sequence
  Description of Artificial Sequence Synthetic primer
ttacgctttcctacaaaacaggg

SEQ ID NO: 93 - DNA, 19 bp, Artificial Sequence
  Description of Artificial Sequence Synthetic primer
ggaaggtgaccgagaaaga

SEQ ID NO: 94 - DNA, 21 bp, Artificial Sequence
  Description of Artificial Sequence Synthetic primer
gagaaaccagttaattcagcg

SEQ ID NO: 95 - DNA, 20 bp, Artificial Sequence
  Description of Artificial Sequence Synthetic primer
gaggtgtcgtccaagctgga

SEQ ID NO: 96 - DNA, 20 bp, Artificial Sequence
  Description of Artificial Sequence Synthetic primer
gaggtgtcgtccaagctgga

SEQ ID NO: 97 - DNA, 4090 bp, Homo sapiens
gggcgctcccggagtatcagcaaaagggttcgccccgcccacagtgcccggctccccccgggtatcaaaagaaggatcggctccgcccccgggctccccgggggagttgatagaagggtccttcccaccctttgccgtccccactcctgtgcctacgacccaggagcgtgtcagccaaagcatggagaatcaagagaaggcgagtatcgcgggccacatgttcgacgtagtcgtgatcggaggtggcatttcaggactatctgctgccaaactcttgactgaatatggcgttagtgttttggttttagaagctcgggacagggttggaggaagaacatatactataaggaatgagcatgttgattacgtagatgttggtggagcttatgtgggaccaacccaaaacagaatcttacgcttgtctaaggagctgggcatagagacttacaaagtgaatgtcagtgagcgtctcgttcaatatgtcaaggggaaaacatatccatttcggggcgcctttccaccagtatggaatcccattgcatatttggattacaataatctgtggaggacaatagataacatggggaaggagattccaactgatgcaccctgggaggctcaacatgctgacaaatgggacaaaatgaccatgaaagagctcattgacaaaatctgctggacaaagactgctaggcggtttgcttatctttttgtgaatatcaatgtgacctctgagcctcacgaagtgtctgccctgtggttcttgtggtatgtgaagcagtgcgggggcaccactcggatattctctgtcaccaatggtggccaggaacggaagtttgtaggtggatctggtcaagtgagcgaacggataatggacctcctcggagaccaagtgaagctgaaccatcctgtcactcacgttgaccagtcaagtgacaacatcatcatagagacgctgaaccatgaacattatgagtgcaaatacgtaattaatgcgatccctccgaccttgactgccaagattcacttcagaccagagcttccagcagagagaaaccagttaattcagcggcttccaatgggagctgtcattaagtgcatgatgtattacaaggaggccttctggaagaagaaggattactgtggctgcatgatcattgaagatgaagatgctccaatttcaataaccttggatgacaccaagccagatgggtcactgcctgccatcatgggcttcattcttgcccggaaagctgatcgacttgctaagctacataaggaaataaggaagaagaaaatctgtgagctctatgccaaagtgctgggatcccaagaagctttacatccagtgcattatgaagagaagaactggtgtgaggagcagtactctgggggctgctacacggcctacttccctcctgggatcatgactcaatatggaagggtgattcgtcaacccgtgggcaggattttctttgcgggcacagagactgccacaaagtggagcggctacatggaaggggcagttgaggctggagaacgagcagctagggaggtcttaaatggtctcgggaaggtgaccgagaaagatatctgggtacaagaacctgaatcaaaggacgttccagcggtagaaatcacccacaccttctgggaaaggaacctgccctctgtttctggcctgctgaagatcattggattttccacatcagtaactgccctggggtttgtgctgtacaaatacaagctcctgccacggtcttgaagttctgttcttatgctctctgctcactggttttcaataccaccaagaggaaaatattgacaagtttaaaggctgtgtcattgggccatgtttaagtgtactggatttaactacctttggcttaattccaatcattgttaaagtaaaaacaattcaaagaatcacctaattaatttcagtaagatcaagctccatcttatttgtcagtgtagatcaactcatgttaattgatagaataaagccttgtgatcactttctgaaattcacaaagttaaacgtgatgtgctcatcagaaacaatttctgtgtcctgtttttattcccttcaatgcaaaatacatgatgatttcagaaacaaagcatttgactttctgtctgtggaggtggagtaggtgaaggcccagcctgtaactgtcctttttcttcccttaggcaatggtgaactgtcattacagagcctagaggctcacagcctcctggaggaagcagcctccactttggatcaggaaatagtaaaggaaagcagtgttgggggtagcggcatgcagaccctcagaccagaatggggacatcttgtggtctgctgcctcaggaatctcctgaccacttgtagtccctccgacttctctagacatctagtctcagtgctagcttatttgtatttttcctctttcacttcttatggaggagagtgtttaactgagttagaatgttgaaactgacttgctgtgacttatgtgcagctttccagttgagcagaggaaaatagtggcaggactgtcccccaggaggactccctgcttagctctgtgggagaccaactacgactggcatcttctcttccccctggaaggcagctagacaccaatggatccttgtcagttgtaacattctatttcaacttcaggaaagcagcagttttcttttaatttttcctatgaccataaaattagacatacctctcaacttacatatgtcttcaacatggttacctctgcataaatattagcaaagcatgccaatttctcttaagtactgaaatacatatgataaatttgactgttatttgttgagactatcaaacagaaaagaaattagggctctaatttccttaaagcaagctcacttgctttagttgttaagttttataaaagacatgaaattgagtcattttatatatgaaaactaagttctctatcttaggagtaatgtcggcccacaagggtgcccacctcttgttttccccttttaaaaactcagatttttaaaagccctttccaaaggtttcaactgtaaaatacttctttttacaatgtatcaacatatttttatttaaggggaattaacaattgccagggaaaccagccaacccaagtttattatatcattaaccttatcataaattcaaacctaagttgctggaccctggtgtgaggacataaatcttccaaagttttgcctatcctaagagctgcatttttctactgctctttaccttgcattttagctaatttaggagttttgagaatgtattggatacgctccagtacataaggagttgccgcatattatatcagactgctttgagaaatctcatccctagtctattgcagttgtttctattagcttactgattaactcagtcctgacacaccttttgggaaatgctgatttaaacttcttaactggcaacagttggaacagtaatcagtttgctaacatatttaaagtcttgaatgttgaagaactcatgtgatttacccttttcaactttttggaaaacgatttaatttattctaattagattaaccctattaatctatggattgggtatcaaaatgaatgccagtccagatgtgcctagacacgaaattggagctgaggactctcacgatatgcaagttcatccaacgtgaagataccataagctttttctctgaaccagagaaatgaaagtcagtttaagaggctgatagatcttggccctgttaaggcatccacttcacagttctgaaggctgagtcagccccactccacagttaggccaagaattagattttaaaacttcatctgtctgtcccagttaactgttaaataaggcctcatcctccactgaagagtatggattgaaggattgtgaactatgtttagtgtgattgtgaacttggtgcctaatgttccatgtctgaagtttgccccagtgctacacgttggagtatacctatgtgtgtgctttgccactgaagtaagattttgcctgtatggtactgttttgtttgttaataaagtgcactgccacccccaatgcaaaaaaaaaaaaaaaaaa

SEQ ID NO: 98 - DNA, 1370 bp, Homo sapiens
cccaggctgctccagaaacatgagcacaaacgcctcagcctccttccccggcggcaccggcaccggcaccagtacccgcaccagtaccggcaccggcaccagtacccgcaccagtaccggcaccggcaccagtacccgcaccagtaccggcaccggcaccagtacccgcaccagtaccggcaccggcaccgagcgcaaggcggagggcccgcccgaagccgggggcacaactgcccaggtcccgaacccggactccagcttggacgacacctcctacagcctgtccgaatggagcgtccgttctgagtggcggtccgtctcggatccgctagccagttcccagtggagcacgtcctcaactgccgaggccgcctcctggagctccagcatacactccccaatcagcactaccggtcttagcgagagtactgactccgactccaagagtggcctccggggtttcagcgcttacaacccgagcagtcggatccccaagtctaccaccagctcgaactcctccgatggggccgtcacagcctccaatcaggacaccggcattccctgggtattagtaacaggacctaccccgcccgtaaactcccccgtagagtcattgcaagggtctgccttctcctcagggttcagcaccccacggggtttggtaaaaggaccgaccctgcccccggattccaacctgacctcagtgtccgactacacttggatatttgtacggggacctcctatacccaatgacctttcgcaagtgtcaatacaagcacctcctacacccagtaacacccccgagtgtcagtacaagggtctgccgcatcctcagtgtccagcttcccctggggtttggtaccaggaccacctctacccaataacatttccccagtgtcgccacaagcacctcctgcaccccataacatccccccagtgtcaaggcaggcgtctacccccacctcagtgcctgacactccgcggggttcaatacaagaacctcctgcacccagtaatcctttccagctgccgacacaaggacattctaaacctaataactctcgccgagtgtcagtacaagggtccgccccgctctcagtgcccagctccccccgggtatcagctgaaacatcagctccgcccctgggcgctcccggagtatcagcaaaagggttcgccccgcccacagtgcccggctccccccgggtatcaaaagaaggatcggctccgcccccgggctccccgggggagttgatagaagggtccttcccaccctttgccgtccccactcctgtgcctacgacccaggagcgtgtcagccaaagcatggagaatcaagagaaggcgagtatcgcgggccacatgttcga

SEQ ID NO: 99 - DNA, 2350 bp, Homo sapiens
cattgctcttcagcaccagggttctggacagcgccccaagcaggcagctgatcgcacgccccttcctctcaatctccgccagcgctgctactgcccctctagtaccccctgctgcagagaaagaatattacaccgggatccatgcagccagcaatgatgatgttttccagtaaatactgggcacggagagggttttccctggattcagcagtgcccgaagagcatcagctacttggcagctcaacactaaataaacctaactctggcaaaaatgacgacaaaggcaacaagggaagcagcaaacgtgaagctgctaccgaaagtggcaagacagcagttgttttctccttgaagaatgaagttggtggattggtaaaagcactgaggctctttcaggaaaaacgtgtcaacatggttcatattgaatccaggaaatctcggcgaagaagttctgaggttgaaatctttgtggactgtgagtgtgggaaaacagaattcaatgagctcattcagttgctgaaatttcaaaccactattgtgacgctgaatcctccagagaacatttggacagaggaagaagagctagaggatgtgccctggttccctcggaagatctctgagttagacaaatgctctcacagagttctcatgtatggttctgagcttgatgctgaccacccaggatttaaggacaatgtctatcgacagagaagaaagtattttgtggatgtggccatgggttataaatatggtcagcccattcccagggtggagtatactgaagaagaaactaaaacttggggtgttgtattccgggagctctccaaactctatcccactcatgcttgccgagagtatttgaaaaacttccctctgctgactaaatactgtggctacagagaggacaatgtgcctcaactcgaagatgtctccatgtttctgaaagaaaggtctggcttcacggtgaggccggtggctggatacctgagcccacgagactttctggcaggactggcctacagagtgttccactgtacccagtacatccggcatggctcagatcccctctacaccccagaaccagacacatgccatgaactcttgggacatgttccactacttgcggatcctaagtttgctcagttttcacaagaaataggtctggcgtctctgggagcatcagatgaagatgttcagaaactagccacgtgctatttcttcacaatcgagtttggcctttgcaagcaagaagggcaactgcgggcatatggagcaggactcctttcctccattggagaattaaagcacgccctttctgacaaggcatgtgtgaaagcctttgacccaaagacaacttgcttacaggaatgccttatcaccaccttccaggaagcctactttgtttcagaaagttttgaagaagccaaagaaaagatgagggactttgcaaagtcaattacccgtcccttctcagtatacttcaatccctacacacagagtattgaaattctgaaagacaccagaagtattgaaaatgtggtgcaggaccttcgcagcgacttgaatacagtgtgtgatgctttaaacaaaatgaaccaatatctggggatttgatgcctggaactatgttgttgccagcatgatctttttggggcttagcagcagttcagtcaatgtcatataacgcaaataaccttctgtgtcatggcttggctaataagcatgcaattccatatatctataccatcttgtaactcactgtgttagtatataaagcaccataagaaatccaatggcagataacctgaaataacgtattatgtttaaacatcttaaaaagatttgacattcctgcttagtgtccttaaccaaactgcatctagttaaaatttgtaacaaatagccctcttatgagtctcatttatgcccttttctttttcagatctaagcctttcctctgtgttcattagataaaatgaaaaaaagcagtgaagctgtttccattttcaatagtatcagtgttttcacgcattatttgagataaacccagaattgtaggaaacttcccatcacaataacaaaggttcaatattctatttcaaaaattgttgaggtaacacagcagttggaatgatttttaggttgagtatttacacaatgcaagaaaacacctttttacaaatggaattatgtaggttgcgttgaccttgtagaacctgagttatgacaagcttcctgaagtattttggaagatagtacttccggaaaggacattaggaaagactaaacagtggacaatcaatcttgggactatgaattttatgctggaataaagtaaattatcatgttc

SEQ ID NO: 100 - DNA, 11 bp, Artificial Sequence
  Description of Artificial Sequence Synthetic oligonucleotide
attggctgttt

SEQ ID NO: 101 - DNA, 12 bp, Artificial Sequence
  Description of Artificial Sequence Synthetic oligonucleotide
cgcaataagtgc

SEQ ID NO: 102 - DNA, 12 bp, Artificial Sequence
  Description of Artificial Sequence Synthetic oligonucleotide
attggtaagctc

SEQ ID NO: 103 - DNA, 12 bp, Artificial Sequence
  Description of Artificial Sequence Synthetic oligonucleotide
attggtaagttc

SEQ ID NO: 104 - DNA, 12 bp, Artificial Sequence
  Description of Artificial Sequence Synthetic oligonucleotide
attgattagttc

SEQ ID NO: 105 - DNA, 12 bp, Artificial Sequence
  Description of Artificial Sequence Synthetic oligonucleotide
attggttgtttt

SEQ ID NO: 106 - DNA, 12 bp, Artificial Sequence
  Description of Artificial Sequence Synthetic oligonucleotide
attggctggttt

SEQ ID NO: 107 - DNA, 12 bp, Artificial Sequence
  Description of Artificial Sequence Synthetic oligonucleotide
attggtaagctt

SEQ ID NO: 108 - DNA, 12 bp, Artificial Sequence
  Description of Artificial Sequence Synthetic oligonucleotide
cgcaataagtgt

SEQ ID NO: 109 - DNA, 12 bp, Artificial Sequence
  Description of Artificial Sequence Synthetic oligonucleotide
cttgataagttc

SEQ ID NO: 110 - DNA, 12 bp, Artificial Sequence
  Description of Artificial Sequence Synthetic oligonucleotide
attggctgtctc

SEQ ID NO: 111 - DNA, 12 bp, Artificial Sequence
  Description of Artificial Sequence Synthetic oligonucleotide
agcagttgtctc

SEQ ID NO: 112 - DNA, 12 bp, Artificial Sequence
  Description of Artificial Sequence Synthetic oligonucleotide
cttgataagctc

SEQ ID NO: 113 - DNA, 22 bp, Artificial Sequence
  Description of Artificial Sequence Synthetic oligonucleotide
tgtcttgataagttcagtttcg

SEQ ID NO: 114 - DNA, 22 bp, Artificial Sequence
  Description of Artificial Sequence Synthetic oligonucleotide
gagattggctgttttagtcaca

SEQ ID NO: 115 - DNA, 22 bp, Artificial Sequence
  Description of Artificial Sequence Synthetic oligonucleotide
gagattggctgttttagtcaca

SEQ ID NO: 116 - DNA, 22 bp, Artificial Sequence
  Description of Artificial Sequence Synthetic oligonucleotide
gagattggctgttttagtcacg

SEQ ID NO: 117 - DNA, 22 bp, Artificial Sequence
  Description of Artificial Sequence Synthetic oligonucleotide
gagattggctgttttagtcatg

SEQ ID NO: 118 - DNA, 22 bp, Artificial Sequence
  Description of Artificial Sequence Synthetic oligonucleotide
gagattggctgttttagtcacg

SEQ ID NO: 119 - DNA, 22 bp, Artificial Sequence
  Description of Artificial Sequence Synthetic oligonucleotide
gagattggctgttttagtcaca

SEQ ID NO: 120 - DNA, 22 bp, Artificial Sequence
  Description of Artificial Sequence Synthetic oligonucleotide
gagattggctgttttagtcaca

SEQ ID NO: 121 - DNA, 22 bp, Artificial Sequence
  Description of Artificial Sequence Synthetic oligonucleotide
gagattggctgttttagtcacg

SEQ ID NO: 122 - DNA, 22 bp, Artificial Sequence
  Description of Artificial Sequence Synthetic oligonucleotide
gggcgcaataagtgcgagttcg

SEQ ID NO: 123 - DNA, 22 bp, Artificial Sequence
  Description of Artificial Sequence Synthetic oligonucleotide
gagattggctgttttagtcaca

SEQ ID NO: 124 - DNA, 22 bp, Artificial Sequence
  Description of Artificial Sequence Synthetic oligonucleotide
gagattggctgttttaggtttg

SEQ ID NO: 125 - DNA, 22 bp, Artificial Sequence
  Description of Artificial Sequence Synthetic oligonucleotide
gagattggctgttttagtcaca

SEQ ID NO: 126 - DNA, 22 bp, Artificial Sequence
  Description of Artificial Sequence Synthetic oligonucleotide
gagattggctgttttagtcaca

SEQ ID NO: 127 - DNA, 22 bp, Artificial Sequence
  Description of Artificial Sequence Synthetic oligonucleotide
gggattggctgttttagtcaca

SEQ ID NO: 128 - DNA, 22 bp, Artificial Sequence
  Description of Artificial Sequence Synthetic oligonucleotide
gagattggctgttttagtcaca

SEQ ID NO: 129 - DNA, 22 bp, Artificial Sequence
  Description of Artificial Sequence Synthetic oligonucleotide
gagattggctgttttagtcatg

SEQ ID NO: 130 - DNA, 22 bp, Artificial Sequence
  Description of Artificial Sequence Synthetic oligonucleotide
tgtattggtaagttcaatcaca

SEQ ID NO: 131 - DNA, 22 bp, Artificial Sequence
  Description of Artificial Sequence Synthetic oligonucleotide
gagattggctgttttagtcaca

SEQ ID NO: 132 - DNA, 22 bp, Artificial Sequence
  Description of Artificial Sequence Synthetic oligonucleotide
gggagcggtaagctcgattttg

SEQ ID NO: 133 - DNA, 22 bp, Artificial Sequence
  Description of Artificial Sequence Synthetic oligonucleotide
gagattggctgttttagtcaca

SEQ ID NO: 134 - DNA, 22 bp, Artificial Sequence
  Description of Artificial Sequence Synthetic oligonucleotide
gagattggctgttttagtcaca

SEQ ID NO: 135 - DNA, 22 bp, Artificial Sequence
  Description of Artificial Sequence Synthetic oligonucleotide
gagattggctgttttagtcaca

SEQ ID NO: 136 - DNA, 22 bp, Artificial Sequence
  Description of Artificial Sequence Synthetic oligonucleotide
gagattggctgttttagtcaca

SEQ ID NO: 137 - DNA, 22 bp, Artificial Sequence
  Description of Artificial Sequence Synthetic oligonucleotide
gagattggctggtttagtcaca

SEQ ID NO: 138 - DNA, 22 bp, Artificial Sequence
  Description of Artificial Sequence Synthetic oligonucleotide
gggattggctgttttagtttca

SEQ ID NO: 139 - DNA, 22 bp, Artificial Sequence
  Description of Artificial Sequence Synthetic oligonucleotide
gagattggctgttttagtcaca

SEQ ID NO: 140 - DNA, 22 bp, Artificial Sequence
  Description of Artificial Sequence Synthetic oligonucleotide
tgtagcagttgtctcaatttca

SEQ ID NO: 141 - DNA, 22 bp, Artificial Sequence
  Description of Artificial Sequence Synthetic oligonucleotide
tgtattggttgtctcaattttg

SEQ ID NO: 142 - DNA, 22 bp, Artificial Sequence
  Description of Artificial Sequence Synthetic oligonucleotide
gagattggctgttttagtcaca

SEQ ID NO: 143 - DNA, 22 bp, Artificial Sequence
  Description of Artificial Sequence Synthetic oligonucleotide
gagattggctgttttagtcaca

SEQ ID NO: 144 - DNA, 22 bp, Artificial Sequence
  Description of Artificial Sequence Synthetic oligonucleotide
gagattggttgttttagttaca

SEQ ID NO: 145 - DNA, 22 bp, Artificial Sequence
  Description of Artificial Sequence Synthetic oligonucleotide
gagattggctgttttagtcaca

SEQ ID NO: 146 - DNA, 22 bp, Artificial Sequence
  Description of Artificial Sequence Synthetic oligonucleotide
tgtattgattagttcagtywya

SEQ ID NO: 147 - DNA, 22 bp, Artificial Sequence
  Description of Artificial Sequence Synthetic oligonucleotide
tgtattggctgttttagttttg

SEQ ID NO: 148 - DNA, 22 bp, Artificial Sequence
  Description of Artificial Sequence Synthetic oligonucleotide
gagattggctgttttagtcaca

SEQ ID NO: 149 - DNA, 22 bp, Artificial Sequence
  Description of Artificial Sequence Synthetic oligonucleotide
gagattggctgttttagtcaca

SEQ ID NO: 150 - DNA, 22 bp, Artificial Sequence
  Description of Artificial Sequence Synthetic oligonucleotide
gagattggctgttttagtcaca

SEQ ID NO: 151 - DNA, 22 bp, Artificial Sequence
  Description of Artificial Sequence Synthetic oligonucleotide
tggagcggttgtctcaagcatg

SEQ ID NO: 152 - DNA, 22 bp, Artificial Sequence
  Description of Artificial Sequence Synthetic oligonucleotide
tggagcggttgtttcaagcatg

SEQ ID NO: 153 - DNA, 22 bp, Artificial Sequence
  Description of Artificial Sequence Synthetic oligonucleotide
gagattggctgttttaggtttg

SEQ ID NO: 154 - DNA, 22 bp, Artificial Sequence
  Description of Artificial Sequence Synthetic oligonucleotide
gagattggctgttttagtcaca

SEQ ID NO: 155 - DNA, 22 bp, Artificial Sequence
  Description of Artificial Sequence Synthetic oligonucleotide
gagattggctgttttagtcaca

SEQ ID NO: 156 - DNA, 22 bp, Artificial Sequence
  Description of Artificial Sequence Synthetic oligonucleotide
gagattggctgtctcagtcaca

SEQ ID NO: 157 - DNA, 22 bp, Artificial Sequence
  Description of Artificial Sequence Synthetic oligonucleotide
gagattggtaagctcaatcaca

SEQ ID NO: 158 - DNA, 22 bp, Artificial Sequence
  Description of Artificial Sequence Synthetic oligonucleotide
gggcgcaataagtgcgatcaca

SEQ ID NO: 159 - DNA, 22 bp, Artificial Sequence
  Description of Artificial Sequence Synthetic oligonucleotide
tgtattggtaagttcaatcaca

SEQ ID NO: 160 - DNA, 22 bp, Artificial Sequence
  Description of Artificial Sequence Synthetic oligonucleotide
gggattggtaagctcaatcaca

SEQ ID NO: 161 - DNA, 22 bp, Artificial Sequence
  Description of Artificial Sequence Synthetic oligonucleotide
gagattggctgttttagtcaca

SEQ ID NO: 162 - DNA, 22 bp, Artificial Sequence
  Description of Artificial Sequence Synthetic oligonucleotide
gagattggctgttttagtcaca

SEQ ID NO: 163 - DNA, 22 bp, Artificial Sequence
  Description of Artificial Sequence Synthetic oligonucleotide
gagcgcaataggtgcgagtttg

SEQ ID NO: 164 - DNA, 22 bp, Artificial Sequence
  Description of Artificial Sequence Synthetic oligonucleotide
gggcgcaataagtgcgagtttg

SEQ ID NO: 165 - DNA, 22 bp, Artificial Sequence
  Description of Artificial Sequence Synthetic oligonucleotide
tgtattggtaagctcaagtttg

SEQ ID NO: 166 - DNA, 22 bp, Artificial Sequence
  Description of Artificial Sequence Synthetic oligonucleotide
gggcgcaataagtgcgagtttg

SEQ ID NO: 167 - DNA, 22 bp, Artificial Sequence
  Description of Artificial Sequence Synthetic oligonucleotide
gggattggtaagctcaattttg

SEQ ID NO: 168 - DNA, 22 bp, Artificial Sequence
  Description of Artificial Sequence Synthetic oligonucleotide
tgtattggtaagctcaagtttg

SEQ ID NO: 169 - DNA, 22 bp, Artificial Sequence
  Description of Artificial Sequence Synthetic oligonucleotide
gagattggctgttttagtcatg

SEQ ID NO: 170 - DNA, 22 bp, Artificial Sequence
  Description of Artificial Sequence Synthetic oligonucleotide
tgtattggtaagcttagtcaca

SEQ ID NO: 171 - DNA, 22 bp, Artificial Sequence
  Description of Artificial Sequence Synthetic oligonucleotide
gagattggctgttttagtcaca

SEQ ID NO: 172 - DNA, 22 bp, Artificial Sequence
  Description of Artificial Sequence Synthetic oligonucleotide
gagattggctgttttaggtttg

SEQ ID NO: 173 - DNA, 22 bp, Artificial Sequence
  Description of Artificial Sequence Synthetic oligonucleotide
tgtattggtaagctcaagtttg

SEQ ID NO: 174 - DNA, 22 bp, Artificial Sequence
  Description of Artificial Sequence Synthetic oligonucleotide
gagattggtaagctcaagtttg

SEQ ID NO: 175 - DNA, 22 bp, Artificial Sequence
  Description of Artificial Sequence Synthetic oligonucleotide
gagattggtaagctcaatcaca

SEQ ID NO: 176 - DNA, 22 bp, Artificial Sequence
  Description of Artificial Sequence Synthetic oligonucleotide
tgtattggtaagctcaagtttg

SEQ ID NO: 177 - DNA, 22 bp, Artificial Sequence
  Description of Artificial Sequence Synthetic oligonucleotide
tgtattggtaagctcaagtttg

SEQ ID NO: 178 - DNA, 22 bp, Artificial Sequence
  Description of Artificial Sequence Synthetic oligonucleotide
gggattggctgttttagtttca

SEQ ID NO: 179 - DNA, 22 bp, Artificial Sequence
  Description of Artificial Sequence Synthetic oligonucleotide
tgtattggtaagctcaagtttg

SEQ ID NO: 180 - DNA, 22 bp, Artificial Sequence
  Description of Artificial Sequence Synthetic oligonucleotide
tgtattggtaagctcaagtttg

SEQ ID NO: 181 - DNA, 22 bp, Artificial Sequence
  Description of Artificial Sequence Synthetic oligonucleotide
tgtattggtaagttcaatcaca

SEQ ID NO: 182 - DNA, 22 bp, Artificial Sequence
  Description of Artificial Sequence Synthetic oligonucleotide
gggcgcaataagtgcgagtttg

SEQ ID NO: 183 - DNA, 22 bp, Artificial Sequence
  Description of Artificial Sequence Synthetic oligonucleotide
tgtattggtaagttcaatcaca

SEQ ID NO: 184 - DNA, 22 bp, Artificial Sequence
  Description of Artificial Sequence Synthetic oligonucleotide
gagattggctgttttagtcacg

SEQ ID NO: 185 - DNA, 22 bp, Artificial Sequence
  Description of Artificial Sequence Synthetic oligonucleotide
tgtattggtaagctcaatcaca

SEQ ID NO: 186 - DNA, 22 bp, Artificial Sequence
  Description of Artificial Sequence Synthetic oligonucleotide
gagattggctgttttagtcatg

SEQ ID NO: 187 - DNA, 22 bp, Artificial Sequence
  Description of Artificial Sequence Synthetic oligonucleotide
gggattggctgttttagtcaca

SEQ ID NO: 188 - DNA, 22 bp, Artificial Sequence
  Description of Artificial Sequence Synthetic oligonucleotide
tgtattggtaagctcaagtttg

SEQ ID NO: 189 - DNA, 22 bp, Artificial Sequence
  Description of Artificial Sequence Synthetic oligonucleotide
tatattggttgttttagttttg

SEQ ID NO: 190 - DNA, 22 bp, Artificial Sequence
  Description of Artificial Sequence Synthetic oligonucleotide
gggattggctggtttagtcatg

SEQ ID NO: 191 - DNA, 22 bp, Artificial Sequence
  Description of Artificial Sequence Synthetic oligonucleotide
gagattggttgttttgattttg

SEQ ID NO: 192 - DNA, 22 bp, Artificial Sequence
  Description of Artificial Sequence Synthetic oligonucleotide
tgtagcggttgtttcagttttg

SEQ ID NO: 193 - DNA, 22 bp, Artificial Sequence
  Description of Artificial Sequence Synthetic oligonucleotide
gagattggctgttttgatcacg

SEQ ID NO: 194 - DNA, 22 bp, Artificial Sequence
  Description of Artificial Sequence Synthetic oligonucleotide
gagattggctgttttagtcaca

SEQ ID NO: 195 - DNA, 22 bp, Artificial Sequence
  Description of Artificial Sequence Synthetic oligonucleotide
gagattggttgttttagttttg

SEQ ID NO: 196 - DNA, 22 bp, Artificial Sequence
  Description of Artificial Sequence Synthetic oligonucleotide
gggattggctgttttagtttca

SEQ ID NO: 197 - DNA, 22 bp, Artificial Sequence
  Description of Artificial Sequence Synthetic oligonucleotide
tgtattgattagttcagtywyg

SEQ ID NO: 198 - DNA, 22 bp, Artificial Sequence
  Description of Artificial Sequence Synthetic oligonucleotide
tgtcttgataagtttagtcaca

SEQ ID NO: 199 - DNA, 22 bp, Artificial Sequence
  Description of Artificial Sequence Synthetic oligonucleotide
gggattggctgttttagtcaca

SEQ ID NO: 200 - DNA, 22 bp, Artificial Sequence
  Description of Artificial Sequence Synthetic oligonucleotide
gagattggctgttttagtcaca

SEQ ID NO: 201 - DNA, 22 bp, Artificial Sequence
  Description of Artificial Sequence Synthetic oligonucleotide
gggcgcaataagtgcgatcaca

SEQ ID NO: 202 - DNA, 22 bp, Artificial Sequence
  Description of Artificial Sequence Synthetic oligonucleotide
tggagcggttgtttcaattttg

SEQ ID NO: 203 - DNA, 22 bp, Artificial Sequence
  Description of Artificial Sequence Synthetic oligonucleotide
tggagcggttgtctcaattttg

SEQ ID NO: 204 - DNA, 22 bp, Artificial Sequence
  Description of Artificial Sequence Synthetic oligonucleotide
gggcgcaataagtgcgagtttg

SEQ ID NO: 205 - DNA, 22 bp, Artificial Sequence
  Description of Artificial Sequence Synthetic oligonucleotide
gagattggctgttttagtcaca

SEQ ID NO: 206 - DNA, 22 bp, Artificial Sequence
  Description of Artificial Sequence Synthetic oligonucleotide
gggcgcaataagtgcgagtttg

SEQ ID NO: 207 - DNA, 22 bp, Artificial Sequence
  Description of Artificial Sequence Synthetic oligonucleotide
tgtattggtaagttcaatcaca

SEQ ID NO: 208 - DNA, 22 bp, Artificial Sequence
  Description of Artificial Sequence Synthetic oligonucleotide
gggcgcaataagtgcgagtttg

SEQ ID NO: 209 - DNA, 22 bp, Artificial Sequence
  Description of Artificial Sequence Synthetic oligonucleotide
gagattggtaagctcaagtttg

SEQ ID NO: 210 - DNA, 22 bp, Artificial Sequence
  Description of Artificial Sequence Synthetic oligonucleotide
tgtcttgataagctcagtttca

SEQ ID NO: 211 - DNA, 22 bp, Artificial Sequence
  Description of Artificial Sequence Synthetic oligonucleotide
gggcgcaataagtgtagtcaca

SEQ ID NO: 212 - DNA, 22 bp, Artificial Sequence
  Description of Artificial Sequence Synthetic oligonucleotide
gagattggctgttttagtcaca

SEQ ID NO: 213 - DNA, 22 bp, Artificial Sequence
  Description of Artificial Sequence Synthetic oligonucleotide
gggattggctgttttagtcaca

SEQ ID NO: 214 - DNA, 22 bp, Artificial Sequence
  Description of Artificial Sequence Synthetic oligonucleotide
gggcgcaataggtgcgagtttg

SEQ ID NO: 215 - DNA, 22 bp, Artificial Sequence
  Description of Artificial Sequence Synthetic primer
cctctttccagcctcattgttg

SEQ ID NO: 216 - DNA, 36 bp, Artificial Sequence
  Description of Artificial Sequence Synthetic primer
ggccgcgcgccgcgcctctttccagcctcattggtt

SEQ ID NO: 217 - DNA, 22 bp, Artificial Sequence
  Description of Artificial Sequence Synthetic primer
caccctttccaaacctcattga

SEQ ID NO: 218 - DNA, 20 bp, Artificial Sequence
  Description of Artificial Sequence Synthetic primer
caccacatccatccttgcct

SEQ ID NO: 219 - DNA, 23 bp, Artificial Sequence
  Description of Artificial Sequence Synthetic primer
ccctttccaaacctcattgattc

SEQ ID NO: 220 - DNA, 33 bp, Artificial Sequence
  Description of Artificial Sequence Synthetic primer
actgactgcagtcctctttccagcctcattgct

SEQ ID NO: 221 - DNA, 19 bp, Artificial Sequence
  Description of Artificial Sequence Synthetic primer
ctgtcctcagtttgccgga

SEQ ID NO: 222 - DNA, 33 bp, Artificial Sequence
  Description of Artificial Sequence Synthetic primer
gcccggcgcgcgccctgtcctcagtttgcctgg

SEQ ID NO: 223 - DNA, 22 bp, Artificial Sequence
  Description of Artificial Sequence Synthetic primer
cagcacctgtttaagcctcagt

SEQ ID NO: 224 - DNA, 24 bp, Artificial Sequence
  Description of Artificial Sequence Synthetic primer
gcagcaattagttaccaactgtcc

SEQ ID NO: 225 - DNA, 22 bp, Artificial Sequence
  Description of Artificial Sequence Synthetic primer
cagcacctgtttaagcctcagt

SEQ ID NO: 226 - DNA, 24 bp, Artificial Sequence
  Description of Artificial Sequence Synthetic primer
gtgacttctgaatctgacacagaa

SEQ ID NO: 227 - DNA, 20 bp, Artificial Sequence
  Description of Artificial Sequence Synthetic primer
cagcctgcaatcacagcgta

SEQ ID NO: 228 - DNA, 34 bp, Artificial Sequence
  Description of Artificial Sequence Synthetic primer
ggccgcgcggcccgcagcctgcaatcacagcatg

SEQ ID NO: 229 - DNA, 17 bp, Artificial Sequence
  Description of Artificial Sequence Synthetic primer
gaacgcaaccctcgacc

SEQ ID NO: 230 - DNA, 18 bp, Artificial Sequence
  Description of Artificial Sequence Synthetic primer
tcaaaacaagggatggcg

SEQ ID NO: 231 - DNA, 18 bp, Artificial Sequence
  Description of Artificial Sequence Synthetic primer
tgtacctcctcggcgatc

SEQ ID NO: 232 - DNA, 20 bp, Artificial Sequence
  Description of Artificial Sequence Synthetic primer
ccaacccctcctacccgttc

SEQ ID NO: 233 - DNA, 38 bp, Artificial Sequence
  Description of Artificial Sequence Synthetic primer
ggcggcgcgcgcggaaagaggctgatgttaaatatcca

SEQ ID NO: 234 - DNA, 25 bp, Artificial Sequence
  Description of Artificial Sequence Synthetic primer
gaaagaggctgatgttaaatatacg

SEQ ID NO: 235 - DNA, 25 bp, Artificial Sequence
  Description of Artificial Sequence Synthetic primer
gcccaaactacactaagctgatacc

SEQ ID NO: 236 - DNA, 20 bp, Artificial Sequence
  Description of Artificial Sequence Synthetic primer
ccttcctacctccactggca

SEQ ID NO: 237 - DNA, 37 bp, Artificial Sequence
  Description of Artificial Sequence Synthetic primer
ccggccggccggcgccgccttcctacctccactgtcc

SEQ ID NO: 238 - DNA, 21 bp, Artificial Sequence
  Description of Artificial Sequence Synthetic primer
ttgttcaggacagcaatgctg

SEQ ID NO: 239 - DNA, 20 bp, Artificial Sequence
  Description of Artificial Sequence Synthetic primer
gcacaggatgctggagcttc

SEQ ID NO: 240 - DNA, 34 bp, Artificial Sequence
  Description of Artificial Sequence Synthetic primer
ggccggcccgcgcggcacaggatgctggagcgtt

SEQ ID NO: 241 - DNA, 22 bp, Artificial Sequence
  Description of Artificial Sequence Synthetic primer
gatgaagacgagatgaaagcca

SEQ ID NO: 242 - DNA, 20 bp, Artificial Sequence
  Description of Artificial Sequence Synthetic primer
gacccagccctgtcttttgg

SEQ ID NO: 243 - DNA, 35 bp, Artificial Sequence
  Description of Artificial Sequence Synthetic primer
ggccggccggccgcggacccagccctgtctttgga

SEQ ID NO: 244 - DNA, 22 bp, Artificial Sequence
  Description of Artificial Sequence Synthetic primer
ttaacacgttccatgacggaga

SEQ ID NO: 245 - DNA, 19 bp, Artificial Sequence
  Description of Artificial Sequence Synthetic primer
caggtgggaggaggatgca

SEQ ID NO: 246 - DNA, 32 bp, Artificial Sequence
  Description of Artificial Sequence Synthetic primer
ccggccgcgccgccaggtgggaggaggattcg

SEQ ID NO: 247 - DNA, 21 bp, Artificial Sequence
  Description of Artificial Sequence Synthetic primer
ggtatctgtgtgaaggggctg

SEQ ID NO: 248 - DNA, 37 bp, Artificial Sequence
  Description of Artificial Sequence Synthetic primer
ccggccgggcgccgccacagcaaacacaaatctcgct

SEQ ID NO: 249 - DNA, 22 bp, Artificial Sequence
  Description of Artificial Sequence Synthetic primer
cacagcaaacacaaatctcacc

SEQ ID NO: 250 - DNA, 25 bp, Artificial Sequence
  Description of Artificial Sequence Synthetic primer
cgtagaaagtctaaagcaaatggaa

SEQ ID NO: 251 - DNA, 25 bp, Artificial Sequence
  Description of Artificial Sequence Synthetic primer
cgtagaaagtctaaagcaaatggaa

SEQ ID NO: 252 - DNA, 20 bp, Artificial Sequence
  Description of Artificial Sequence Synthetic primer
ccatgggacacagcaaacac

SEQ ID NO: 253 - DNA, 24 bp, Artificial Sequence
  Description of Artificial Sequence Synthetic primer
ggacacagcaaacacaaatctctc

SEQ ID NO: 254 - DNA, 22 bp, Artificial Sequence
  Description of Artificial Sequence Synthetic primer
aaagcaggggacctgtctaaag

SEQ ID NO: 255 - DNA, 38 bp, Artificial Sequence
  Description of Artificial Sequence Synthetic primer
ggccggccggcccggcaaagcaggggacctgtctacaa

SEQ ID NO: 256 - DNA, 21 bp, Artificial Sequence
  Description of Artificial Sequence Synthetic primer
gctttcagaaccaggattgca

SEQ ID NO: 257 - DNA, 21 bp, Artificial Sequence
  Description of Artificial Sequence Synthetic primer
aggtctccatttcctctgtcg

SEQ ID NO: 258 - DNA, 37 bp, Artificial Sequence
  Description of Artificial Sequence Synthetic primer
ccggccgggccgcgccaggtctccatttcctctggca

SEQ ID NO: 259 - DNA, 22 bp, Artificial Sequence
  Description of Artificial Sequence Synthetic primer
cacttttatgttctctggccga

SEQ ID NO: 260 - DNA, 25 bp, Artificial Sequence
  Description of Artificial Sequence Synthetic primer
ggcattgcactttatctcatgtttc

SEQ ID NO: 261 - DNA, 37 bp, Artificial Sequence
  Description of Artificial Sequence Synthetic primer
cgcggccgcgccggcattgcactttatctcatgtgtt

SEQ ID NO: 262 - DNA, 21 bp, Artificial Sequence
  Description of Artificial Sequence Synthetic primer
atgagtgggataagcaagccc

SEQ ID NO: 263 - DNA, 23 bp, Artificial Sequence
  Description of Artificial Sequence Synthetic primer
aagatctccaagcaaagactacc

SEQ ID NO: 264 - DNA, 37 bp, Artificial Sequence
  Description of Artificial Sequence Synthetic primer
cggcggcggcggcgaagatctccaagcaaagactcct

SEQ ID NO: 265 - DNA, 17 bp, Artificial Sequence
  Description of Artificial Sequence Synthetic primer
agagggaggcagggtcc

SEQ ID NO: 266 - DNA, 25 bp, Artificial Sequence
  Description of Artificial Sequence Synthetic primer
ggaaacaggctcatagaaggtatgc

SEQ ID NO: 267 - DNA, 39 bp, Artificial Sequence
  Description of Artificial Sequence Synthetic primer
ccggcgcggccgccggaaacaggctcatagaaggtacga

SEQ ID NO: 268 - DNA, 23 bp, Artificial Sequence
  Description of Artificial Sequence Synthetic primer
ttttgctgagtgaccttaggcaa

SEQ ID NO: 269 - DNA, 23 bp, Artificial Sequence
  Description of Artificial Sequence Synthetic primer
ggcagaagggctcagagatctgg

SEQ ID NO: 270 - DNA, 35 bp, Artificial Sequence
  Description of Artificial Sequence Synthetic primer
ccggccggccggggcagaagggctcagagatccga

SEQ ID NO: 271 - DNA, 18 bp, Artificial Sequence
  Description of Artificial Sequence Synthetic primer
ttccagccttcccccttg

SEQ ID NO: 272 - DNA, 21 bp, Artificial Sequence
  Description of Artificial Sequence Synthetic primer
ttgcaggagtcttcagagtgg

SEQ ID NO: 273 - DNA, 37 bp, Artificial Sequence
  Description of Artificial Sequence Synthetic primer
cgggccggccgcccggttgcaggagtcttcagagcgt

SEQ ID NO: 274 - DNA, 18 bp, Artificial Sequence
  Description of Artificial Sequence Synthetic primer
ctgcaccagaggcagagg

SEQ ID NO: 275 - DNA, 19 bp, Artificial Sequence
  Description of Artificial Sequence Synthetic primer
ccagctgactctccccgac

SEQ ID NO: 276 - DNA, 20 bp, Artificial Sequence
  Description of Artificial Sequence Synthetic primer
gcatgcccattcttctctgg

SEQ ID NO: 277 - DNA, 17 bp, Artificial Sequence
  Description of Artificial Sequence Synthetic primer
ggagtgctgtggagacc

SEQ ID NO: 278 - DNA, 44 bp, Artificial Sequence
  Description of Artificial Sequence Synthetic primer
cgatcacatgtcgtgaactgactgactggtttggcggggctgtc

SEQ ID NO: 279 - DNA, 17 bp, Artificial Sequence
  Description of Artificial Sequence Synthetic primer
agcctgagtcagggccc

SEQ ID NO: 280 - DNA, 15 bp, Artificial Sequence
  Description of Artificial Sequence Synthetic primer
accgcctgctccacg

SEQ ID NO: 281 - DNA, 19 bp, Artificial Sequence
  Description of Artificial Sequence Synthetic primer
cccagaggctgagttttct

SEQ ID NO: 282 - DNA, 20 bp, Artificial Sequence
  Description of Artificial Sequence Synthetic primer
catcctcaaagtgctggacg

SEQ ID NO: 283 - DNA, 34 bp, Artificial Sequence
  Description of Artificial Sequence Synthetic primer
ggcccgggcgccggcatcctcaaagtgctggcca

SEQ ID NO: 284 - DNA, 20 bp, Artificial Sequence
  Description of Artificial Sequence Synthetic primer
agctcactccatcctggacg

SEQ ID NO: 285 - DNA, 19 bp, Artificial Sequence
  Description of Artificial Sequence Synthetic primer
acattgtcctccgcagacg

SEQ ID NO: 286 - DNA, 20 bp, Artificial Sequence
  Description of Artificial Sequence Synthetic primer
gcatgcccattcttctctgg

SEQ ID NO: 287 - DNA, 22 bp, Artificial Sequence
  Description of Artificial Sequence Synthetic primer
gctccactaaagggcaactgta

SEQ ID NO: 288 - DNA, 22 bp, Artificial Sequence
  Description of Artificial Sequence Synthetic primer
tgagggctccactaaaggaggc

SEQ ID NO: 289 - DNA, 20 bp, Artificial Sequence
  Description of Artificial Sequence Synthetic primer
gcatgcccattcttctctgg

SEQ ID NO: 290 - DNA, 17 bp, Artificial Sequence
  Description of Artificial Sequence Synthetic primer
cgctgctaccctgccca

SEQ ID NO: 291 - DNA, 22 bp, Artificial Sequence
  Description of Artificial Sequence Synthetic primer
cagcacctgtttaagcctcagt

SEQ ID NO: 292 - DNA, 35 bp, Artificial Sequence
  Description of Artificial Sequence Synthetic primer
ggggtaccccactggcgagcagacggtgaggaccc

SEQ ID NO: 293 - DNA, 34 bp, Artificial Sequence
  Description of Artificial Sequence Synthetic primer
ggggtacccctgggcagggtagcagcggaacacc

SEQ ID NO: 294 - DNA, 36 bp, Artificial Sequence
  Description of Artificial Sequence Synthetic primer
ggggtaccccttcacagcacctgtttaagcctcagt

SEQ ID NO: 295 - DNA, 35 bp, Artificial Sequence
  Description of Artificial Sequence Synthetic primer
gaagatcttcggggcagagacggcgccggctgctt

SEQ ID NO: 296 - DNA, 32 bp, Artificial Sequence
  Description of Artificial Sequence Synthetic primer
cccaagcttaccagaacgagtgcatcattgcc

SEQ ID NO: 297 - DNA, 32 bp, Artificial Sequence
  Description of Artificial Sequence Synthetic primer
ccgctcgagcgagaacaatggcgagcatctga

SEQ ID NO: 298 - DNA, 19 bp, Artificial Sequence
  Description of Artificial Sequence Synthetic primer
catttaggtgacactatag

SEQ ID NO: 299 - DNA, 40 bp, Homo sapiens
gccagattctgtgtcagattcagaagtcacacacagaaag

SEQ ID NO: 300 - DNA, 38 bp, Mus sp.
gccagcttctatatgagattcaggagtaacagaggaat

SEQ ID NO: 301 - DNA, 25 bp, Rattus sp.
gccagcttctacatgagattcagaa

SEQ ID NO: 302 - DNA, 14 bp, Unknown Organism
  Description of Unknown Organism Rabbit sequence
tgtcagaggcaggg

SEQ ID NO: 303 - DNA, 40 bp, Canis sp.
gccaagctctgtgtcggattccggggtcccagagaggagt

SEQ ID NO: 304 - DNA, 38 bp, Unknown Organism
  Description of Unknown Organism Armadillo sequence
ctcgggctgggtgtcagagtcagggcgcagaaagaact

SEQ ID NO: 305 - DNA, 2643 bp, Homo sapiens
ggcagccgtccggggccgccactctcctcggccggtccctggctcccggaggcggccgcgcgtggatgcggcgggagctggaagcctcaagcagccggcgccgtctctgccccggggcgccctatggcttgaagagcctggccacccagtggctccaccgccctgatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaagggcaactgtactcaccccgaggacatgaaactctgcaccgttatcatgaagtctaatgggagtttcccagtgaacaggcggagagtggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacccggacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctgactctgctgcctgcccgcacagcagcctgcttcccacctccctgcccaggccggccagcctcacccttgcgaaccgtgagcaggaaggcctgggtggatcggcctcctcttcaccccggcaggccctgcagtgttcgcttggctccatgctcctcactgcccgcacaccctcactctgccagggcagtgctagtgagctgggcatggtaccagccctggggctgggccccccagctcaggggcagctcatagagtcccccctcccacctccagtccccctatccttggcaccaaagatgcagccgccttccttgaccttcctctggggctctagggttgctggagcctgagtcagggcccagaggctgagttttctctttgtggggcttggcgtggagcaggcggtggggagagatggacagttcacaccctgcaaggcccacaggaggcaagcaagctctcttgccgaggagccaggcaacttcagtcctgggagacccatgtaaataccagactgcaggttggaccccagagattcccaagccaaaaaccttagctccctcccgcaccccgatgtggacctctactttccaggctagtccggacccacctcaccccgttacagctccccaagtggtttccacatgctctgagaagaggagccctcatcttgaagggcccaggagggtctatggggagaggaactccttggcctagcccaccctgctgccttctgacggccctgcaatgtatcccttctcacagcacatgctggccagcctggggcctggcagggaggtcaggccctggaactctatctgggcctgggctaggggacatcagaggttctttgagggactgcctctgccacactctgacgcaaaaccactttccttttctattccttctggcctttcctctctcctgtttcccttcccttccactgcctctgccttagaggagcccacggctaagaggctgctgaaaaccatctggcctggcctggccctgccctgaggaaggaggggaagctgcagcttgggagagcccctggggcctagactctgtaacatcactatccatgcaccaaactaataaaactttgacgagtcaccttccaggacccctgggtaaaaaaaaaaaaaaa

SEQ ID NO: 306 - DNA, 2556 bp, Homo sapiens
ggcagccgtccggggccgccactctcctcggccggtccctggctcccggaggcggccgcgcgtggatgcggcgggagctggaagcctcaagcagccggcgccgtctctgccccggggcgccctatggcttgaagagcctggccacccagtggctccaccgccctgatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacccggacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctgactctgctgcctgcccgcacagcagcctgcttcccacctccctgcccaggccggccagcctcacccttgcgaaccgtgagcaggaaggcctgggtggatcggcctcctcttcaccccggcaggccctgcagtgttcgcttggctccatgctcctcactgcccgcacaccctcactctgccagggcagtgctagtgagctgggcatggtaccagccctggggctgggccccccagctcaggggcagctcatagagtcccccctcccacctccagtccccctatccttggcaccaaagatgcagccgccttccttgaccttcctctggggctctagggttgctggagcctgagtcagggcccagaggctgagttttctctttgtggggcttggcgtggagcaggcggtggggagagatggacagttcacaccctgcaaggcccacaggaggcaagcaagctctcttgccgaggagccaggcaacttcagtcctgggagacccatgtaaataccagactgcaggttggaccccagagattcccaagccaaaaaccttagctccctcccgcaccccgatgtggacctctactttccaggctagtccggacccacctcaccccgttacagctccccaagtggtttccacatgctctgagaagaggagccctcatcttgaagggcccaggagggtctatggggagaggaactccttggcctagcccaccctgctgccttctgacggccctgcaatgtatcccttctcacagcacatgctggccagcctggggcctggcagggaggtcaggccctggaactctatctgggcctgggctaggggacatcagaggttctttgagggactgcctctgccacactctgacgcaaaaccactttccttttctattccttctggcctttcctctctcctgtttcccttcccttccactgcctctgccttagaggagcccacggctaagaggctgctgaaaaccatctggcctggcctggccctgccctgaggaaggaggggaagctgcagcttgggagagcccctggggcctagactctgtaacatcactatccatgcaccaaactaataaaactttgacgagtcaccttccaggacccctgggtaaaaaaaaaaaaaaa